(19) United States
(12) Patent Application Publication
(10) Pub. No.: US 2012/0219584 A1
(43) Pub. Date: Aug. 30, 2012

(54) PROTECTION AGAINST PANDEMIC AND SEASONAL STRAINS OF INFLUENZA

(75) Inventors: Gary J. Nabel, Washington, DC (US); Chih-Jen Wei, Potomac, MD (US); Zhi-Yong Yang, Potomac, MD (US); Jeffrey Boyington, Clarksburg, MD (US)

(73) Assignee: THE UNITED STATES OF AMERICA as represented by THE SECRETARY, DEPARTMENT OF HEALTH, Bethesda, MD (US)

(21) Appl. No.: 13/500,421

(22) PCT Filed: Oct. 5, 2010
(86) PCT No.: PCT/US2010/051512

§ 371 Date: May 9, 2012

Related U.S. Application Data
(60) Provisional application No. 61/248,835, filed on Oct. 5, 2009.

(51) Int. Cl. A61K 39/145; C07K 14/11; A61P 37/04; C12Q 1/70; A61P 31/16; C12N 15/63; C12N 15/44

(52) U.S. Cl. 424/209.1; 435/320.1; 530/396; 536/23.72; 435/5

(57) ABSTRACT

Immunogens and compositions are provided that encode a protein comprising an influenza A subtype H1 hemagglutinin glycan-shielded receptor binding domain A (RBD A) region and at least one influenza A subtype H1 hemagglutinin antigenic site wherein the antigenic site is not within the RBD-A region. Also provided are immunogens and compositions that encode an immunogenic protein comprising at least one epitope of the RBD-A region of a pandemic influenza A subtype H1 hemagglutinin antigen. Also provided are such proteins, nucleic acids that encode such proteins, and antibodies against such proteins. Also provided are methods to use such immunogens and compositions to elicit a neutralizing antibody immune response against influenza A subtype H1 virus.

This application claims the benefit of U.S. Provisional Patent Application No. 61/248,835, filed Oct. 5, 2009, which is hereby expressly incorporated by reference in its entirety.

### FIELD

The invention relates to influenza immunogens and vaccines. More specifically, the invention relates to influenza immunogens and vaccines comprising nucleic acid molecules or proteins that protect an individual from pandemic and/or seasonal strains of influenza.

### BACKGROUND

New H1N1 influenza viruses have emerged episodically over the last century to cause human pandemics, notably in 1918 and recently in 2009. Pandemic viruses typically evolve into seasonal forms that develop resistance to antibody neutralization, and cross-protection between strains separated by more than three years is uncommon.

The pandemic influenza A (H1N1) 2009 has spread widely after its adaptation to humans. Its rapid global dissemination led to its designation as a pandemic strain by the World Health Organization less than two months after the virus was first identified. The prototypic pandemic H1N1 influenza virus emerged in 1918 and gave rise to seasonal strains that began to diminish in the late 1950s; see, for example, Kilbourne, ED, 2006, Emerg. Infect. Dis. 12, 9-14; Taubenberger, J K, et. Al, 2006, Emerg. Infect. Dis. 12, 15-22. A resurgence of H1N1 viruses occurred in 1977, reestablishing the H1N1 seasonal strains presently in circulation. In contrast to these human-adapted viruses, A (H1N1) 2009 represents a recent cross-species transmission of a virus previously predominantly confined to swine.

Influenza outbreaks are driven by the evolution of diverse viral strains that evade human immunity. Immune protection is mediated predominantly by neutralizing antibodies directed to the hemagglutinin (HA) of these viruses, and co-evolution of HA and neuraminidase (NA) generates variant strains that become resistant to neutralization. Yearly influenza vaccine programs have relied on surveillance of circulating viruses and the identification of strains likely to emerge and cause disease; see, for example, http://www.who.int/csr/disease/influenza/mission/en/.

An alternative approach to influenza prevention is the generation of universal influenza vaccines. This strategy is based on the premise that invariant regions of the viral proteins can be identified as targets of the immune response. Several broadly neutralizing antibodies directed against the viral HA have been identified; see, for example, Okuno Y, et al, 1993, J Virol 67, 2552; Ekiert D C, et al, 2009, Science 324, 246; Sui J, et al, 2009, Nat Struct Mol Biol 16, 265; Kashyap A K, et al, 2008, Proc Natl Acad Sci USA 105, 5986; and the structural basis of antibody recognition and neutralization has been recently elucidated; see, for example, Ekiert D C, et al, ibid; Sui, J et al, 2009, ibid. While this knowledge has identified at least one functionally conserved and constrained target of neutralizing antibodies, it has not been possible to elicit broadly neutralizing antibodies by vaccination.

There remains a need for an influenza vaccine that confers protection not only against the influenza strains that have antigens corresponding to the vaccine but also against heterologous strains, such as pandemic strains and/or seasonal strains. There also remains a need for an influenza vaccine that can reduce or eradicate pandemic strains and/or that can slow or prevent the evolution of seasonal strains.

### SUMMARY

The present invention relates to the novel discovery that two distant, pandemic strains of influenza A virus are able to elicit cross-neutralizing antibodies. Based on this discovery, the present invention describes a mechanism for eliciting protection against pandemic influenza as well as seasonal influenza. Specifically, differences in glycosylation patterns between the hemagglutinin protein of pandemic and seasonal influenza A viruses affect the ability of antibodies to bind to the receptor binding domain of the hemagglutinin protein. Such differences can be used to develop more effective vaccines. One embodiment of the invention comprises a pandemic influenza virus, the hemagglutinin protein of which lacks glycosylation sites normally present in the hemagglutinin protein of non-pandemic influenza viruses. Another embodiment of the invention is a DNA vaccine that encodes at least one epitope from a pandemic virus hemagglutinin protein that lacks glycosylation sites present in the hemagglutinin protein of non-pandemic influenza viruses. In another embodiment, a vaccine of the present invention comprises a peptide comprising at least one epitope from a hemagglutinin protein receptor binding domain that lacks glycosylation sites present in the hemagglutinin protein of non-pandemic influenza viruses. According to the present invention such peptides can be monomers or they can be multimers, such as a trimer. As described herein, the present invention also relates to the use of such vaccines to protect a patient at risk for being infected with influenza A virus from being infected by influenza A virus. It is understood by those in the art that such protection can be prophylactic or it can be therapeutic. The present invention also relates to proteins useful for formulating vaccines of the present invention, as well as nucleic acid molecules encoding such proteins. Such proteins comprise at least a portion of a hemagglutinin protein from a pandemic hemagglutinin protein lacking glycosylation sites normally present in the hemagglutinin protein of non-pandemic influenza viruses. The invention also relates to nucleic acid molecules encoding portions of, or the entire, hemagglutinin protein from both pandemic and non-pandemic influenza A viruses. The invention also describes hemagglutinin proteins from pandemic strains, such as influenza A(H1N1)2009, that have been mutated to contain glycosylation sites, such that the mutant proteins can be used as potential vaccines. Another embodiment of the present invention is a neutralizing antibody that binds one or more epitopes in the receptor binding domain of the hemagglutinin protein, wherein such epitopes are shielded from antibody binding by glycan in the hemagglutinin of non-pandemic influenza virus. Another embodiment of the invention is a method to detect the emergence of non-pandemic strains by detecting glycosylation of the receptor binding domain of the hemagglutinin protein.

The disclosure provides an immunogen comprising a nucleic acid construct comprising a nucleic acid molecule encoding a protein comprising an influenza A subtype H1 hemagglutinin glycan-shielded receptor binding domain A (RBD-A) region and at least one influenza A subtype H1 hemagglutinin antigenic site wherein the antigenic site is not within the RBD-A region. The antigenic site elicits the production of neutralizing antibodies against an antigenic site of a pandemic influenza A subtype H1 HA. The glycan-shielded RBD-A region is homologous to the RBD-A region of the pandemic influenza A subtype H1 HA, with the exception that the glycan-shielded RBD-A region comprises at least one N-linked glycosylation site and the pandemic RBD-A region lacks any N-glycosylation sites. The antigenic site can be an HA1 globular head antigenic site or an HA2 antigenic site. Also included is a composition comprising any of such immunogens. The disclosure also provides a method to elicit a neutralizing antibody immune response against an influenza A subtype H1 virus in a subject; the method comprises administering to the subject any of such immunogens or compositions. Such a method can confer protection against influenza. The disclosure also provides a protein comprising at least a portion of a hemagglutinin antigen having an amino acid sequence selected from the group consisting of SEQ ID NO:27, SEQ ID NO:31, SEQ ID NO:35, SEQ ID NO:39, SEQ ID NO:43, and SEQ ID NO:47.

The disclosure provides an immunogen comprising a nucleic acid construct comprising a nucleic acid molecule that encodes an immunogenic protein comprising at least one epitope of the receptor binding domain A (RBD-A) region of a pandemic influenza A subtype H1 hemagglutinin antigen. The encoded RBD-A region is lacking any N-linked glycosylation site that is present in the RBD-A region of a non-pandemic influenza A subtype H1 hemagglutinin antigen. The immunogenic protein elicits a neutralizing antibody immune response against a homologous pandemic influenza A subtype H1 virus strain and against a heterologous pandemic influenza A subtype H1 virus strain. Also included is a composition comprising any of such immunogens. Also included is a method to elicit a neutralizing antibody immune response against an influenza A subtype H1 virus in a subject comprising administering to the subject any of such immunogens or compositions. Such a method can confer protection against influenza. Also included is a method to reduce pandemic influenza A subtype H1 virus in an animal reservoir comprising administering to animals in the reservoir any of such immunogens or compositions. The disclosure also provides a method to elicit a neutralizing antibody immune response against a pandemic influenza A subtype H1 virus; the method comprises administering to a subject an immunogen comprising a nucleic acid molecule encoding a pandemic influenza A subtype H1 hemagglutinin antigen (HA), wherein the HA is heterologous to the virus against which an immune response is being elicited, and wherein the immunogen elicits the immune response.

The disclosure provides an immunogen comprising nucleic acid construct VRC 9328. Also included is a composition comprising such an immunogen. Also provided is a method to elicit a neutralizing antibody immune response against an influenza A subtype H1 virus in a subject; the method comprises administering to the subject any of such immunogens or compositions.

The disclosure provides a method to detect the emergence of a non-pandemic influenza A subtype H1 virus from a pandemic population of influenza A subtype H1 virus; the method comprises (a) isolating a biological sample containing influenza A virus; and (b) testing the hemagglutinin antigen of the virus for the presence of N-linked glycans at positions corresponding to amino acids 136, 142, 144, 172, 177 and 179 of SEQ ID NO:3. The presence of glycan at any of the positions indicates the emergence of a non-pandemic virus.

### BRIEF DESCRIPTION OF THE FIGURES

FIG. 1. Cross-neutralization, HI reactivity, and specificity of antisera to H1N1 (1918 SC) and A (H1N1) 2009 (CA 04/09) in contrast to a seasonal strain, H1N1 (1999 NC). a, Neutralization activity of antisera from mice immunized with the indicated nucleic acid construct or plasmid containing no insert (control) was measured by luciferase assay with 1918 SC (left), A (H1N1) 2009 (CA 04/09) (middle), or 1999 NC (right) HA-pseudotyped lentiviral vectors. b, Hemagglutination inhibition by antisera from mice immunized with control or the indicated nucleic acid construct was performed with 1918 HA-pseudotyped virus, and H1N1 A (H1N1) 2009 (CA 04/09) and 1999 NC viruses. c, Antisera from mice immunized with a nucleic acid construct encoding 1918 SC, A (H1N1) 2009 (CA 04/09) or 1999 NC HA protein were pre-absorbed with HIV (control), 1918 SC, 2009 (CA 04/09), or 1999 NC HA trimers and the neutralization activities of the pre-absorbed antisera were measured with 1918 SC and 1999 New Caledonia HA-pseudotyped lentiviral vectors. Percent reduction in neutralization was recorded at 1:800 serum dilution.

FIG. 2. Glycosylation patterns of H1N1 HAs. a, Ribbon diagrams (side and top views) of HA depicting N-linked glycosylation on the pandemic 1918 SC and A (H1N1) 2009 strains (left panels) and the seasonal 1999 NC H1N1 strain (right panels). The asparagine side chains of glycosylation sites were rendered as blue CPK models. The glycosylation sites 142 and 177 (1918 numbering) on the top of the RBD are circled by a dotted line. b, Same as in a, except that glycosylations were modeled as mature, sialic acid-containing glycosylations using the GlyProt Server (Bohne-Lang, A. & der Lieth, C. W. GlyProt: in silico glycosylation of proteins. Nucleic Acids Res. 33, W214-W219 (2005)) and rendered as blue stick models. c, Top panel is a table summarizing the presence of glycosylation sites on H1N1 strains during various time frames from 1918 to the present. The numbers indicate residues (1918 numbering) predicted to have glycosylations in at least 50% of the sequences for that particular time period. Glycosylations on the top of the RBD are highlighted yellow. The bottom panel illustrates the placement of these glycosylation sites on ribbon diagrams of 1918 HA. The glycosylations are depicted by side chain CPK models. Glycosylations present in 1918 are colored red and all additional glycosylations after 1918 are colored blue. PDB entry 1RUZ (1918 SC) was used for displaying the H1N1 pandemic strain HAs and the seasonal 1999 NC H1N1 HA was displayed using the structure of the A/PR/8/34 HA (PDB entry 1RU7). All structural panels were generated using the molecular graphics program UCSF Chimera (Pettersen, E. F. et al. UCSF Chimera—a visualization system for exploratory research and analysis. J. Comput. Chem. 25, 1605-1612 (2004)).

FIG. 3. Analysis of purified wild-type HA and glycosylation mutant HA proteins by SDS-PAGE and MALDI-MS. (A) Nucleic acid constructs encoding the ectodomain of wild-type (1918 and 2009) and glycosylation mutant (1918 (2G) and 2009 (2G)) HA proteins were prepared using the mammalian expression vector CMV/R 8κB, and were transiently transfected into 293F renal epithelial cells with or without the presence of swainsonine and kifunsensine to generate recombinant proteins. Glycosylation of 1918 SC and 2009 Ca HA proteins was confirmed by the increase in the size of the HA band (band #1, compare 1918 to 1918 (2G), and 2009 to 2009 (2G)). HA proteins made in the presence of swainsonine and kifunsensine were further treated with Endo H. Upon Endo H digestion (band #20), both wild-type and mutant HA proteins collapsed to the same size, indicating the removal of complex carbohydrates and high mannose N-linked glycans. (B) MALDI-MS analysis of 1918 and 1918 (2G) HA proteins.

FIG. 4. Map and sequence for nucleic acid construct CMV/R 8 kb Influenza A/South Carolina/1/18(H1N1). (SEQ ID NO:1)

FIG. 5. Map and sequence for nucleic acid construct CMV/R 8 kb Influenza A/South Carolina/1/18(H1N1)HA mut A short foldon-His (SEQ ID NO:5).

FIG. 6. Map and sequence for nucleic acid construct CMV/R 8 kb Influenza A/Brevig Mission/1/18(H1N1) NA (SEQ ID NO:9).

FIG. 7. Map and sequence for nucleic acid construct CMV/R 8 kb Influenza A/New Caledonia/20/99 foldon-his AY 289929 (SEQ ID NO:13).

FIG. 8. Map and sequence for nucleic acid construct CMV/R Influenza A H1N1 California/4/2009 HA foldon His (SEQ ID NO:17).

FIG. 9. Map and sequence for nucleic acid construct CMV/R Influenza A H1N1 California/4/2009 NA BlueH (SEQ ID NO:21).

FIG. 10. Map and sequence for nucleic acid construct CMV/R Influenza A California 04 09 HA BlueH (+glycol @ 142 and 177 a.a) (SEQ ID NO:25).

FIG. 11. Map and sequence for nucleic acid construct CMV/R 8 kb Influenza A/South Carolina/1/18(H1N1)HA (+142 and 177 a.a.) (SEQ ID NO:29).

FIG. 12. Map and sequence for nucleic acid construct CMV/R Influenza A California 04 09 HA BlueH (+glycol @ 142 a.a.) (SEQ ID NO:33).

FIG. 13. Map and sequence for nucleic acid construct CMV/R Influenza A California 04 09 HA BlueH (+glycol @ 177 a.a.) (SEQ ID NO:37).

FIG. 14. Map and sequence for nucleic acid construct CMV/R 8 kb Influenza A/South Carolina/1/18(H1N1)HA (+glyc @ 142 a.a.) (SEQ ID NO:41).

FIG. 15. Map and sequence for nucleic acid construct CMV/R 8 kb Influenza A/South Carolina/1/18(H1N1)HA (+glycol @ 177 1.1.) (SEQ ID NO:45).

FIG. 16. Map and sequence for nucleic acid construct CMV/R Influenza A California 04 09 HA BlueH (SEQ ID NO:63).

FIG. 17. Map and sequence for nucleic acid construct CMV/R Influenza A/New Caledonia/20/1999(H1N1) NA (SEQ ID NO:119).

FIG. 18. Map and sequence for nucleic acid construct CMV/R 8 kb Influenza A/New Caledonia 20/99 (H1N1)wt.

FIG. 19. Map and sequence for nucleic acid construct CMV/R H1(Haishu/SWL110/2010/ADG21188 (SEQ ID NO:67).

FIG. 20. Map and sequence for nucleic acid construct CMV/R H1 (Netherlands/1493b/2009/ADJ40554) (SEQ ID NO:71).

FIG. 21. Map and sequence for nucleic acid construct CMV/R H1 (Orenburg/IIV-13/2010/ADF42661) ((SEQ ID NO:)75).

FIG. 22. Map and sequence for nucleic acid construct CMV/R H1 (Orenburg/IIV-13/2010/ADI99498) (SEQ ID NO:79).

FIG. 23. Map and sequence for nucleic acid construct CMV/R H1 (Russia/178/2009/ADA79597) (SEQ ID NO:83).

FIG. 24. Map and sequence for nucleic acid construct CMV/R H1 (Russia/180/2009/ADB81459) (SEQ ID NO:87).

FIG. 25. Map and sequence for nucleic acid construct CMV/R H1 (Salekhard/01/2009/ADA83044) ((SEQ ID NO:91).

FIG. 26. Map and sequence for nucleic acid construct CMV/R H1 (Tallinn/INS183/2010/ADG42553) (SEQ ID NO:95).

FIG. 27. Map and sequence for nucleic acid construct CMV/R H1 (Beijing/SE2649/2009/ADD64214) (SEQ ID NO:99).

FIG. 28. Map and sequence for nucleic acid construct CMV/R H1 (California/VRDL6/2010/ADI99550) (SEQ ID NO:103).

FIG. 29. Map and sequence for nucleic acid construct CMV/R(8 kb)-Influenza H1(A/PR8/8/34 HA/h N144Q (SEQ ID NO:107).

FIG. 30. Map and sequence for nucleic acid construct CMV/R 8 kb Influenza A/New Caledonia/20/99 (H1N1)wt N142Q (SEQ ID NO:111).

FIG. 31. Map and sequence for nucleic acid construct CMV/R 8 kb Influenza A/New Caledonia/20/99 (H1N1)wt N177Q (SEQ ID NO:115).

FIG. 32. Neutralization of wild-type and glycosylated mutant pseudotyped lentiviral vectors by mAb C179. (A) Comparable expression of wild-type and double glycosylation mutants of 1918 SC and 2009 CA HA protein in transfected 293 cells. Cells were stained with C179 mAb or isotype control IgG. (B) Neutralization sensitivities of the indicated wild-type and glycosylation mutant pseudotyped viruses were assessed with C179 mAb. The input wild-type and glycosylation mutant pseudotyped viruses were neutralized by C179 to similar degrees

FIG. 33. Addition of two glycosylation sites to 1918 SC or A (H1N1) 2009 confers resistance to neutralization. (A) Inhibition of neutralizating antibodies to 1918 SC and A (H1N1) 2009 measured on 1918 SC and A (H1N1) 2009 pseudotyped lentiviral vectors or 1999 NC on 1999 lentiviral vectors following absorption of sera with cells expressing the indicated HA protein or without absorption (Control). Percent reduction in neutralization was recorded at 1:400 serum dilution. (B) and (C), Comparable activity of 1918 SC and A (H1N1) 2009, and glycosylation mutants (1918 (2G), and 2009 (2G)) for viral entry using pseudotyped lentiviral vectors (left panels in B and C) and relative resistance of the 2G mutant pseudotyped vs. wild type reporters to neutralization by wild type 1918 SC antisera (middle) or A (H1N1) 2009 antisera (right) derived from DNA-vaccinated mice.

FIG. 34. Neutralization activity of 1918 SC and 209 CA antisera against glycosylated mutant viruses. Both 1918 (2G) and 2009 (2G) viruses were relatively resistant to neutralization by antisera to 1918 SC or 2009 CA. Percent reduction in neutralization was recorded at a 1:1,600 serum dilution.

FIG. 35. Addition of glycosylation sites to 1918 SC confers resistance to neutralization. Neutralization of wild-type and glycosylation mutants of 1918 SC viruses by antisera from mice immunized twice with a nucleic acid vector encoding either wild-type HA or glycosylation mutant 1918 SC HA protein. Percent reduction in neutralization was measured at a 1:200 serum dilution. The sera raised to the 1918 2G HA protein were unable to neutralize the 1999 NC virus.

### DETAILED DESCRIPTION

The present disclosure describes the novel finding of cross-neutralization between two distant pandemic strains, 1918 South Carolina and A(H1N1)2009. Both were resistant to seasonal virus antisera. Pandemic neutralizing antibodies were directed to the receptor binding domain (RBD) of the hemagglutinin (HA). In seasonal strains, this region is shielded by two highly conserved glycosylation sites absent in pandemic strains, and RBD glycosylation of pandemic HAs abrogated neutralization. Collectively, these findings suggest that H1N1 viruses lacking RBD glycosylation have caused pandemics, vaccination directed to these RBDs could protect against similar future pandemics, and glycosylated A(H1N1)2009 could serve as a vaccine to limit the evolution of this virus into seasonal influenza.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

### Glycan-Shielded Immunogens

The present disclosure provides a glycan-shielded immunogen and use thereof to elicit a neutralizing antibody immune response against a pandemic and/or seasonal influenza virus. As used herein, a glycan-shielded immunogen is (a) a nucleic acid molecule that encodes a protein that includes an influenza A subtype H1 hemagglutinin glycan-shielded receptor binding domain A (RBD-A) region and at least one influenza A subtype H1 hemagglutinin antigenic site that is not within an RBD-A region, or (b) a protein that includes an influenza A subtype H1 hemagglutinin glycan-shielded receptor binding domain A (RBD-A) region and at least one influenza A subtype H1 hemagglutinin antigenic site that is not within an RBD-A region.

As used herein, an immunogen is a compound that when administered to a subject elicits an immune response. Such an immune response can be a humoral immune response and/or a cellular immune response to an antigenic site present in an immunogen of the disclosure. As used herein, a humoral immune response refers to an immune response mediated by antibody molecules, including secretory (IgA) or IgG molecules, while a cellular immune response is one mediated by T-lymphocytes and/or other white blood cells. These responses can serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

Influenza strains are typically categorized as influenza A, influenza B or influenza C strains. Influenza A strains are further divided into Group 1 and Group 2 strains. These Groups are further divided into subtypes based on their hemagglutinin proteins: Group 1 influenza A subtypes are H1, H2, H5, H7 and H9. Group 2 influenza A subtypes are H3, H4, H6, H8, H10, H11, H12, H13, H14, H15 and H16. Influenza hemagglutinin proteins are glycoproteins that are found on the surface of influenza viruses and are responsible for binding the virus to the cell that is being infected. Hemagglutinin proteins include antigenic sites that elicit an immune response in subjects infected by their respective virus. Hemagglutinin proteins are also the targets of influenza vaccines. The vaccines are designed, for example, to effect a neutralizing antibody response against the hemagglutinin proteins and thereby protect subjects from viral infection.

As used herein, an influenza hemagglutinin antigen, or HA, is a full-length influenza hemagglutinin protein or any epitope thereof. An epitope of a full-length influenza hemagglutinin protein refers to a portion of such protein that can elicit a neutralizing antibody response against the homologous influenza strain, i.e., a strain from which the HA is derived. In some embodiments, such an epitope can also elicit a neutralizing antibody response against a heterologous influenza strain, i.e., a strain having an HA that is not identical to that of the HA of the immunogen.

Hemagglutinin proteins found on an influenza virus surface are trimers of hemagglutinin protein monomers that are enzymatically cleaved to yield amino-terminal HA1 and carboxy-terminal HA2 polypeptides. The globular head consists exclusively by the major portion of the HA1 polypeptide, whereas the stem that anchors the hemagglutinin protein into the viral lipid envelope is comprised of HA2 and part of HA1. The globular head of a hemagglutinin protein includes two domains: the receptor binding domain (RBD), an ˜148-amino acid residue domain that includes the sialic acid-binding site, and the vestigial esterase domain, a smaller ˜75-amino acid residue region just below the RBD. The top part of the RBD adjacent to the 2,6-sialic acid recognition sites includes a large region (amino acids 131-143, 170-182, 205-215 and 257-262, 1918 numbering) (referred to herein as the RBD-A region) of over 6000 Å² per trimer that is 95% conserved between A/South Carolina/1/1918 (1918 SC) and A/California/04/2009 (2009 CA) pandemic strains. The globular head includes several antigenic sites that include immunodominant epitopes. Examples include the Sa, Sb, Ca₁, Ca₂ and Cb antigenic sites (see, for example, Caton A J et al, 1982, Cell 31, 417-427). The RBD-A region includes the Sa antigenic site and part of the Sb antigenic site.

As described in the present disclosure, the inventors surprisingly discovered that the RBD-A regions of seasonal influenza viruses are shielded by highly conserved glycosylation sites absent in pandemic strains, and that RBD glycosylation of pandemic HAs abrogated neutralization by immune sera directed against pandemic HAs. The RBD-A glycosylation sites are N-linked glycosylation sites that correspond to amino acid residues 142, 144, 172, 177, 179, and 136 of hemagglutinin protein (A/South Carolina/1/1918 (H1N1) HA numbering, or 1918 numbering); see, for example, Table 3. As used herein, a glycan-shielded receptor binding domain A (RBD-A) region is an RBD-A region that comprises an N-linked glycosylation site corresponding to amino acid position 142, 144, 172, 177, 179, and 136 of SEQ ID NO:3 (amino acid sequence of A/South Carolina/1/1918 (H1N1) HA). That is, a glycan-shielded RBD-A region has at least one N-linked saccharide attached to an asparagine (Asn) at one or more of amino acid positions (or residues) 142, 144, 172, 177, 179, and 136 (1918 numbering). It is to be appreciated that an N-linked glycosylation site is typically defined as the three-amino acid motif Asn-X-serine (Ser) or proline (Pro) where X is any amino acid except proline. As used herein, the term N-linked glycosylation site refers to the asparagine attachment site, even though the respective RBD-A region has the entire three-amino acid motif. It is also to be appreciated that SEQ ID NO:3 does not include any glycosylation sites in the RBD-A motif because that amino acid sequence represents the hemagglutinin protein of a pandemic strain; this SEQ ID NO is used simply for reference (i.e., 1918 numbering). In addition, the cited amino acid positions represent those in a full-length hemagglutinin protein although a hemagglutinin antigen of the disclosure need not comprise a full-length hemagglutinin protein.

According to the World Health Organization, “an influenza pandemic occurs when a new influenza virus emerges and spreads around the world, and most people do not have immunity. Viruses that have caused past pandemics typically originated from animal influenza viruses” (http://www.who.int/csedisease/swineflu/frequently_asked_questions/pandemic/en/index. html, Oct. 2, 2010). As used herein, a pandemic influenza A subtype H1 virus is an influenza A subtype H1 virus that has the above-stated characteristics and lacks N-linked glycosylation sites, and hence is not glycosylated, in the RBD-A region. Pandemic influenza viruses often represent a cross-species transmission of virus predominantly confined to a non-human animal reservoir. For example, influenza A (H1N1) 2009 represents a recent cross-species transmission of a virus previously predominantly confined to swine. Pandemic influenza viruses typically comprise an immunodominant RBD-A antigenic site that elicits immune responses targeted primarily toward the RBD-A region, and typically are not neutralized by an immune response against previous seasonal influenza vaccines.

One embodiment of the disclosure is an immunogen that comprises a nucleic acid construct comprising a nucleic acid molecule encoding a protein comprising an influenza H1 hemagglutinin glycan-shielded receptor binding domain A (RBD-A) region and at least one influenza A subtype H1 hemagglutinin antigenic site selected from the group consisting of an HA1 globular head antigenic site and an HA2 antigenic site, wherein the antigenic site is not within the RBD-A region. Such an antigenic site elicits the production of neutralizing antibodies against an antigenic site of a pandemic influenza A subtype H1 HA. Such a glycan-shielded RBD-A region is homologous to the RBD-A region of a pandemic influenza A subtype H1 HA, with the exception that the glycan-shielded RBD-A region comprises at least one N-linked glycosylation site and the pandemic RBD-A region lacks any N-glycosylation sites.

As used herein, the phrase “elicits the production of neutralizing antibodies against an antigenic site of a pandemic influenza A subtype H1 HA” means that the antigenic site can effect an immune response that results in neutralizing antibodies (i.e., a neutralizing antibody immune response) against a HA of a pandemic influenza A subtype H1 virus. Due to the nature of the immunogen, such an immune response is elicited against an antigenic site not within the RBD-A region of the pandemic HA. That is, the encoded protein comprises a RBD-A region that, due to glycan-shielding (i.e., masking, hiding) antigenic epitopes on the RBD-A region, does not stimulate a neutralizing antibody immune response against itself; instead, the encoded protein, also having an antigenic site that is not within the RBD-A region, directs the immune response away from the RBD-A region and toward the antigenic site. Such an immune response can have utility not only against the pandemic influenza A subtype H1 virus but also against an influenza virus that is evolving from the pandemic influenza virus into a seasonal influenza virus. Such an immune response can also have utility against a seasonal influenza virus. Without being bound by theory, it is believed that pandemic viral strains evolve to evade immune responses directed against them by acquiring mutations that encode glycosylation sites to shield the highly immunodominant epitopes in the RBD-A region that are neutralized by immune sera raised against pandemic viruses. It is to be appreciated that influenza strains also evolve by acquiring mutations to otherwise change the amino acid sequences of the HAs, thereby evading previously generated immune responses. In one embodiment, the pandemic influenza A subtype H1 virus is the most recent to have caused pandemic infection.

In one embodiment, an antigenic site is an influenza A subtype H1 HA1 globular head antigenic site, wherein the antigenic site is not within the RBD-A region. In one embodiment, an antigenic site is an influenza A subtype H1 HA2 antigenic site. In one embodiment, an antigenic site is an influenza A subtype H1 globular head antigenic site, such as, but not limited to, an Sb antigenic site, an Ca₁ antigenic site, an Ca₂ antigenic site or an Cb antigenic site. It is to be appreciated that amino acid residues 142 and 177 (1918 numbering) of the RBD-A region of an influenza A subtype H1 hemagglutinin protein are within the Sa antigenic site. In one embodiment, the nucleic acid construct encodes more than one antigenic site. Any antigenic site in the protein must have a proper three-dimensional structure to elicit a neutralizing antibody immune response against an influenza A subtype H1 virus. Typically, the protein forms a trimer analogous to what natural hemagglutinin proteins do. Assays to determine that the protein does elicit such a response are known to those skilled in the art.

As used herein, the phrase the “glycan-shielded RBD-A region is homologous to the RBD-A region of said pandemic influenza A subtype H1 HA” means that the amino acid sequence of the glycan-shielded RBD-A region is at least 80% identical to the amino acid sequence of the pandemic RBD-A region. In one embodiment, the amino acid sequence of the glycan-shielded RBD-A region is at least about 85% identical to the amino acid sequence of the pandemic RBD-A region. In one embodiment, the amino acid sequence of the glycan-shielded RBD-A region is at least about 90% identical to the amino acid sequence of the pandemic RBD-A region. In one embodiment, the amino acid sequence of the glycan-shielded RBD-A region is at least about 95% identical to the amino acid sequence of the pandemic RBD-A region.

As used herein a nucleic acid construct is a recombinant expression vector, i.e., a vector linked to a nucleic acid molecule encoding a protein such that the nucleic acid molecule can effect expression of the protein when the nucleic acid construct is administered to, for example, a subject or an organ, tissue or cell. The vector also enables transport of the nucleic acid molecule to a cell within an environment, such as, but not limited to, an organism, tissue, or cell culture. A nucleic acid construct of the present disclosure is produced by human intervention. The nucleic acid construct can be DNA, RNA or variants thereof. The vector can be a DNA plasmid, a viral vector, or other vector. In one embodiment, a vector can be a cytomegalovirus (CMV), retrovirus, adenovirus, adeno-associated virus, herpes virus, vaccinia virus, poliovirus, sindbis virus, or any other DNA or RNA virus vector. In one embodiment, a vector can be a pseudotyped lentiviral or retroviral vector. In one embodiment, a vector can be a DNA plasmid. In one embodiment, a vector can be a DNA plasmid comprising viral components and plasmid components to enable nucleic acid molecule delivery and expression. Methods for the construction of nucleic acid constructs of the present disclosure are well known. See, for example, Molecular Cloning: a Laboratory Manual, 3^{rd} edition, Sambrook et al. 2001 Cold Spring Harbor Laboratory Press, and Current Protocols in Molecular Biology, Ausubel et al. eds., John Wiley & Sons, 1994. In one embodiment, the vector is a DNA plasmid, such as a CMV/R plasmid such as CMV/R or CMV/R 8 κB (also referred to herein as CMV/R 8 kb). Examples of CMV/R and CMV/R 8 kb are provided herein. CMV/R is also described in U.S. Pat. No. 7,094,598 B2, issued Aug. 22, 2006. It is to be appreciated that an immunogen can comprise one nucleic acid construct or more than one nucleic acid construct.

As used herein, a nucleic acid molecule comprises a nucleic acid sequence that encodes a hemagglutinin antigen. A nucleic acid molecule can be produced recombinantly, synthetically, or by a combination of recombinant and synthetic procedures. A nucleic acid molecule of the disclosure can have a wild-type nucleic acid sequence or a codon-modified nucleic acid sequence to, for example, incorporate codons better recognized by the human translation system. In one embodiment, a nucleic acid molecule can be genetically-engineered to introduce codons encoding different amino acids, such as to introduce codons that encode an N-linked glycosylation site. Methods to produce nucleic acid molecules of the disclosure are known in the art, particularly once the nucleic acid sequence is know. A nucleic acid molecule of the present disclosure does not include an entire influenza virus genome. It is to be appreciated that a nucleic acid construct can comprise one nucleic acid molecule or more than one nucleic acid molecule. It is also to be appreciated that a nucleic acid molecule can encode one protein or more than one protein.

One embodiment of the disclosure is a nucleic acid molecule that encodes a protein comprising an influenza A subtype H1 hemagglutinin glycan-shielded receptor binding domain A (RBD-A) region and at least one influenza A subtype H1 hemagglutinin antigenic site, wherein said antigenic site is not within the RBD-A region, wherein the antigenic site elicits the production of neutralizing antibodies against an antigenic site of a pandemic influenza A subtype H1 HA, and wherein the glycan-shielded RBD-A region is homologous to the RBD-A region of the pandemic influenza A subtype H1 HA, with the exception that that glycan-shielded RBD-A region comprises at least one N-linked glycosylation site and the pandemic RBD-A region lacks any N-glycosylation sites. In one embodiment, the nucleic acid molecule encodes an influenza A subtype H1 HA1 region. In one embodiment, the nucleic acid molecule encodes the globular head of an influenza A subtype H1 hemagglutinin protein. In one embodiment, the nucleic acid molecule encodes a full-length influenza A subtype H1 hemagglutinin protein or a mature version thereof.

In one embodiment, the glycan-shielded RBD-A region of the protein comprises an RBD-A region of an influenza A subtype H1 HA that has an N-linked glycosylation site within the RBD-A region. In one embodiment such glycan-shielded RBD-A region elicits an immune response in which neutralizing antibodies are directed against an antigenic site within HA that is not within the RBD-A region. In one embodiment, an immunogen encodes a protein, the RBD-A region of which comprises a glycan-shielded RBD-A region of at least one of the following HAs: SEQ ID NO:27, SEQ ID NO:31, SEQ ID NO:35, SEQ ID NO:39, SEQ ID NO:43, and SEQ ID NO:47. For reference:

| | | Short |
|---|---|---|
| SEQ ID NO | Hemagglutinin antigen (HA) | name |
| SEQ ID NO: | A/California/04/2009 (H1N1) HA/h BlueH | 2009 CA |
| 27 | w/N-linked glycosylation sites at AA 142 | [2G-142 + |
| | and AA 177 | 177] |
| SEQ ID NO: | A/South Carolina/1/1918 (H1N1) HA w/N- | 1918 SC |
| 31 | linked glycosylation sites at AA 142 and | [2G-142 + |
| | AA 177 | 177] |
| SEQ ID NO: | A/California/04/2009 (H1N1) HA/h BlueH | 2009 CA |
| 35 | w/N-linked glycosylation site at AA 142 | [1G-142] |
| SEQ ID NO: | A/California/04/2009 (H1N1) HA/h BlueH | 2009 CA |
| 39 | w/N-linked glycosylation site at AA 177 | [1G-177] |
| SEQ ID NO: | A/South Carolina/1/1918 (H1N1) HA w/N- | 1918 SC |
| 43 | linked glycosylation site at AA 142 | [1G-142] |
| SEQ ID NO: | A/South Carolina/1/1918 (H1N1) HA w/N- | 1918 SC |
| 47 | linked glycosylation site at AA 177 | [1G-177] |

It is to be noted that the phrase “w/N-linked glycosylation site” means that an N-linked glycosylation site has been genetically engineered (at the DNA level) into the respective HA. For example, “A/California/04/2009 (H1N1) HA/h BlueH w/ N-linked glycosylation sites at AA 142 and AA 177” means an influenza A/California/04/2009 (H1N1) HA/h BlueH genetically engineered to include the 3-amino acid N-glycosylation site motif from amino acids 142-144 and 177-179 (1918 numbering). A short hand notation for this HA is 2009 CA [2G-142+177].

In one embodiment, the protein comprises an HA1 polypeptide of an influenza A subtype H1 HA. In one embodiment, the protein comprises an HA1 polypeptide of an HA having an amino acid sequence selected from the group consisting of SEQ ID NO:27, SEQ ID NO:31, SEQ ID NO:35, SEQ ID NO:39, SEQ ID NO:43, and SEQ ID NO:47.

In one embodiment, the protein comprises a globular head of an influenza A subtype H1 HA. In one embodiment, the protein comprises a globular head of an HA having an amino acid sequence selected from the group consisting of SEQ ID NO:27, SEQ ID NO:31, SEQ ID NO:35, SEQ ID NO:39, SEQ ID NO:43, and SEQ ID NO:47.

In one embodiment, the protein comprises an influenza A subtype H1 HA having an amino acid sequence selected from the group consisting of SEQ ID NO:27, SEQ ID NO:31, SEQ ID NO:35, SEQ ID NO:39, SEQ ID NO:43, and SEQ ID NO:47. In one embodiment, the protein comprises amino acid sequence SEQ ID NO:27 or SEQ ID NO:31.

In one embodiment, the protein comprises a glycan-shielded RBD-A region comprising at least one of the following regions: (a) amino acids 131-143 from SEQ ID NO:27 or SEQ ID NO:31; (b) amino acids 170-182 from SEQ ID NO:27 or SEQ ID NO:31; (c) amino acids 205-215 from SEQ ID NO:27 or SEQ ID NO:31; (d) amino acids 257-262 from SEQ ID NO:27 or SEQ ID NO:31; or (e) amino acids 131-146 from SEQ ID NO:27 or SEQ ID NO:31. In one embodiment, the protein comprises a glycan-shielded RBD-A region comprising: (a) amino acids 131-143 from SEQ ID NO:27 or SEQ ID NO:31; (b) amino acids 170-182 from SEQ ID NO:27 or SEQ ID NO:31; (c) amino acids 205-215 from SEQ ID NO:27 or SEQ ID NO:31; and (d) amino acids 257-262 from SEQ ID NO:27 or SEQ ID NO:31. In one embodiment, the protein comprises a glycan-shielded RBD-A region comprising (a) amino acids 131-143 from SEQ ID NO:27; (b) amino acids 170-182 from SEQ ID NO:27; (c) amino acids 205-215 from SEQ ID NO:27; and (d) amino acids 257-262 from SEQ ID NO:27. In one embodiment, the protein comprises a glycan-shielded RBD-A region comprising (a) amino acids 131-143 from SEQ ID NO:31; (b) amino acids 170-182 from SEQ ID NO:31; (c) amino acids 205-215 from SEQ ID NO:31; and (d) amino acids 257-262 from SEQ ID NO:31.

In one embodiment, the nucleic acid construct comprises a DNA plasmid that is operatively linked to a nucleic acid molecule encoding at least one of the proteins disclosed herein, such that the nucleic acid molecule expresses the protein. In one embodiment, the DNA plasmid comprises a CMV plasmid, such as CMV/R or CMV/R 8 kb. In one embodiment, the nucleic acid construct comprises a CMV/R plasmid operatively linked to a nucleic acid molecule encoding such protein. In one embodiment, the nucleic acid construct comprises a CMV/R 8 kb plasmid operatively linked to a nucleic acid molecule encoding such protein.

One embodiment is an immunogen comprising a nucleic acid construct having nucleic acid sequence SEQ ID NO:25 (VRC 9446), SEQ ID NO:29 (VRC 9449), SEQ ID NO:33 (VRC 9444), SEQ ID NO:37 (VRC 9445), SEQ ID NO:41 (VRC 9447), or SEQ ID NO:45 (VRC 9448).

Another embodiment of the disclosure is a glycan-shielded immunogen comprising a protein that comprises an influenza A subtype H1 hemagglutinin glycan-shielded receptor binding domain A (RBD-A) region and at least one influenza A subtype H1 hemagglutinin antigenic site, wherein the antigenic site is not within the RBD-A region, wherein the antigenic site elicits the production of neutralizing antibodies against an antigenic site of a pandemic influenza A subtype H1 HA, and wherein the glycan-shielded RBD-A region is homologous to the RBD-A region of the pandemic influenza A subtype H1 HA, with the exception that that glycan-shielded RBD-A region comprises at least one N-linked glycosylation site and the pandemic RBD-A region lacks any N-glycosylation sites. It is to be appreciated that such a protein can comprise any of the proteins described above as being encoded by the nucleic acid molecules of those embodiments.

The present disclosure also provides antibodies that neutralize influenza A subtype H1 antigenic sites of HA. Such antibodies are produced by administering a glycan-shielded immunogen as disclosed herein to an animal and harvesting immune sera or monoclonal antibodies, using techniques known to those skilled in the art. As such, the antibodies can be polyclonal or monoclonal. Such antibodies have utility against pandemic, evolving and seasonal influenza A subtype H1 viruses.

The present disclosure also provides compositions that comprise a glycan-shielded immunogen as disclosed herein. One embodiment is a composition comprising an immunogen comprising a nucleic acid construct as described above. Another embodiment is a composition comprising a protein as described above. Another embodiment is a composition comprising a glycan-shielded immunogen and another influenza vaccine that protects against influenza virus, such as, but not limited to, a nucleic acid immunogen, a protein immunogen, a subunit immunogen, an inactivated virus immunogen, a subvirion immunogen, or an attenuated virus immunogen. Such a vaccine can be monovalent or multivalent.

Non-limiting examples of such compositions include the following: In one embodiment, the composition comprises (a) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein and (b) a pandemic influenza A hemagglutinin protein or a nucleic acid molecule encoding a pandemic influenza A hemagglutinin protein. In one embodiment, the composition comprises (a) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein and (b) nucleic acid construct VRC 9328 that encodes A/California/04/2009 (H1N1) HA. In one embodiment the composition comprises (a) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein and (b) an immunogen comprising at least one nucleic acid molecule encoding at least one influenza hemagglutinin antigen (HA) selected from the group consisting of influenza A group 1 HA, influenza A group 2 HA, influenza B group HA, and influenza C group HA. In one embodiment, the composition comprises (a) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein and (b) an immunogen comprising at least one hemagglutinin antigen (HA) selected from the group consisting of influenza A group 1 HA, influenza A group 2 HA, influenza B group HA, and influenza C group HA. In one embodiment, the composition comprises (a) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein, (b) a pandemic influenza A hemagglutinin protein or a nucleic acid molecule encoding a pandemic influenza A hemagglutinin protein, and (c) a seasonal influenza vaccine.

As used herein, a seasonal influenza vaccine refers to a vaccine that is developed for a flu season as described herein. Typically, a seasonal influenza vaccine includes a group 1 influenza A strain, a group 2 influenza A strain, and an influenza B strain. Group 1 influenza A strains include those strains having a H1, H2, H5, H7 or H9 HA subtype. Group 2 influenza A strains include those strains having a H3, H4, H6, H8, H10, H11, H12, H13, H14, H15 or H16 HA subtype. For example, the 2006-2007 influenza virus vaccine includes HA from A/New Caledonia/20/1999 (H1N1), A/Wisconsin/67/2005 (H3N2) and B/Malaysia/256/2004; the 2007-2008 influenza virus vaccine includes HA from A/Solomon Islands/3/2006 (H1N1), A/Wisconsin/67/2005 (H3N2) and B/Malaysia/2506/2004); the 2008-2009 seasonal influenza vaccine includes HA from A/Brisbane/59/2007 (H1N1); A/Brisbane/10/2007 (H3N2) and B/Florida/4/2006; and the 2009-2010 seasonal influenza vaccine includes HA from a A/Brisbane/59/2007 (H1N1)-like virus, a A/Brisbane/10/2007 (H3N2)-like virus, and a B/Brisbane/60/2008-like virus.

The present disclosure also provides proteins comprising a glycan-shielded RBD-A region. Such proteins are produced by genetically-engineering one or more N-linked glycosylation sites into an RBD-A region of a hemagglutinin antigen from a pandemic influenza A subtype H1 virus. One embodiment is a protein comprising at least a portion of a hemagglutinin antigen having an amino acid sequence selected from the group consisting of SEQ ID NO:27, SEQ ID NO:31, SEQ ID NO:35, SEQ ID NO:39, SEQ ID NO:43, and SEQ ID NO:47. Such portion can comprise at least one of the following regions: (a) amino acids 131-143 from SEQ ID NO:27 or SEQ ID NO:31; (b) amino acids 170-182 from SEQ ID NO:27 or SEQ ID NO:31; (c) amino acids 205-215 from SEQ ID NO:27 or SEQ ID NO:31; (d) amino acids 257-262 from SEQ ID NO:27 or SEQ ID NO:31; or (e) amino acids 131-146 from SEQ ID NO:27 or SEQ ID NO:31. In one embodiment, such portion can comprise (a) amino acids 131-143 from SEQ ID NO:27; (b) amino acids 170-182 from SEQ ID NO:27; (c) amino acids 205-215 from SEQ ID NO:27; and (d) amino acids 257-262 from SEQ ID NO:27. In one embodiment, such portion can comprise (a) amino acids 131-143 from SEQ ID NO:31; (b) amino acids 170-182 from SEQ ID NO:31; (c) amino acids 205-215 from SEQ ID NO:31; and (d) amino acids 257-262 from SEQ ID NO:31. One embodiment is a protein that comprises a HA1 polypeptide from a hemagglutinin antigen comprising SEQ ID NO:27, SEQ ID NO:31, SEQ ID NO:35, SEQ ID NO:39, SEQ ID NO:43, and SEQ ID NO:47. One embodiment is a protein that comprises a receptor binding domain from a hemagglutinin antigen comprising SEQ ID NO:27, SEQ ID NO:31, SEQ ID NO:35, SEQ ID NO:39, SEQ ID NO:43, and SEQ ID NO:47. One embodiment is a protein that comprises a RBD-A region from a hemagglutinin antigen comprising SEQ ID NO:27, SEQ ID NO:31, SEQ ID NO:35, SEQ ID NO:39, SEQ ID NO:43, and SEQ ID NO:47. In one embodiment, the RBD-A region is from a hemagglutinin antigen comprising SEQ ID NO:27. In one embodiment, the RBD-A region is from a hemagglutinin antigen comprising SEQ ID NO:31. One embodiment is a protein comprising SEQ ID NO:27, SEQ ID NO:31, SEQ ID NO:35, SEQ ID NO:39, SEQ ID NO:43, and SEQ ID NO:47. One embodiment is a protein comprising SEQ ID NO:27. One embodiment is a protein comprising SEQ ID NO:31. The present disclosure also provides a nucleic acid molecule encoding any of these proteins. Now that these proteins have been described, for example by their amino acid sequences, one skilled in the art can produce such proteins and nucleic acid molecules. Such proteins can be produced by recombinant DNA technology or by chemical synthesis. One skilled in the art can also take the RBD-A regions of hemagglutinin antigens from other pandemic influenza A subtype H1 viruses and produce glycan-shielded RBD-A regions therefrom.

The present disclosure provides a method to elicit a neutralizing antibody immune response against an influenza A subtype H1 virus in a subject comprising administering to the subject an immunogen or composition comprising a nucleic acid construct comprising a nucleic acid molecule that encodes a protein comprising an influenza A subtype hemagglutinin glycan-shielded receptor binding domain A (RBD-A) region and at least one influenza A subtype H1 hemagglutinin antigenic site, wherein said antigenic site is not within the RBD-A region, wherein the antigenic site elicits the production of neutralizing antibodies against an antigenic site of a pandemic influenza A subtype H1 HA, and wherein the glycan-shielded RBD-A region is homologous to the RBD-A region of the pandemic influenza A subtype H1 HA, with the exception that that glycan-shielded RBD-A region comprises at least one N-linked glycosylation site and the pandemic RBD-A region lacks any N-glycosylation sites. Examples of suitable immunogens and compositions are disclosed herein. In one embodiment, such immunogens or compositions elicit antibodies that neutralize a pandemic influenza A subtype H1 virus. In one embodiment, such immunogens or compositions elicit antibodies that neutralize an evolving influenza A subtype H1 virus. An evolving influenza virus is a virus that is mutating to evade the immune response generated by a pandemic influenza virus. In one embodiment the evolving virus has acquired an N-linked glycosylation site in the RBD-A region. In one embodiment, such immunogens or compositions elicit antibodies that neutralize a seasonal influenza A subtype H1 virus. One embodiment is a method to protect a subject from influenza A subtype H1 infection comprising administering to the subject any of such immunogens or compositions. Such protection can be either therapeutic (i.e., to treat an influenza virus infection) or prophylactic (i.e., to protect a subject from disease caused by influenza virus or to prevent or reduce infection by influenza virus). Depending on the nature of the immunogens or compositions, protection against other influenza virus types, groups and/or subtypes can also be achieved.

The present disclosure also includes administering a protein comprising an influenza A subtype H1 hemagglutinin glycan-shielded receptor binding domain A (RBD-A) region and at least one influenza A subtype H1 hemagglutinin antigenic site, wherein said antigenic site is not within the RBD-A region, wherein the antigenic site elicits the production of neutralizing antibodies against an antigenic site of a pandemic influenza A subtype H1 HA, and wherein the glycan-shielded RBD-A region is homologous to the RBD-A region of the pandemic influenza A subtype H1 HA, with the exception that that glycan-shielded RBD-A region comprises at least one N-linked glycosylation site and the pandemic RBD-A region lacks any N-glycosylation sites, or a composition comprising such a protein. Such proteins can elicit the production of neutralizing antibodies against pandemic, evolving or seasonal influenza virus as described above. Such proteins can protect a subject from influenza as described above. A composition comprising antibodies that neutralize against such antigenic sites can also be administered. Such antibodies can protect a subject from influenza as described above.

As used herein, a subject refers to any human or other animal susceptible to influenza infection. Examples include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are included. An infected subject is a subject that has been exposed to an influenza virus that causes a natural immune response in the subject. A vaccinated subject is a subject that has been administered an immunogen or vaccine that is intended to provide a protective effect against an influenza virus.

### Cross-Protective Pandemic Immunogens

The present disclosure provides immunogens against pandemic influenza A subtype H1 viruses that can elicit an immune response not only against the homologous pandemic influenza A subtype H1 virus strain but also against heterologous pandemic influenza A subtype H1 virus strains. These immunogens either encode a protein comprising a non-glycosylated receptor binding domain A (RBD-A) region of a pandemic influenza A subtype H1 hemagglutinin antigen or comprise such a protein. Due to their ability to protect against homologous and heterologous pandemic influenza A subtype strains, even those that appeared in the human population more than 90 years apart, such immunogens are referred to herein as cross-protective pandemic immunogens.

One embodiment of the disclosure is an immunogen comprising a nucleic acid construct comprising a nucleic acid molecule that encodes an immunogenic protein comprising at least one epitope of the receptor binding domain A (RBD-A) region of a pandemic influenza A subtype H1 hemagglutinin antigen, wherein the encoded RBD-A region is lacking any N-linked glycosylation site that is present in the RBD-A region of a non-pandemic influenza A subtype H1 hemagglutinin antigen, wherein the immunogenic protein can elicit a neutralizing antibody immune response against a homologous pandemic influenza A subtype H1 virus strain and against a heterologous pandemic influenza A subtype H1 virus strain. The terms immunogen, nucleic acid construct, nucleic acid molecule, RBD-A region, hemagglutinin antigen, pandemic influenza virus, N-linked glycosylation site, neutralizing antibody immune response, homologous strain and heterologous strain have been described elsewhere herein. As used herein, an epitope of a RBD-A region is a three-dimensional amino acid structure that can elicit a neutralizing antibody response against the non-glycosylated RBD-A region of a pandemic influenza A subtype H1 virus. Such epitope can be located entirely within a RBD-A region or can be located partly in a RBD-A region and partly in a nearby region of the globular head of an influenza A subtype H1 hemagglutinin protein.

The pandemic influenza A subtype H1 hemagglutinin antigen can be any pandemic influenza A subtype H1 hemagglutinin antigen, hemagglutinin antigens from known pandemic strains and from strains that will emerge over time, either through viral strain evolution or from non-human animal reservoirs, such as, but not limited to swine. In one embodiment, the pandemic influenza A subtype H1 hemagglutinin antigen is an H1 HA from a 1918, 1976, or 2009 pandemic influenza A subtype H1 strain. Examples of such pandemic influenza A subtype H1 hemagglutinin antigens include, but are not limited to: A/California/04/2009 (H1N1) HA, A/South Carolina/1/1918 (H1N1) HA, A/Ancona/05/2009, A/California/07/2009 (H1N1) HA, A/Canada-MB/RV2013/2009 (H1N1) HA, A Japan/1070/2009 (H1N1) HA, A/Mexicao/InDRE4114/2009 (H1N1) HA, A/Nanjing/1/2009 (H1N1) HA, A/New York/18/2009 (H1N1) HA, A/Paris/2722/2009 (H1N1) HA, A/Perth/29/2009 (H1N1) HA, A/Sao Paulo/43812/2009 (H1N1) HA, A/Stockholm/31/2009 (H1N1) HA, A/Texas/05/2009 (H1N1) HA, A/New Jersey/1976 (H1N1) HA, A/New Jersey/8/1976 (H1N1) HA, and A/New Jersey/11/1976 (H1N1) HA. In one embodiment, the pandemic influenza A subtype H1 hemagglutinin antigen is A/California/04/2009 (H1N1) HA or A/South Carolina/1/1918 (H1N1) HA. In one embodiment, the antigen is A/California/04/2009 (H1N1) HA. In one embodiment, the antigen is A/South Carolina/1/1918 (H1N1) HA.

One embodiment of the disclosure is an immunogenic protein that comprises at least one epitope of a RBD-A region that lacks any N-linked glycosylation site that is present in the RBD-A region of a non-pandemic influenza A subtype H1 hemagglutinin antigen. Such an N-linked glycosylation site can be any N-linked glycosylation site of a RBD-A region of a non-pandemic influenza A subtype H1 virus. An N-linked glycosylation site can be, but need not be, selected from at least one of the following: (a) an N-linked glycosylation site corresponding to amino acid position 142 of SEQ ID NO:3; (b) an N-linked glycosylation site corresponding to amino acid position 144 of SEQ ID NO:3; (c) an N-linked glycosylation site corresponding to amino acid position 172 of SEQ ID NO:3; (d) an N-linked glycosylation site corresponding to amino acid position 177 of SEQ ID NO:3; (e) an N-linked glycosylation site corresponding to amino acid position 179 of SEQ ID NO:3; and (f) an N-linked glycosylation site corresponding to amino acid position 136 of SEQ ID NO:3. As noted above, the term N-linked glycosylation site refers to the asparagine attachment site, even though the respective RBD-A region has the entire three-amino acid motif. It is also to be appreciated that SEQ ID NO:3 does not include any glycosylation sites in the RBD-A motif because that amino acid sequence represents the hemagglutinin protein of a pandemic strain; this SEQ ID NO is used simply for reference (i.e., 1918 numbering). In addition, the cited amino acid positions represent those in a full-length hemagglutinin protein although a hemagglutinin antigen of the disclosure need not comprise a full-length hemagglutinin protein.

In one embodiment, the immunogenic protein comprises at least one of the following regions: (a) amino acids 131-143 from SEQ ID NO:3 or SEQ ID NO:62; (b) amino acids 170-182 from SEQ ID NO:3 or SEQ ID NO:62; or (c) amino acids 131-146 from SEQ ID NO:3 or SEQ ID NO:62. In one embodiment, the immunogenic protein comprises (a) amino acids 131-143 from SEQ ID NO:3 or SEQ ID NO:62; (b) amino acids 170-182 from SEQ ID NO:3 or SEQ ID NO:62; (c) amino acids 205-215 from SEQ ID NO:3 or SEQ ID NO:62; and (d) amino acids 257-262 from SEQ ID NO:3 or SEQ ID NO:62. In one embodiment, the immunogenic protein comprises (a) amino acids 131-143 from SEQ ID NO:3; (b) amino acids 170-182 from SEQ ID NO:3; (c) amino acids 205-215 from SEQ ID NO:3; and (d) amino acids 257-262 from SEQ ID NO:3. In one embodiment, the immunogenic protein comprises (a) amino acids 131-143 from SEQ ID NO:62; (b) amino acids 170-182 from SEQ ID NO:62; (c) amino acids 205-215 from SEQ ID NO:62; and (d) amino acids 257-262 from SEQ ID NO:62.

In one embodiment, the immunogenic protein comprises at least one epitope from the RBD-A region of a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ JD NO:61, and SEQ ID NO:62. In one embodiment, the immunogenic protein comprises at least one epitope from the RBD-A region of a hemagglutinin antigen comprising an amino acid sequence selected from the group consisting of SEQ ID NO:3, SEQ ID NO:7, SEQ ID NO:19, SEQ ID NO:49, and SEQ ID NO:62. In one embodiment, the immunogenic protein comprises at least one epitope from the RBD-A region of a hemagglutinin antigen comprising amino acid sequence SEQ ID NO:3. In one embodiment, the immunogenic protein comprises at least one epitope from the RBD-A region of a hemagglutinin antigen comprising amino acid sequence SEQ ID NO:62.

In one embodiment, the immunogenic protein comprises the RBD-A region of a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62. In one embodiment, the immunogenic protein comprises the receptor binding domain of a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62. In one embodiment, the immunogenic protein comprises the HA1 polypeptide of a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62. In one embodiment, the immunogenic protein comprises a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62. In one embodiment, the immunogenic protein comprises a hemagglutinin antigen comprising an amino acid sequence selected from the group consisting of SEQ ID NO:3, SEQ ID NO:7, SEQ ID NO:19, SEQ ID NO:49, and SEQ ID NO:62. In one embodiment, the immunogenic protein comprises a hemagglutinin antigen comprising amino acid sequence SEQ ID NO:3. In one embodiment, the immunogenic protein comprises a hemagglutinin antigen comprising amino acid sequence SEQ ID NO:62.

In one embodiment, the immunogenic protein comprises a hemagglutinin antigen encoded by a nucleic acid molecule encoding a protein comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62, or an epitope thereof.

In one embodiment the nucleic acid molecule encoding an immunogenic protein of the disclosure encodes a A/South Carolina/1/1918 (H1N1) HA or A/California/02/2009 (H1N1) HA. In one embodiment the nucleic acid molecule comprises at least one of the following nucleic acid sequences: SEQ ID NO:2, SEQ ID NO:6, SEQ ID NO:18, or SEQ ID NO:50. In one embodiment the nucleic acid molecule comprises SEQ ID NO:2. In one embodiment the nucleic acid molecule comprises SEQ ID NO:50.

In one embodiment, the nucleic acid construct comprises a DNA plasmid that is operatively linked to a nucleic acid molecule encoding at least one of the immunogenic proteins disclosed herein, such that the nucleic acid molecule expresses the protein. In one embodiment, the DNA plasmid comprises a CMV plasmid, such as CMV/R or CMV/R 8 kb. In one embodiment, the nucleic acid construct comprises a CMV/R plasmid operatively linked to a nucleic acid molecule encoding such immunogenic protein. In one embodiment, the nucleic acid construct comprises a CMV/R 8 kb plasmid operatively linked to a nucleic acid molecule encoding such immunogenic protein.

One embodiment is an immunogen comprising a nucleic acid construct having nucleic acid sequence SEQ ID NO:1 (VRC 7730), SEQ ID NO:5 (VRC 7733), SEQ ID NO:17 (VRC 7764), or SEQ ID NO:63 (VRC 9328). In one embodiment, the nucleic acid construct comprises VRC 9328.

Another embodiment of the disclosure is a cross-protective pandemic immunogen comprising an immunogenic protein comprising at least one epitope of the receptor binding domain A (RBD-A) region of a pandemic influenza A subtype H1 hemagglutinin antigen, wherein the encoded RBD-A region is lacking any N-linked glycosylation site that is present in the RBD-A region of a non-pandemic influenza A subtype H1 hemagglutinin antigen, wherein the immunogenic protein can elicit a neutralizing antibody immune response against a homologous pandemic influenza A subtype H1 virus strain and against a heterologous pandemic influenza A subtype H1 virus strain. It is to be appreciated that such a protein can comprise any of the immunogenic proteins described above as being encoded by the nucleic acid molecules of those embodiments.

The present disclosure also provides antibodies that neutralize non-glycosylated influenza A subtype H1 RBD-A regions. Such antibodies are produced by administering an immunogenic protein as disclosed herein to an animal and harvesting immune sera or monoclonal antibodies, using techniques known to those skilled in the art. As such, the antibodies can be polyclonal or monoclonal. Such antibodies have utility against pandemic influenza A subtype H1 viruses.

The present disclosure also provides compositions that comprise a cross-protective pandemic immunogen as disclosed herein. Non-limiting examples of such compositions include the following: One embodiment is a composition comprising an immunogen comprising a nucleic acid construct encoding an immunogenic protein as described above. Another embodiment is a composition comprising an immunogenic protein as described above. Another embodiment is a composition comprising a cross-protective pandemic immunogen and another influenza vaccine that protects against influenza virus, such as, but not limited to, a nucleic acid immunogen, a protein immunogen, a subunit immunogen, an inactivated virus immunogen, a subvirion immunogen, or an attenuated virus immunogen. Such a vaccine can be monovalent or multivalent.

In one embodiment, the composition comprises (a) an immunogen comprising a nucleic acid construct comprising a nucleic acid molecule encoding an immunogenic protein as disclosed herein and (b) an immunogen comprising at least one nucleic acid molecule encoding at least one influenza hemagglutinin antigen (HA) selected from the group consisting of influenza A group 1 HA, influenza A group 2 HA, influenza B group HA, and influenza C group HA. In one embodiment, the composition comprises (a) an immunogen comprising a nucleic acid construct comprising a nucleic acid molecule encoding an immunogenic protein as disclosed herein and (b) an immunogen comprising at least one hemagglutinin antigen (HA) selected from the group consisting of influenza A group 1 HA, influenza A group 2 HA, influenza B group HA, and influenza C group HA. In one embodiment, the composition comprises (a) an immunogen comprising a nucleic acid construct comprising a nucleic acid molecule encoding an immunogenic protein as disclosed herein, and (b) a seasonal influenza vaccine. In one embodiment, the composition comprises (a) an immunogen comprising a nucleic acid construct comprising a nucleic acid molecule encoding an immunogenic protein as disclosed herein, and (b) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein. In one embodiment, the composition comprises (a) an immunogen comprising a nucleic acid construct comprising a nucleic acid molecule encoding an immunogenic protein as disclosed herein, (b) an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein, and (c) a seasonal influenza vaccine.

The present disclosure provides a method to elicit a neutralizing antibody immune response against an influenza A subtype H1 virus in a subject comprising administering to the subject an immunogen or composition comprising a nucleic acid construct comprising a nucleic acid molecule that encodes an immunogenic protein comprising at least one epitope of the receptor binding domain A (RBD-A) region of a pandemic influenza A subtype H1 hemagglutinin antigen, wherein the encoded RBD-A region is lacking any N-linked glycosylation site that is present in the RBD-A region of a non-pandemic influenza A subtype H1 hemagglutinin antigen, wherein the immunogenic protein can elicit a neutralizing antibody immune response against a homologous pandemic influenza A subtype H1 virus strain and against a heterologous pandemic influenza A subtype H1 virus strain. Examples of suitable immunogens encoding an immunogenic protein and compositions comprising such immunogens are disclosed herein. In one embodiment, such immunogens or compositions elicit a neutralizing antibody immune response against a pandemic influenza A subtype H1 virus.

One embodiment is a method to protect a subject from an influenza A subtype H1 virus, such as a pandemic influenza A subtype H1 virus, by administering to the subject any of the immunogens or compositions comprising a nucleic acid construct comprising a nucleic acid molecule encoding an immunogenic protein of the disclosure. Such protection can be either therapeutic (i.e., to treat an influenza virus infection) or prophylactic (i.e., to protect a subject from disease caused by influenza virus or to prevent or reduce infection by influenza virus). Depending on the nature of the immunogens or compositions, protection against other influenza virus types, groups and/or subtypes can also be achieved.

One embodiment is a method to reduce pandemic influenza A subtype H1 virus in an animal reservoir comprising administering to animals in the reservoir any of the immunogens or compositions comprising a nucleic acid construct comprising a nucleic acid molecule encoding an immunogenic protein of the disclosure. As used herein, an animal reservoir is a species, genus, class or family of animals that harbors influenza A subtype H1 viruses that, when they infect humans, lead to pandemic outbreaks of influenza. A non-limiting example of such animals are swine. In one embodiment the virus in the animal reservoir is reduced to a level such that it is not transmitted to humans. In one embodiment, the virus in the animal reservoir is eradicated.

The present disclosure also provides a method to elicit a neutralizing antibody immune response against a pandemic influenza A subtype H1 virus comprising administering to a subject an immunogen comprising a nucleic acid molecule encoding a pandemic influenza A subtype H1 hemagglutinin antigen (HA), wherein the HA is heterologous to the virus against which an immune response is being elicited, wherein the immunogen elicits the immune response in the patient. In one embodiment, the hemagglutinin antigen lacks any N-linked glycosylation site that is present in the receptor binding domain A (RBD-A) region of a hemagglutinin antigen from a non-pandemic influenza A virus. In one embodiment, the hemagglutinin antigen can be any of the immunogenic proteins disclosed herein. In one embodiment, the method protects the subject against pandemic influenza A subtype H1 virus.

The present disclosure also includes administering a cross-protective pandemic immunogen comprising any of the immunogenic proteins of the embodiments. Such an immunogen will elicit a neutralizing antibody immune response against a pandemic influenza A subtype H1 virus. Compositions of such an immunogenic protein with other immunogens, such as those disclosed herein, also have the potential of eliciting neutralizing antibody immune responses against other influenza virus as well as against a pandemic influenza A subtype H1 virus.

### VRC 9328

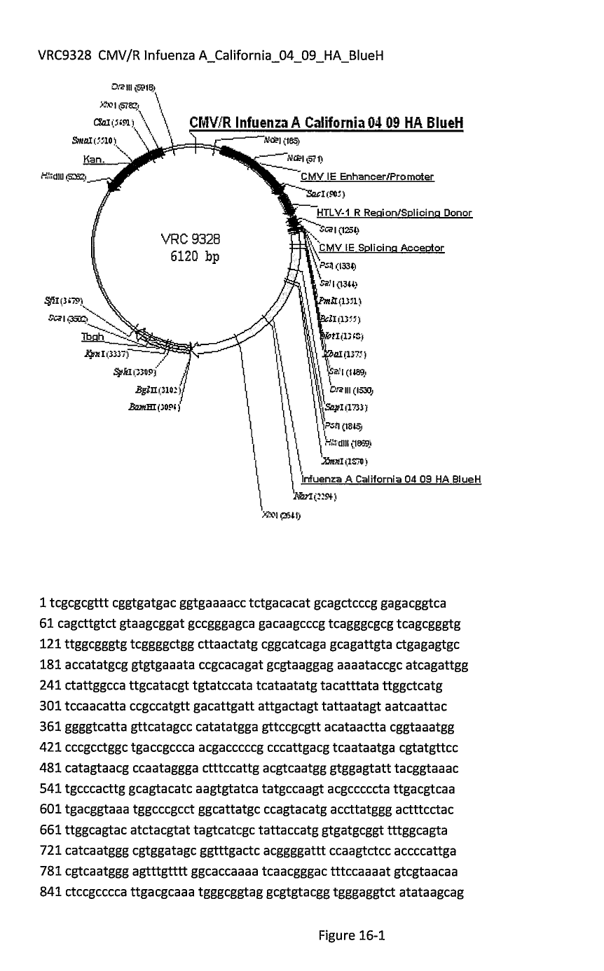

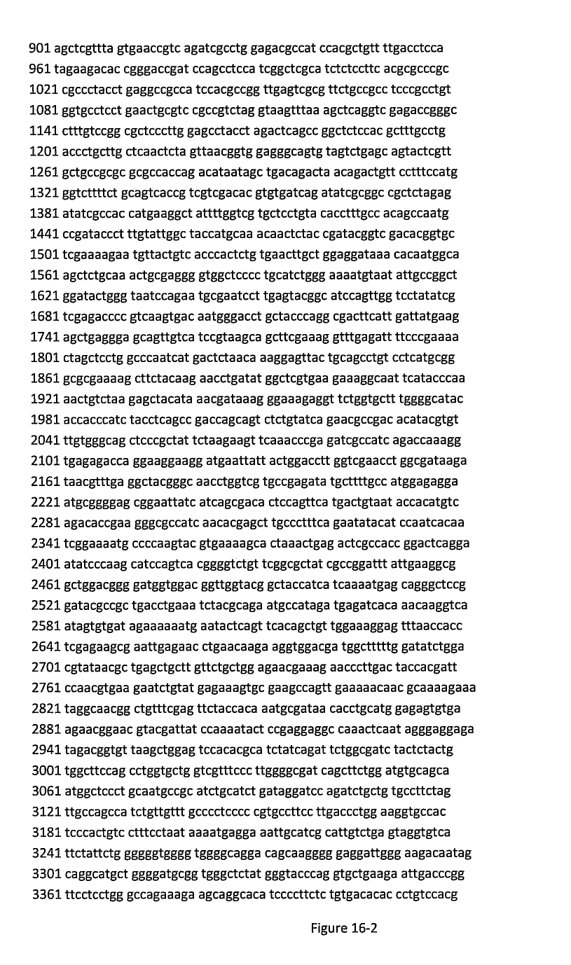

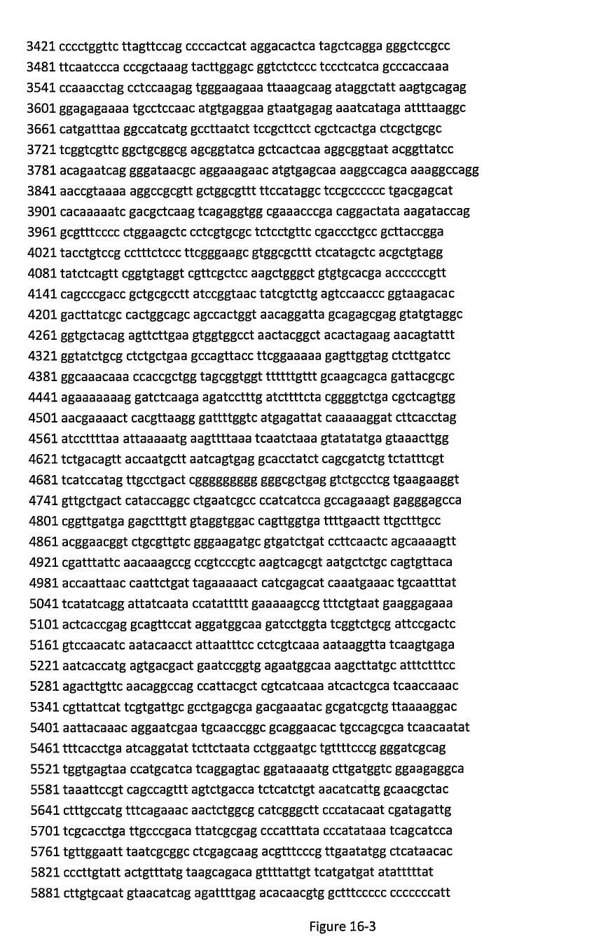

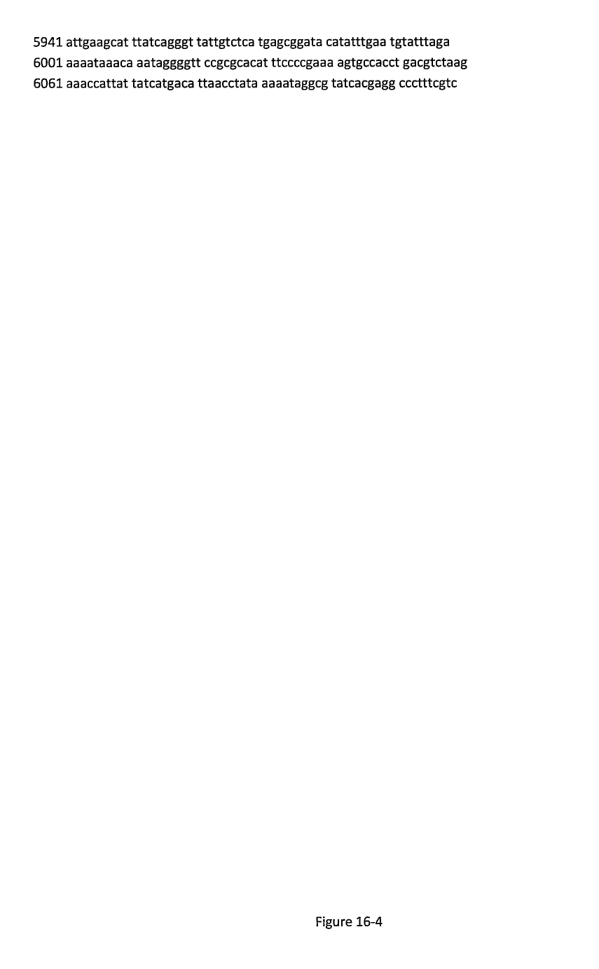

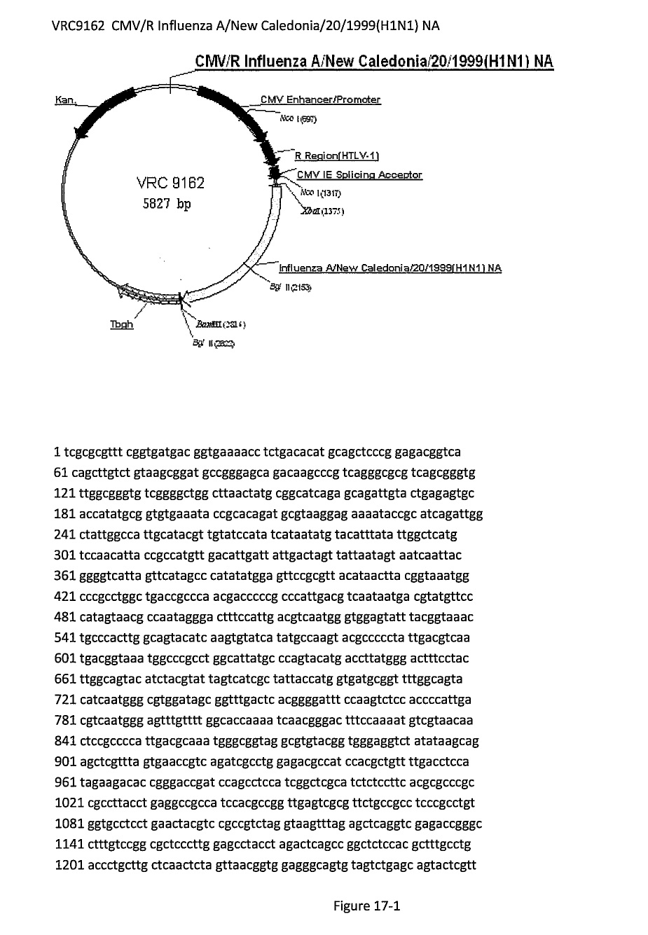

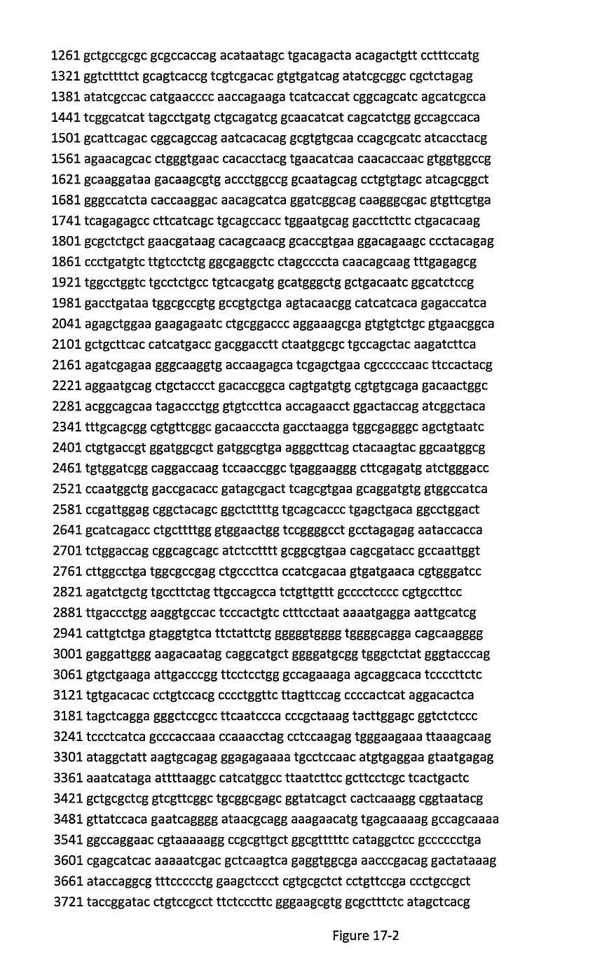

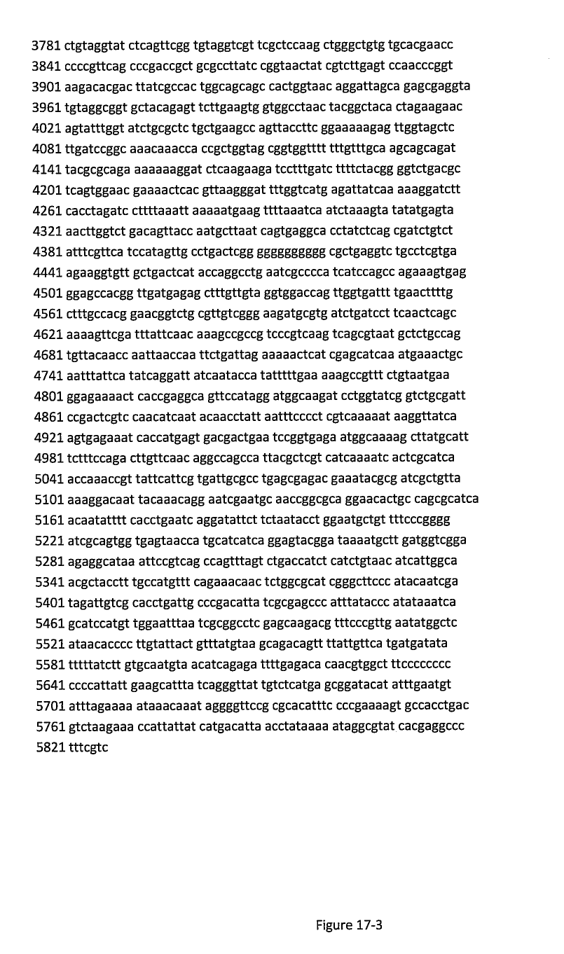

The present disclosure provides an immunogen comprising nucleic acid construct VRC 9328. VRC-9328, the map of which is depicted in FIG. 16, is a CMV/R plasmid that encodes Influenza A/California/04/2009 (H1N1) HA; the BlueH designates the manufacturer of the nucleic acid construct. VRC 9328 elicits a neutralizing antibody immune response against pandemic influenza A subtype H1 strains, including A/South Carolina/1/1918 (H1N1) and A/California/04/2009 (H1N1).

The present disclosure also provides compositions that comprise VRC 9328. Non-limiting examples of such compositions include the following: One embodiment is a composition that comprises VRC 9328 and an immunogen comprising at least one hemagglutinin antigen (HA) selected from the group consisting of influenza A group 1 HA, influenza A group 2 HA, influenza B group HA, and influenza C group HA. One embodiment is a composition that comprises VRC 9328 and a seasonal influenza vaccine. One embodiment is a composition that comprises VRC 9328 and an immunogen comprising a nucleic acid molecule encoding a pandemic H1 HA heterologous to influenza A/California/04/2009 (H1N1) HA. One embodiment is a composition that comprises VRC 9328, a seasonal influenza vaccine, and an immunogen comprising a nucleic acid construct comprising a nucleic acid molecule encoding an influenza A subtype H1 hemagglutinin glycan-shielded receptor binding domain A (RBD-A) region and at least one influenza A subtype H1 hemagglutinin antigenic site as disclosed herein.

The present disclosure also provides a method to elicit a neutralizing antibody immune response against an influenza A subtype H1 virus in a subject comprising administering to the subject any of the disclosed immunogens or compositions comprising VRC 9328. In one embodiment, the influenza A subtype H1 virus is a pandemic influenza A subtype H1 virus. In one embodiment, the influenza A subtype H1 virus is a homologous pandemic influenza A subtype H1 virus. In one embodiment, the influenza A subtype H1 virus is a heterologous pandemic influenza A subtype H1 virus.

### Administration Methods

Immunogens and compositions of the present disclosure can be administered to subjects using techniques known to those skilled in the art; see, for example, WO 2007/100584 A2, published Sep. 7, 2007; WO 2008/112017 A2, published Sep. 18, 2008, WO 2009/092038 A1, published Jul. 23, 2009, and WO 2010/036948, published Apr. 1, 2010, all of which are incorporated by reference herein in their entireties. Such immunogens and compositions can include an excipient, such as a pharmaceutically acceptable excipient. Such immunogens and compositions can also include a carrier or an adjuvant. Routes of administration can be determined by those skilled in the art. Doses can also be determined by those skilled in the art. Such immunogens and compositions can be administered once or several times. Such immunogens and compositions can be administered as a prime and then boosted with the same immunogens and compositions, or with other compositions, such as nucleic acid (e.g., adenoviral or retroviral vectors encoding influenza HAs, pseudotyped lentiviruses encoding influenza HAs), protein, subunit, subvirion, inactivated virus, attenuated virus, seasonal influenza vaccine, or other influenza vaccines.

### Method to Detect Emergence of Non-Pandemic Virus

The present disclosure also provides a method to detect the emergence of a non-pandemic influenza A subtype H1 virus from a pandemic population of influenza A subtype H1 virus, which method comprises (a) isolating a biological sample containing influenza A virus; and (b) testing the hemagglutinin antigen of said virus for the presence of N-linked glycans at positions corresponding to amino acids 136, 142, 144, 172, 177 and 179 of SEQ ID NO:3; wherein the presence of glycan at any of those positions indicates the emergence of a non-pandemic virus. The present disclosure also provides kits to enable such methods. Such a non-pandemic virus can be an evolving or seasonal influenza virus.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations are used.

Nucleic acid constructs encoding different versions of HA proteins (A/South Carolina/1/1918, GenBank AF117241; A/PR/8/1934, GenBank ABD77675; A/New Caledonia/20/1999, GenBank AY289929; and A/California/04/2009, GenBank FJ966082 and NA proteins (A/Brevig Mission/1/1918, GenBank AAF77036; A/New Caledonia/20/1999, GenBank CAD57252; and A/California/04/2009, GenBank FJ966084) were synthesized using human-preferred codons as described (Kong, W.-P. et al. Protective immunity to lethal challenge of the 1918 pandemic influenza virus by vaccination. Proc. Natl. Acad. Sci. USA 103, 15987-15991 (2006)) by GeneArt. The glycosylation site mutations were introduced using the QuikChange Site-Directed Mutagenesis kit (Agilent Technologies).

The recombinant lentiviral vectors expressing a luciferase reporter gene were produced as described (Yang, Z.-Y. et al. Immunization by avian H5 influenza hemagglutinin mutants with altered receptor binding specificity. Science 317, 825-828 (2007)_. For the production of H1N1 pseudoviruses, a human type 2 transmembrane serine protease TMPRSS2 gene was included in transfection for the proteolytic activation of HA (E. Böttcher, T. Matrosovich, M. Beyerle, H. D. Klenk, W. Garten, M. Matrosovich, Proteolytic activation of influenza viruses by serine proteases TMPRSS2 and HAT from human airway epithelium. J. Virol., 80, 9896-9898 (2006)).

Influenza A/California/04/2009 (H1N1) is also referred to herein as influenza A (H1N1) 2009 (CA 04/09), A (H1N1) 2009, A(H1N1) 2009, and 2009 CA.

Influenza A/South Carolina/1/1918 (H1N1) is also referred to herein as A (H1N1) 1918 (SC), H1N1 (1918 SC), and 1918 SC.

Influenza A/New Caledonia/20/1999 is also referred to herein as A (H1N1) 1999 (NC), H1N1 1999 (New Caledonia), and 1999 NC.

### Example 1

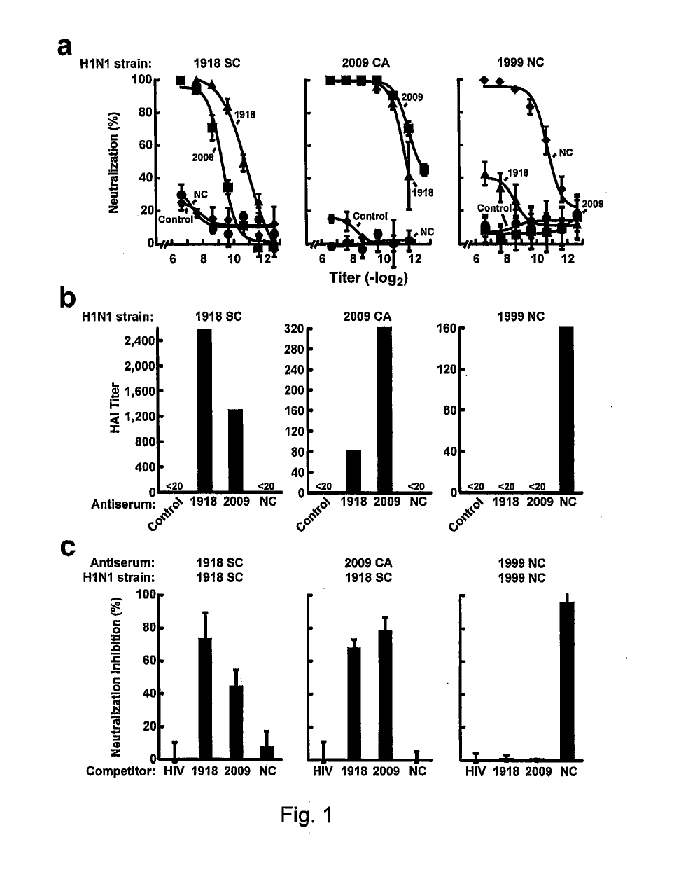

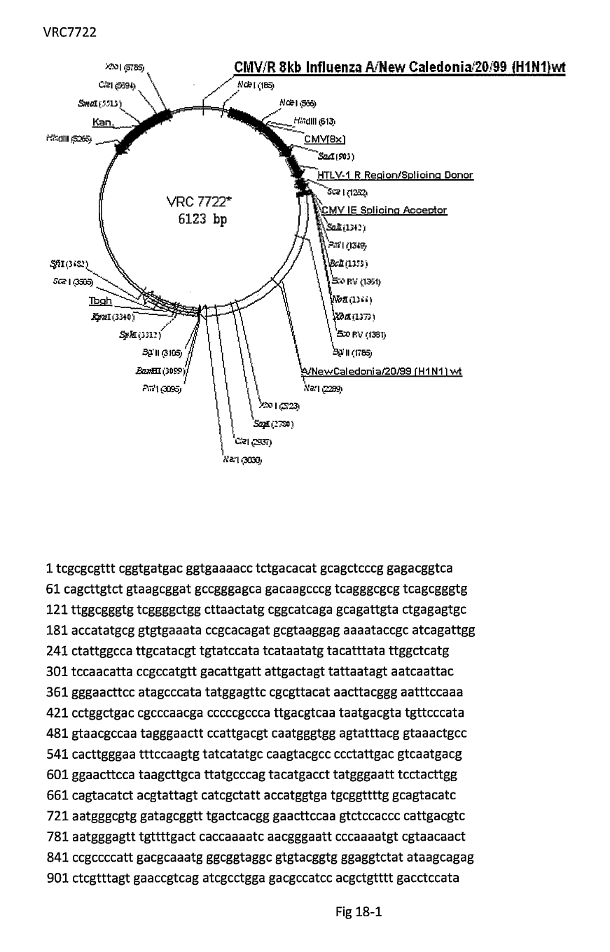

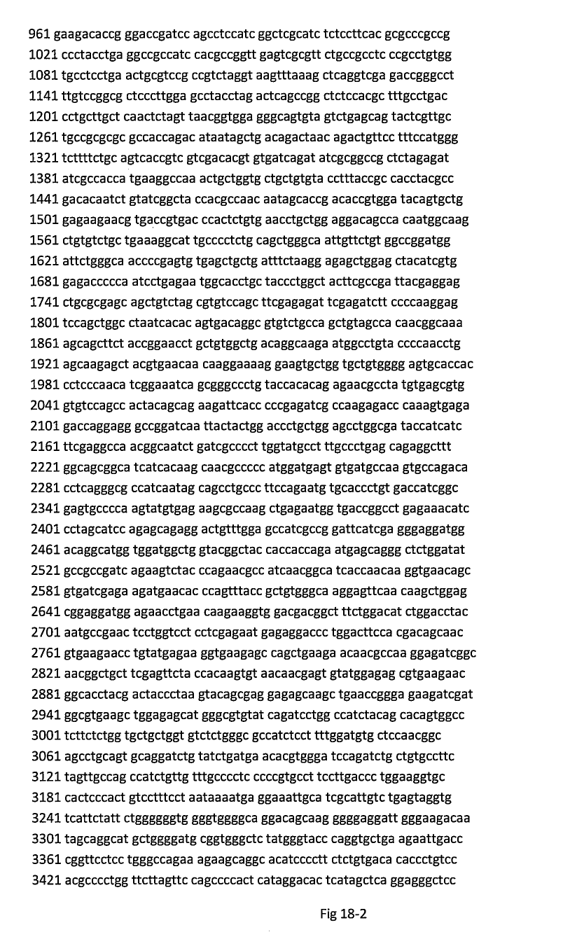

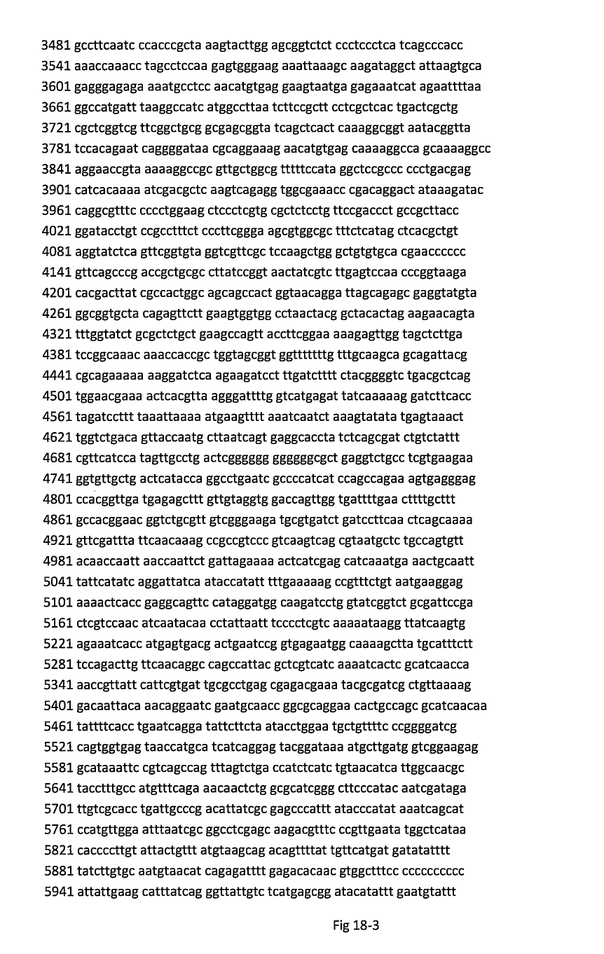

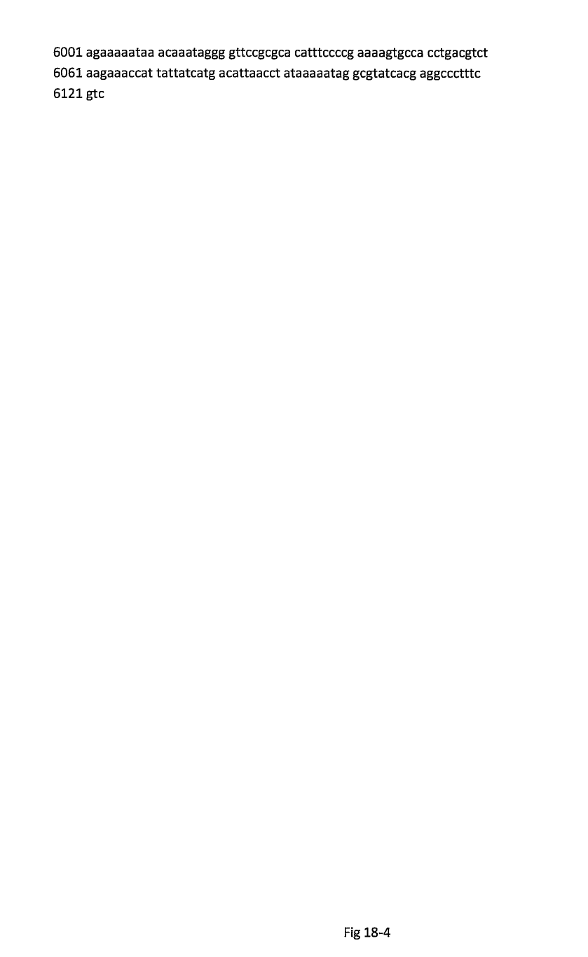

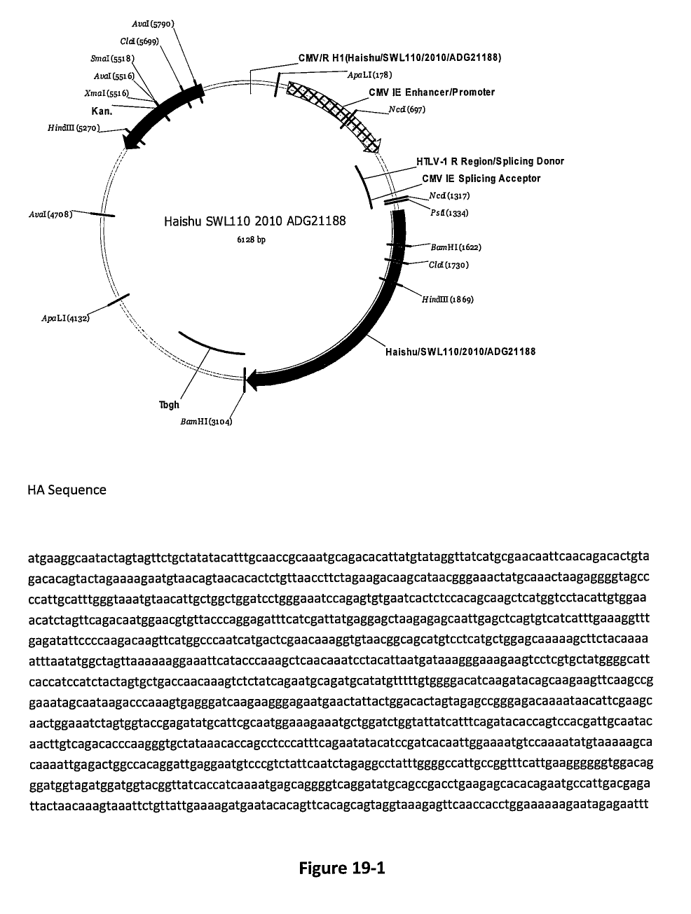

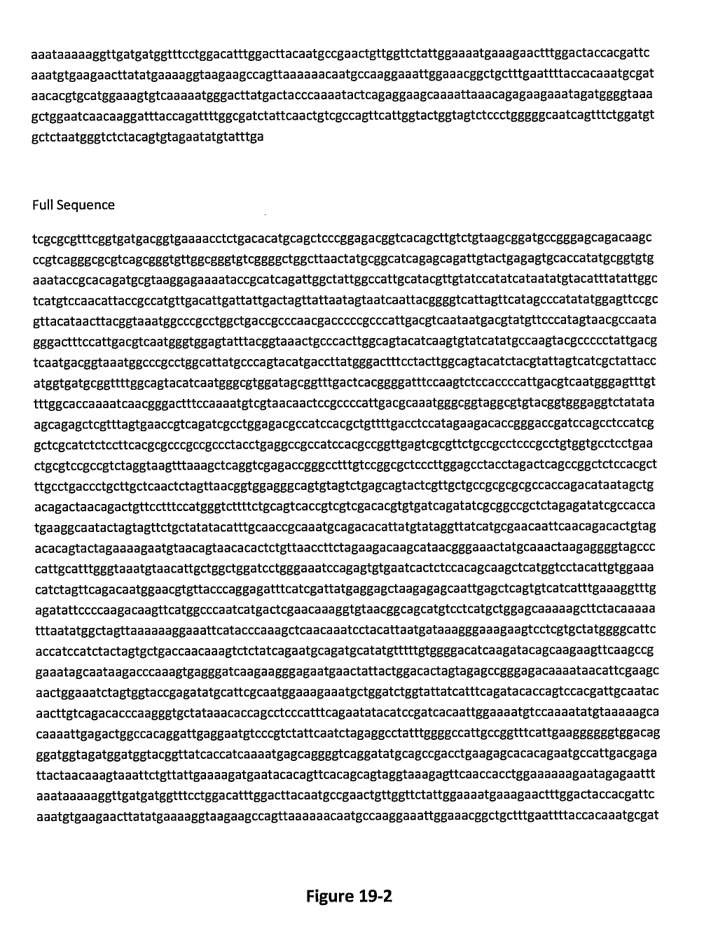

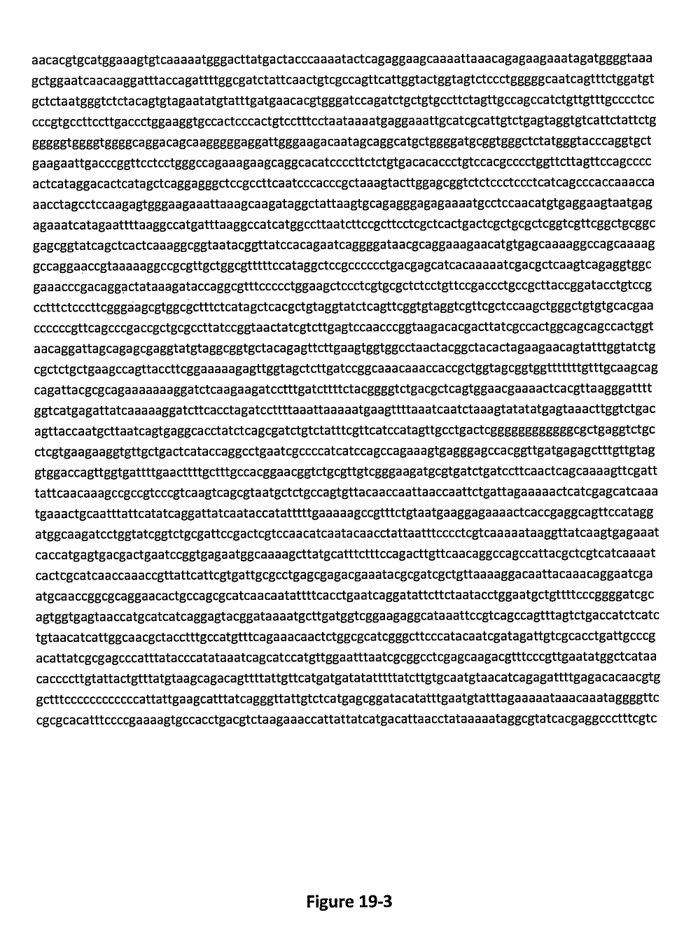

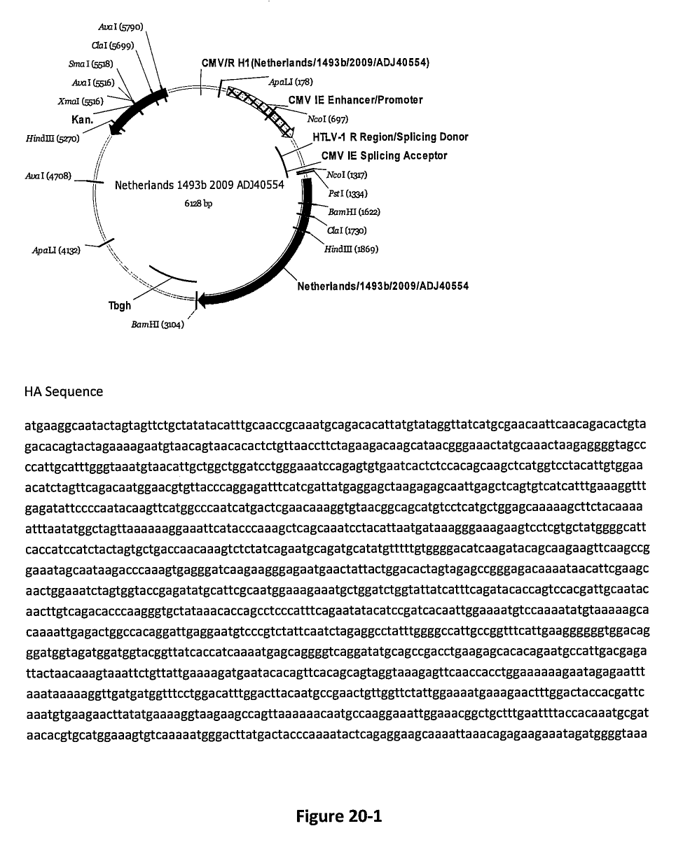

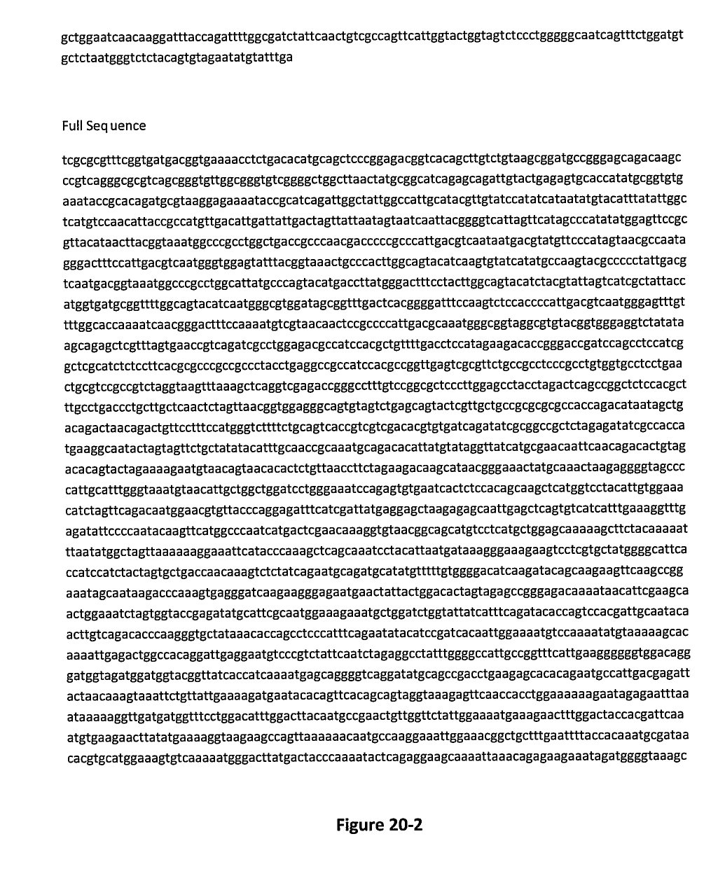

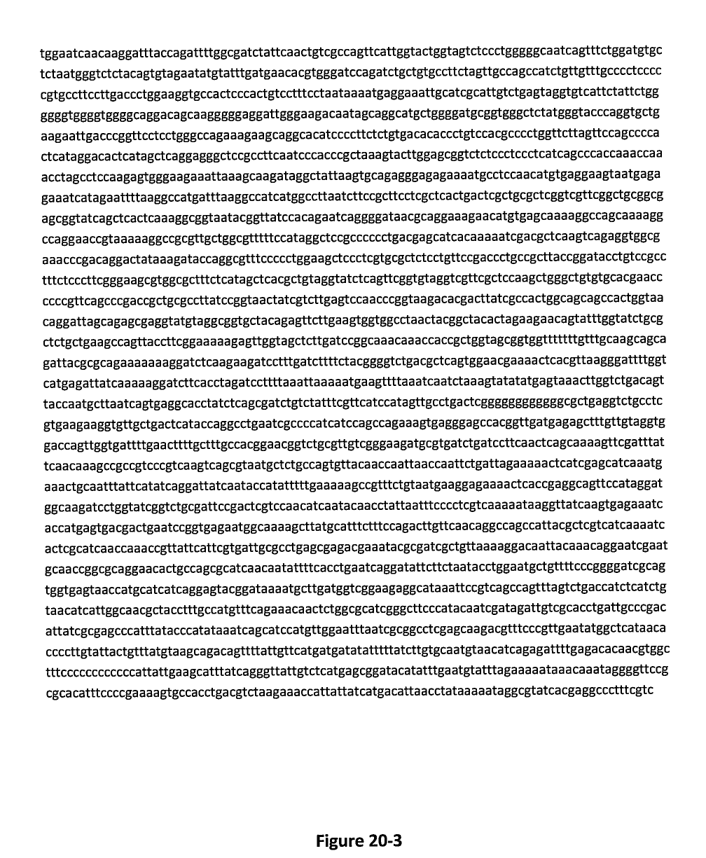

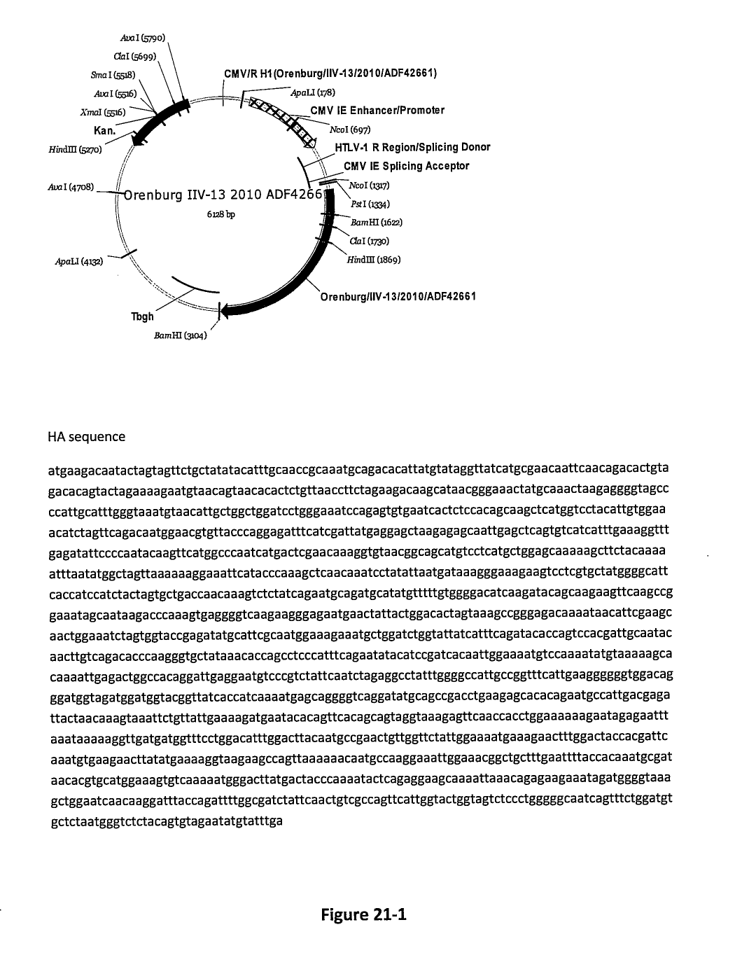

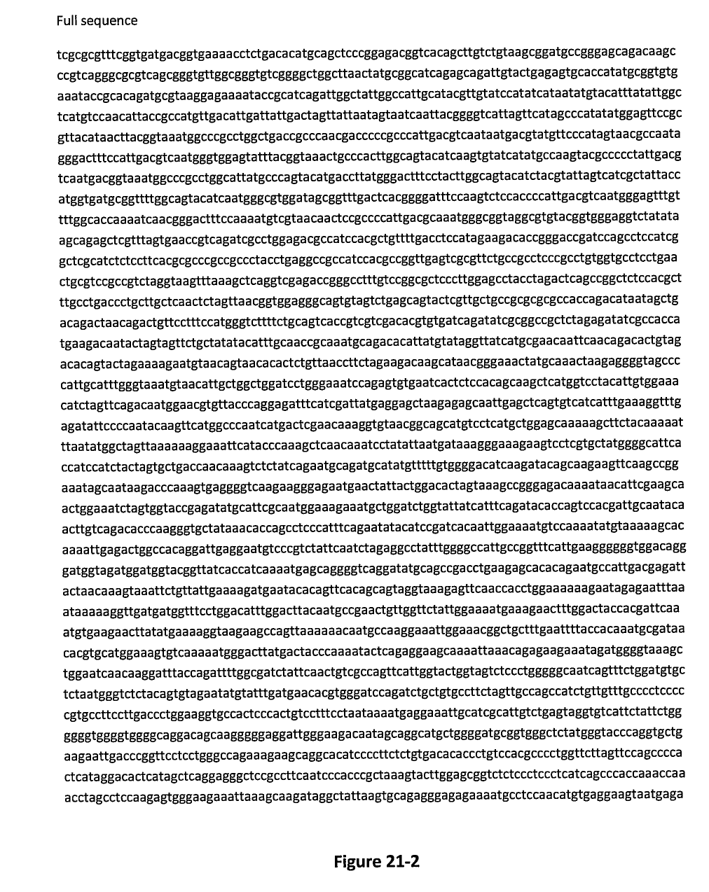

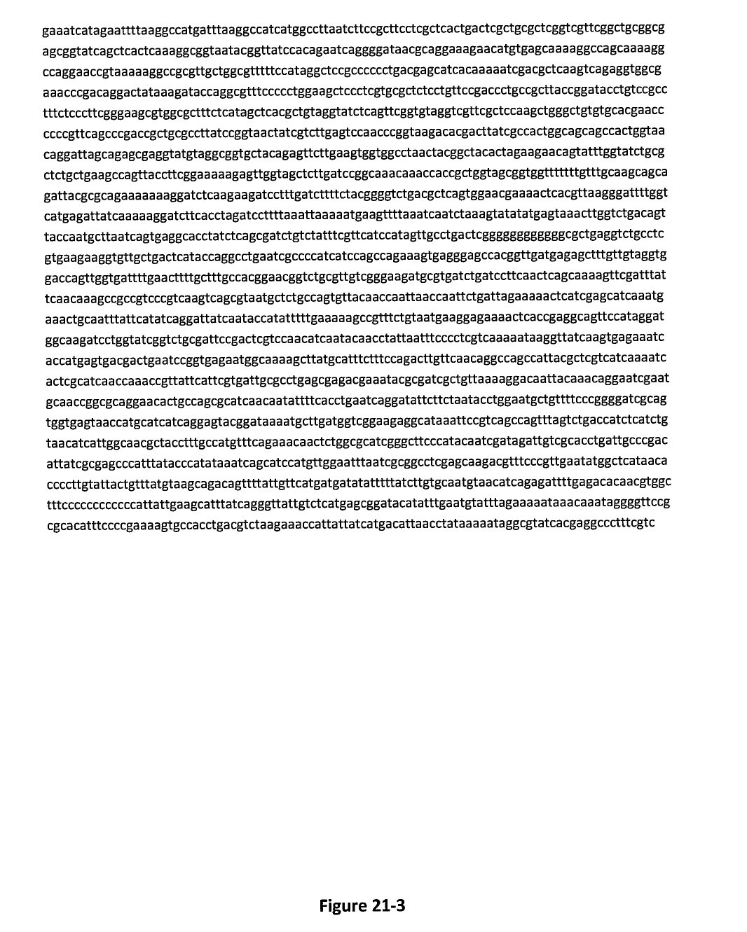

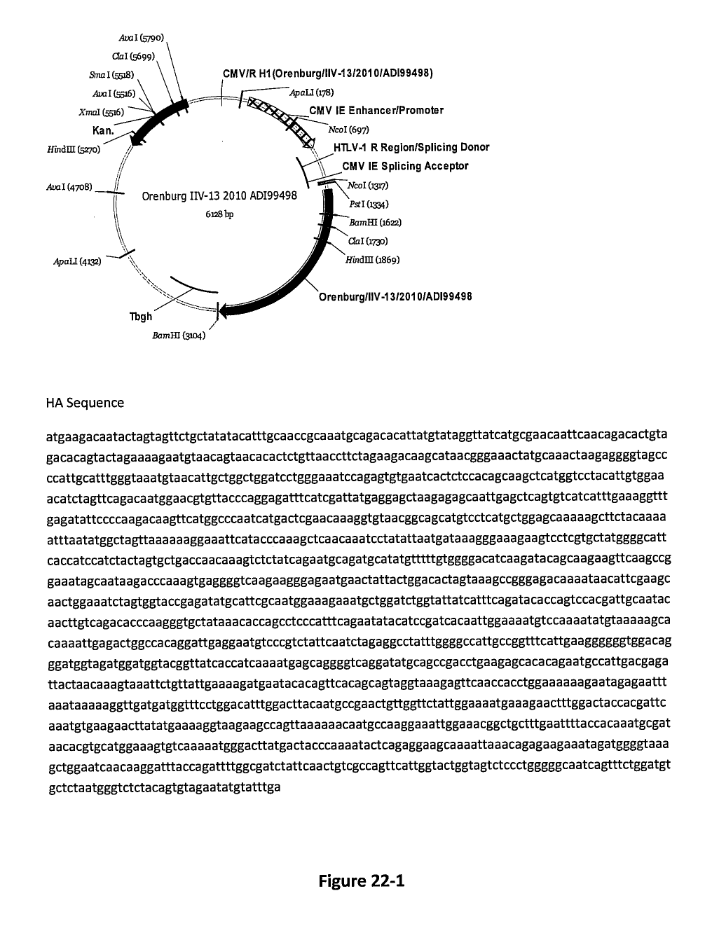

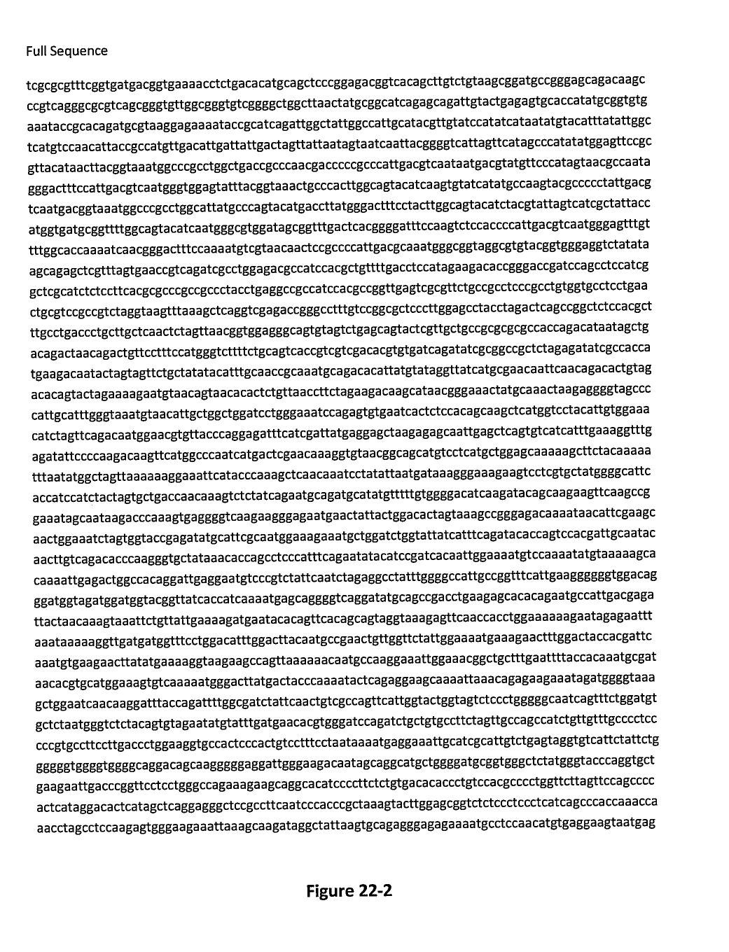

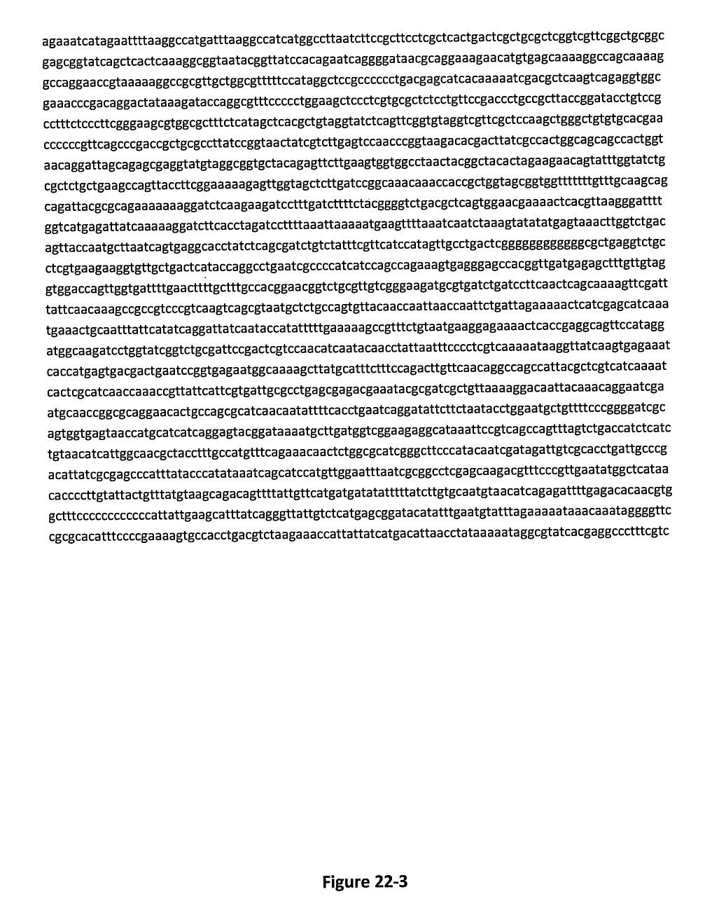

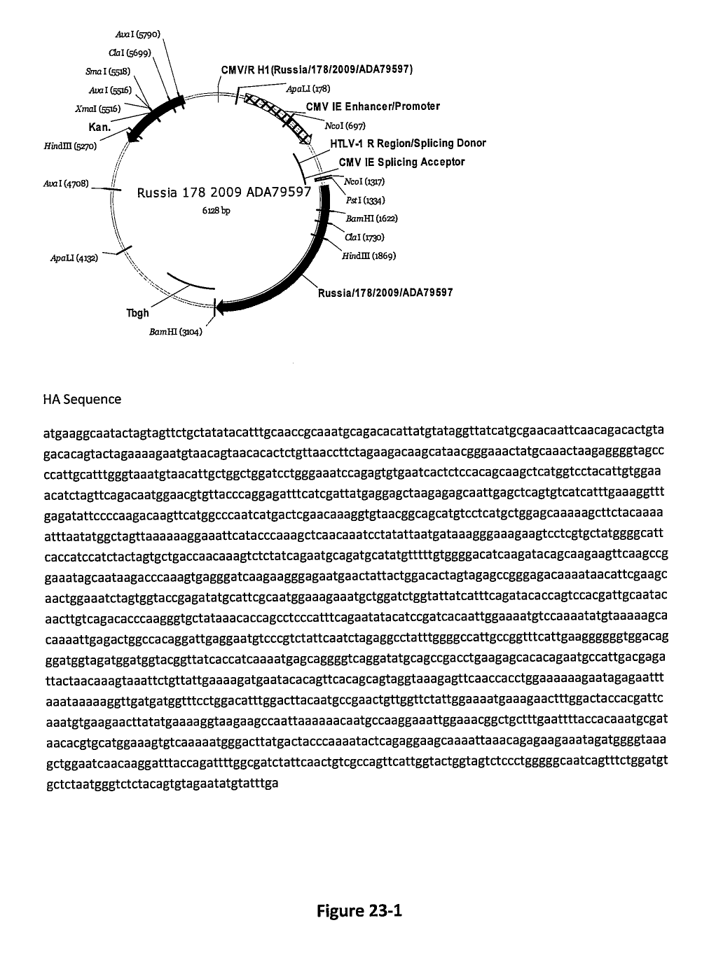

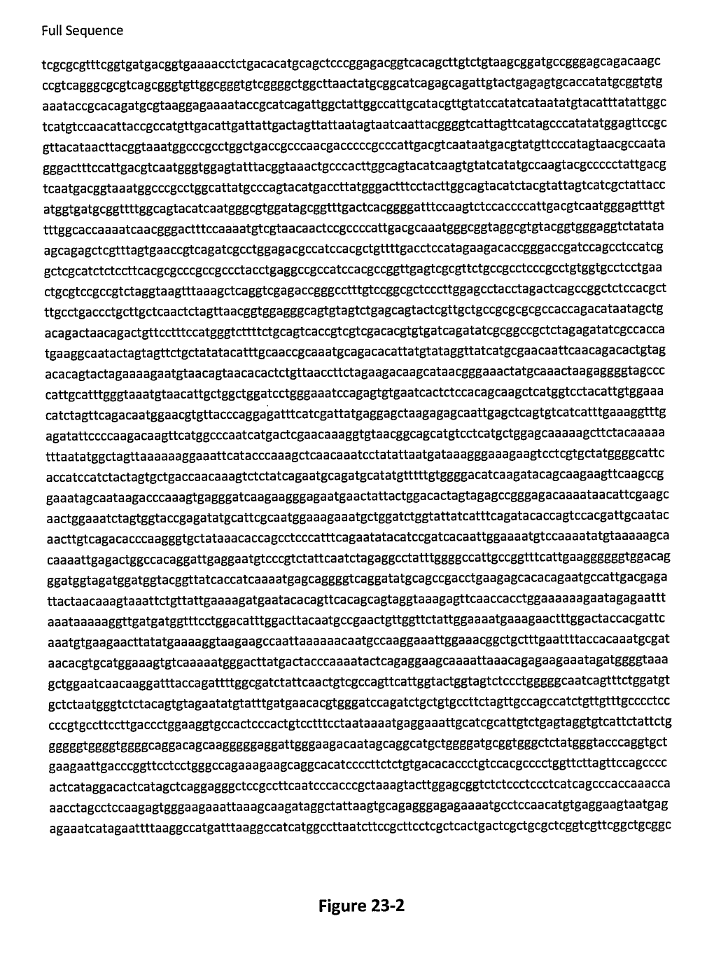

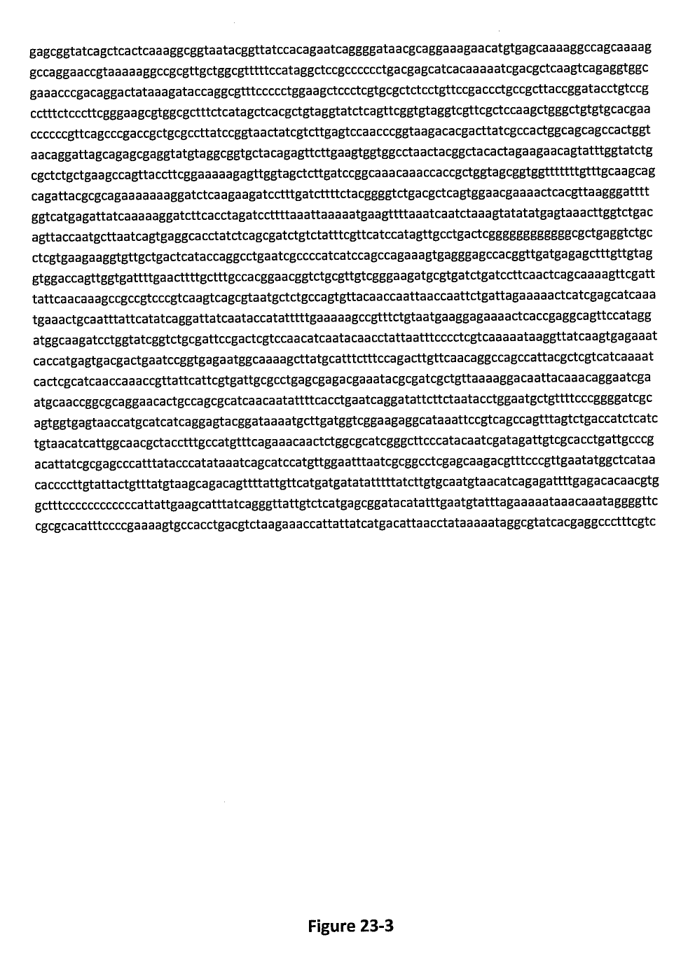

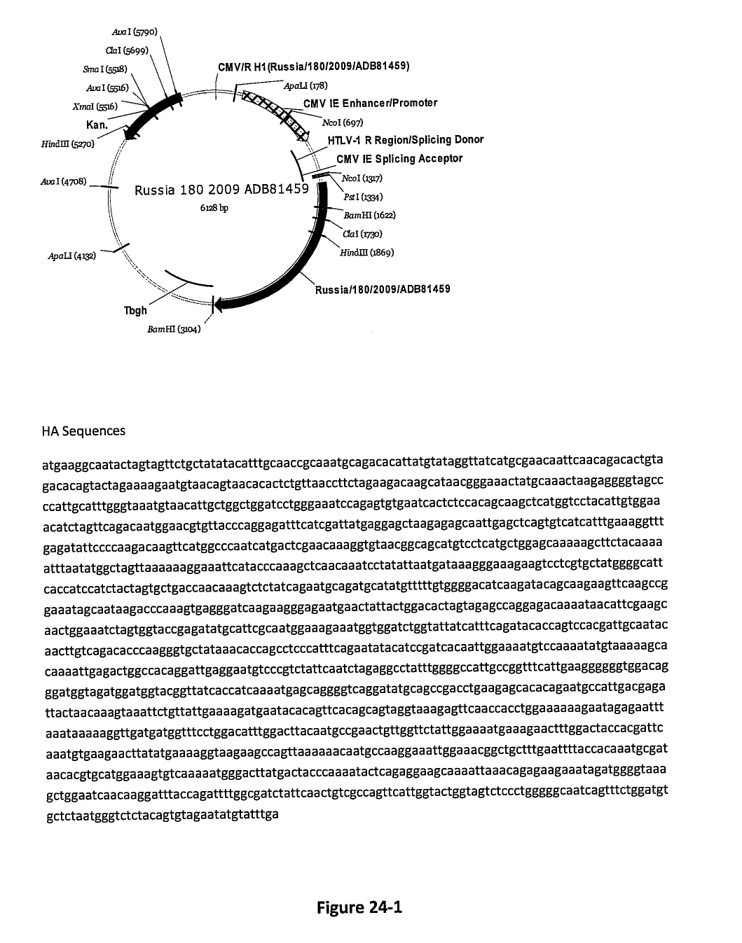

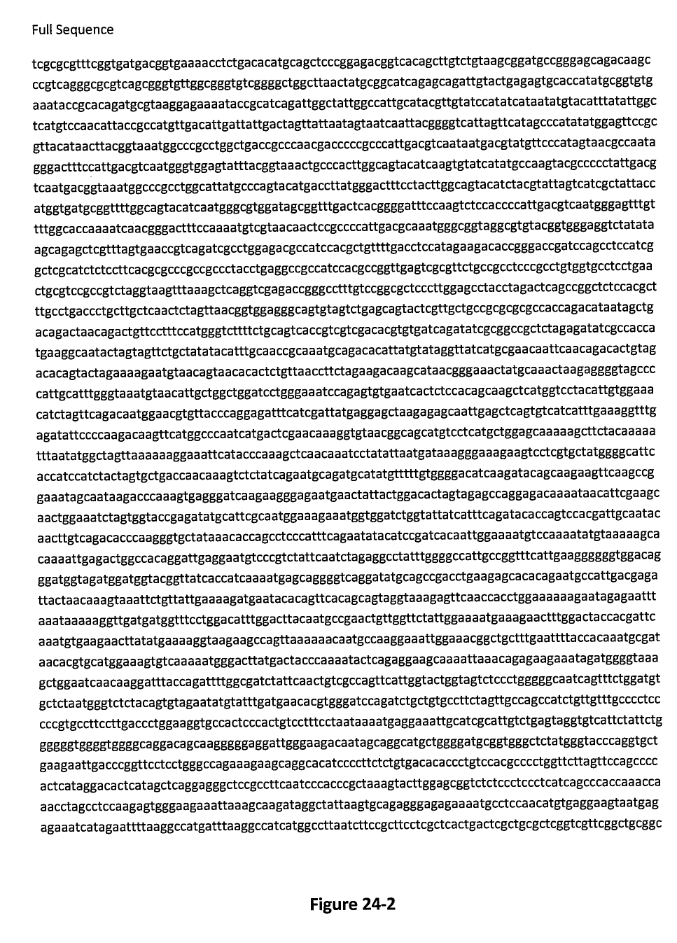

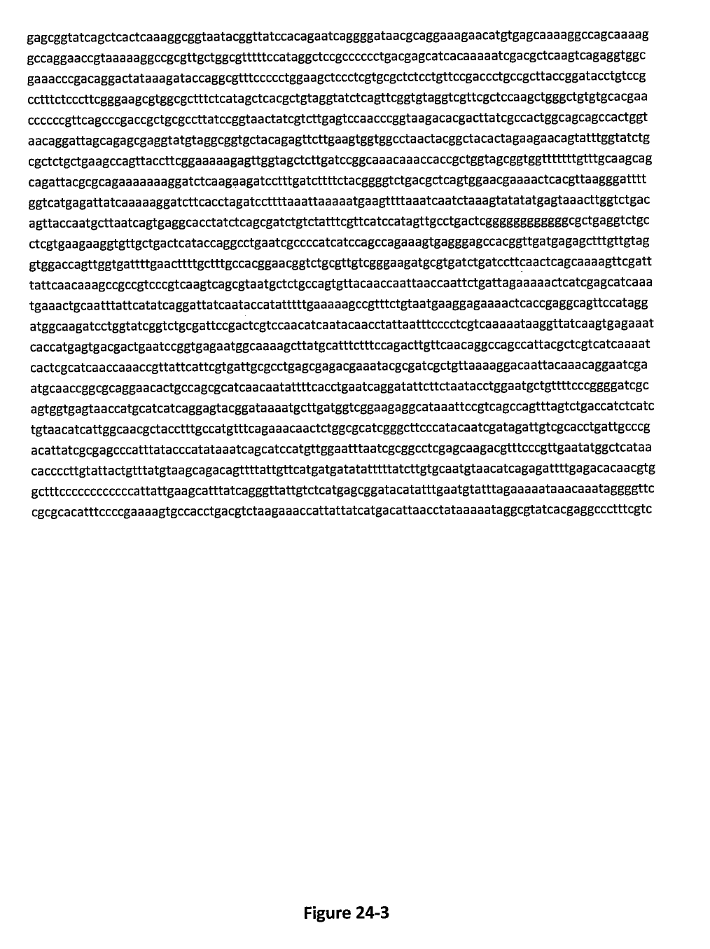

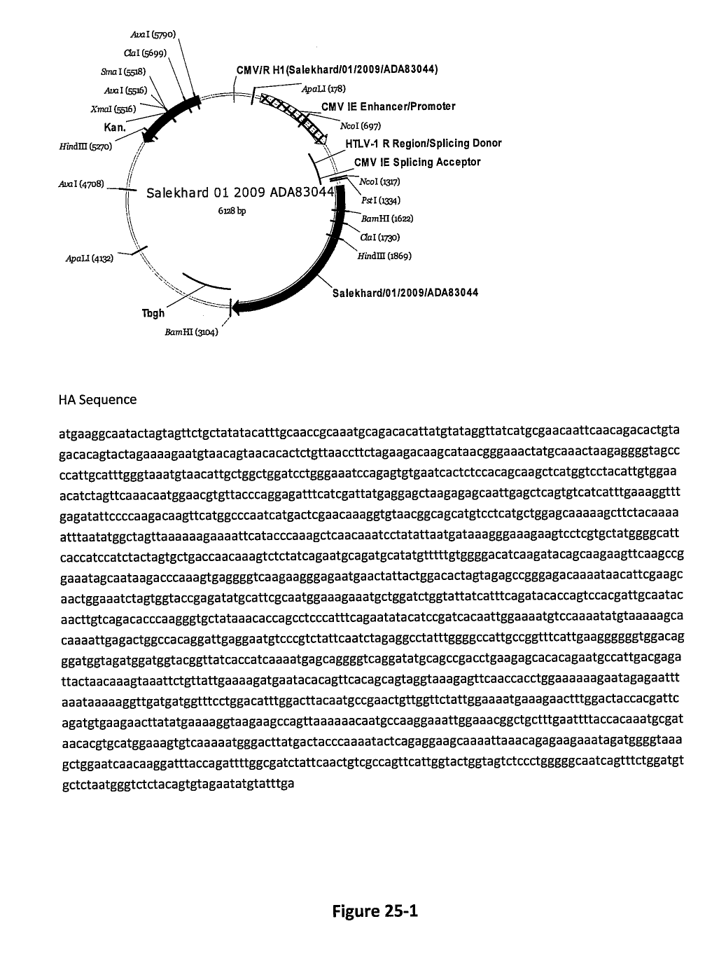

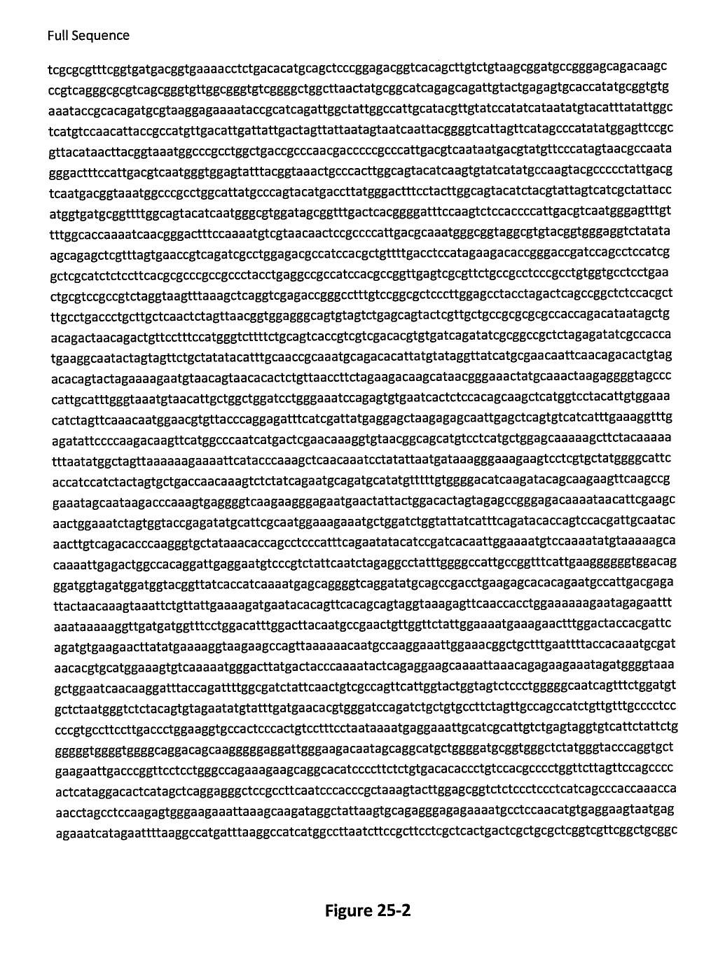

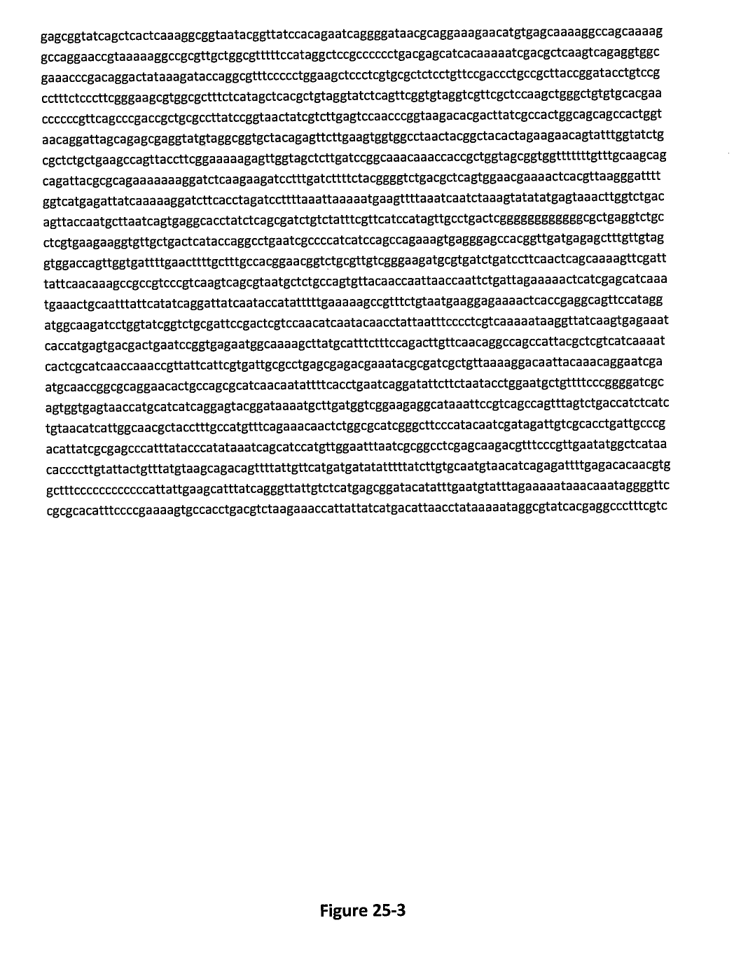

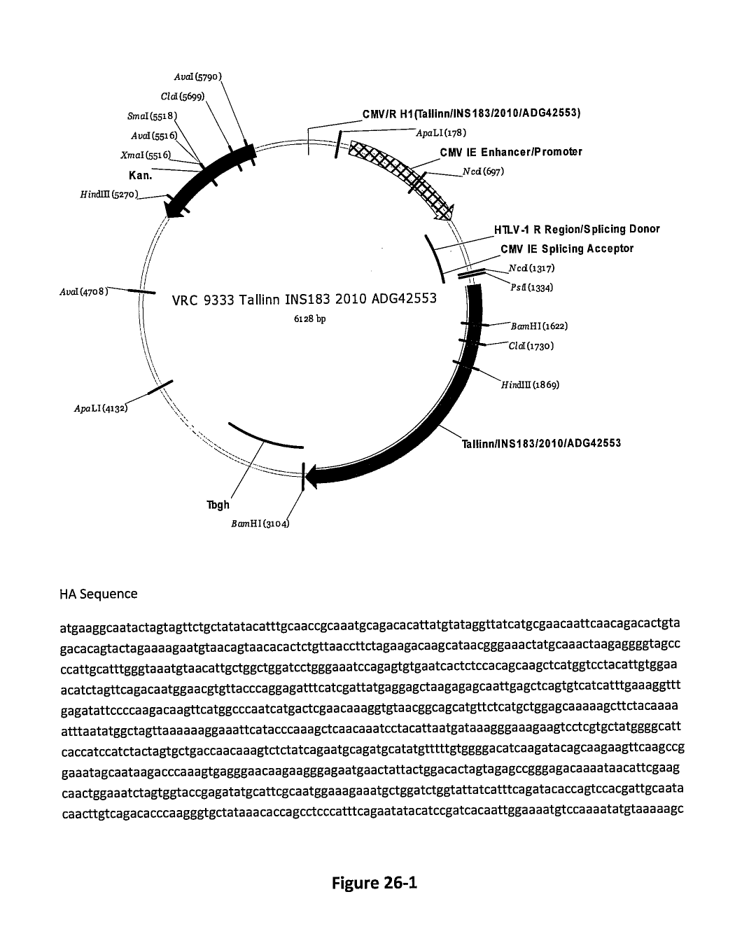

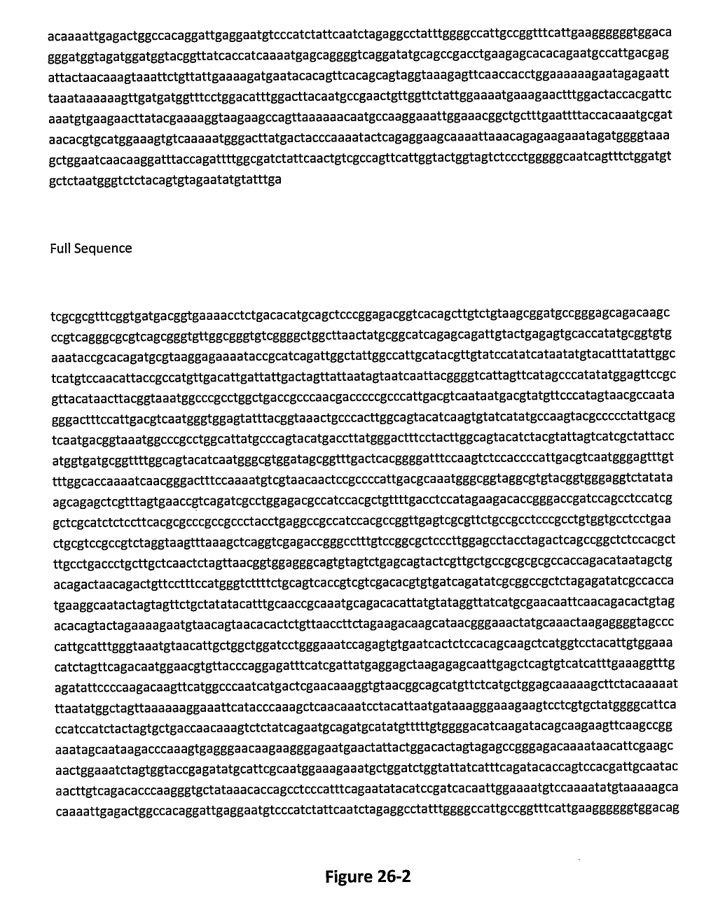

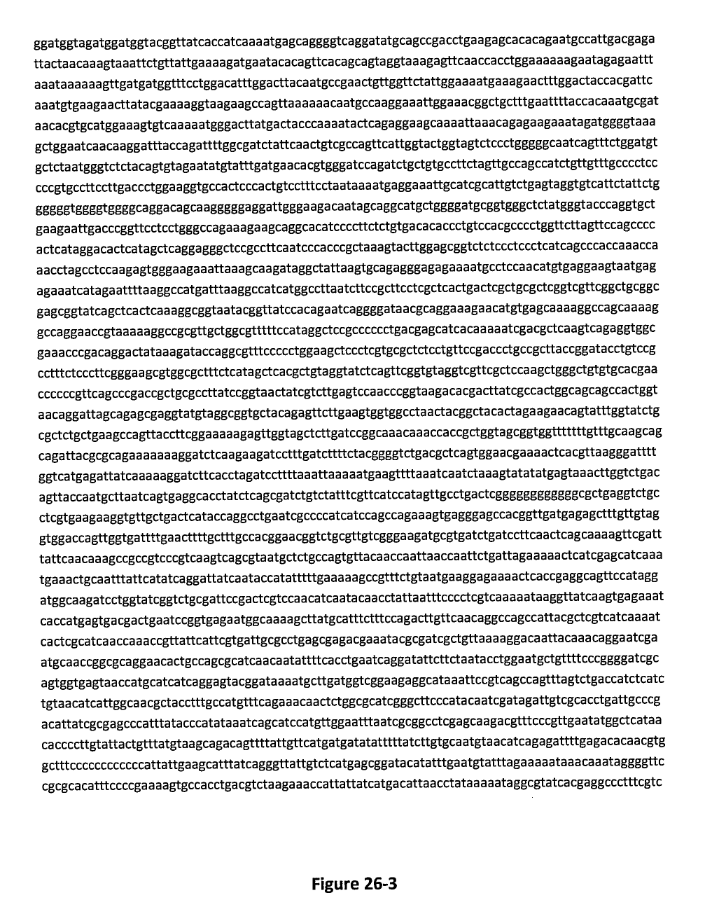

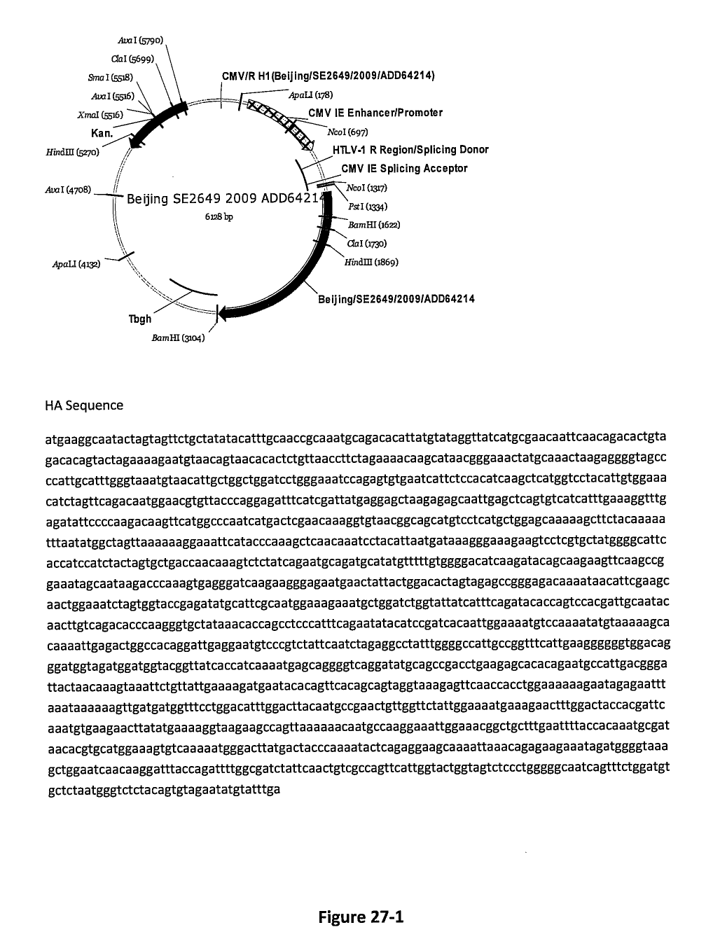

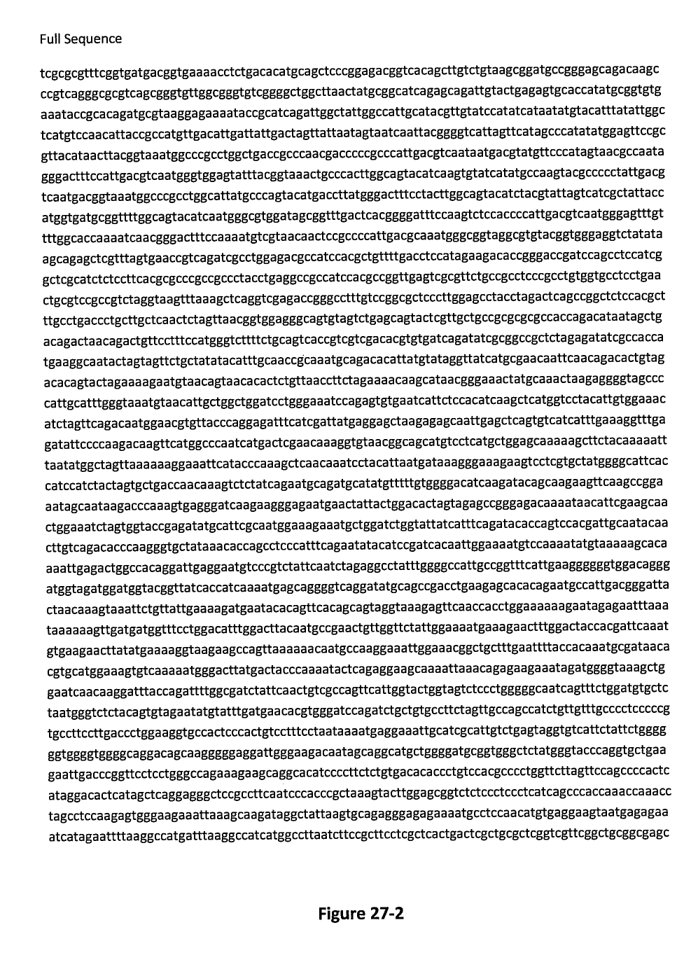

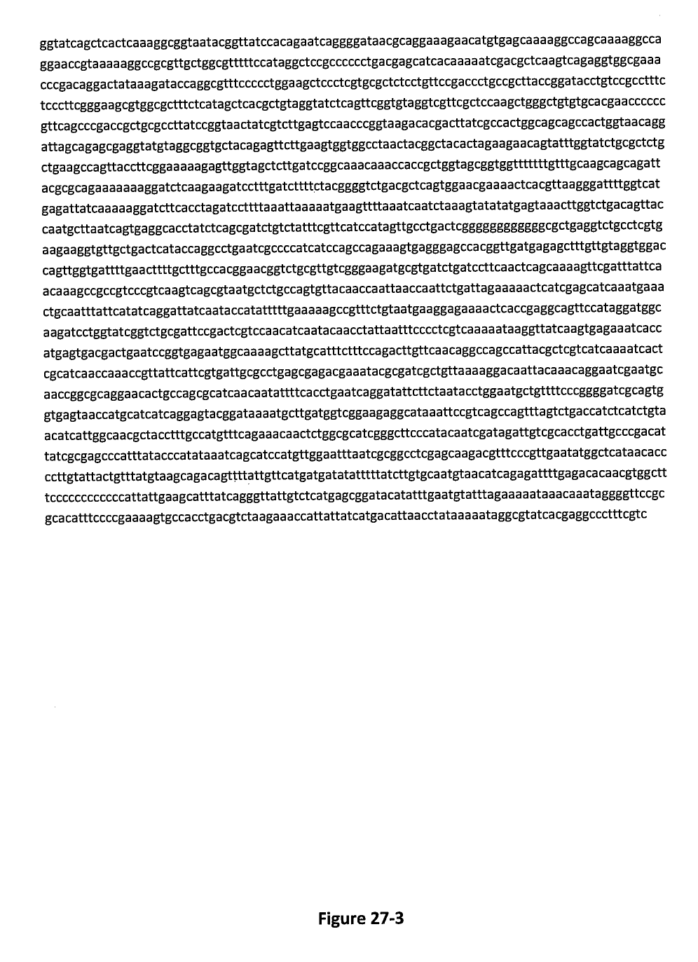

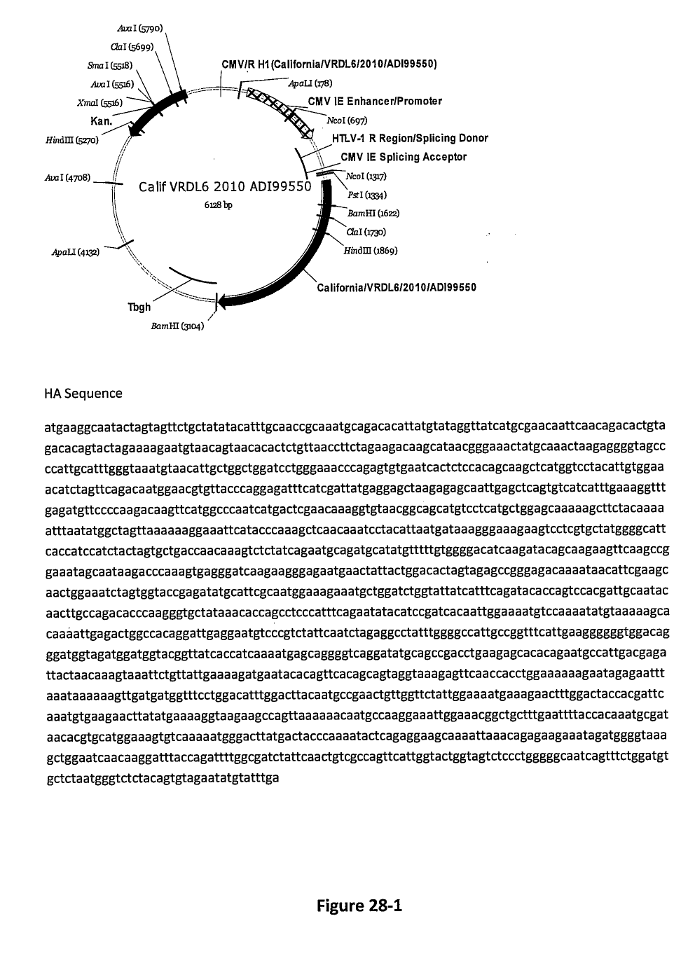

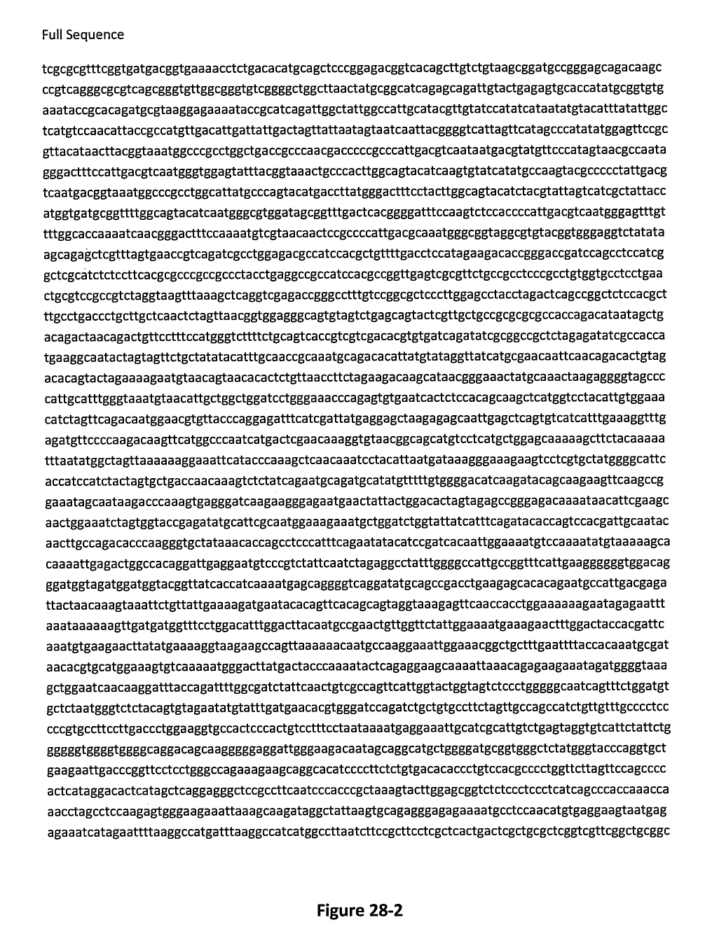

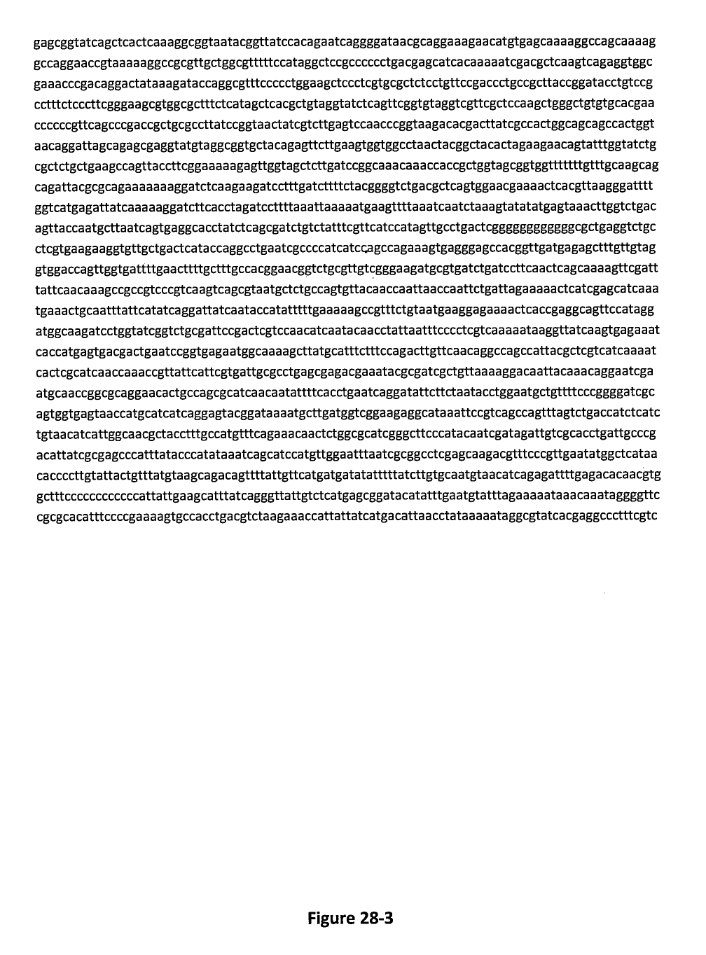

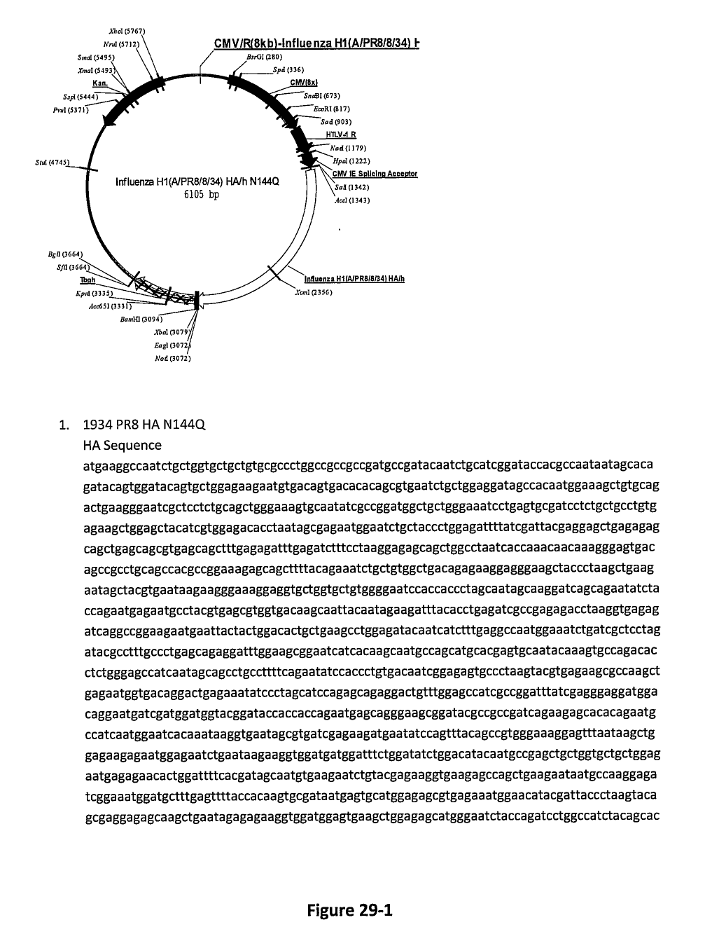

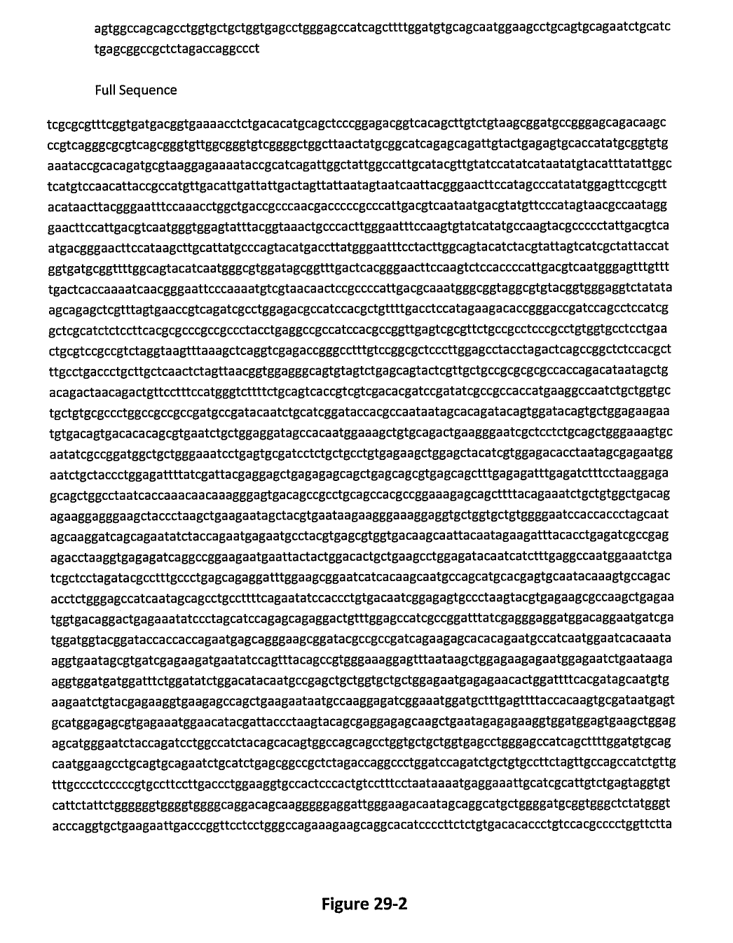

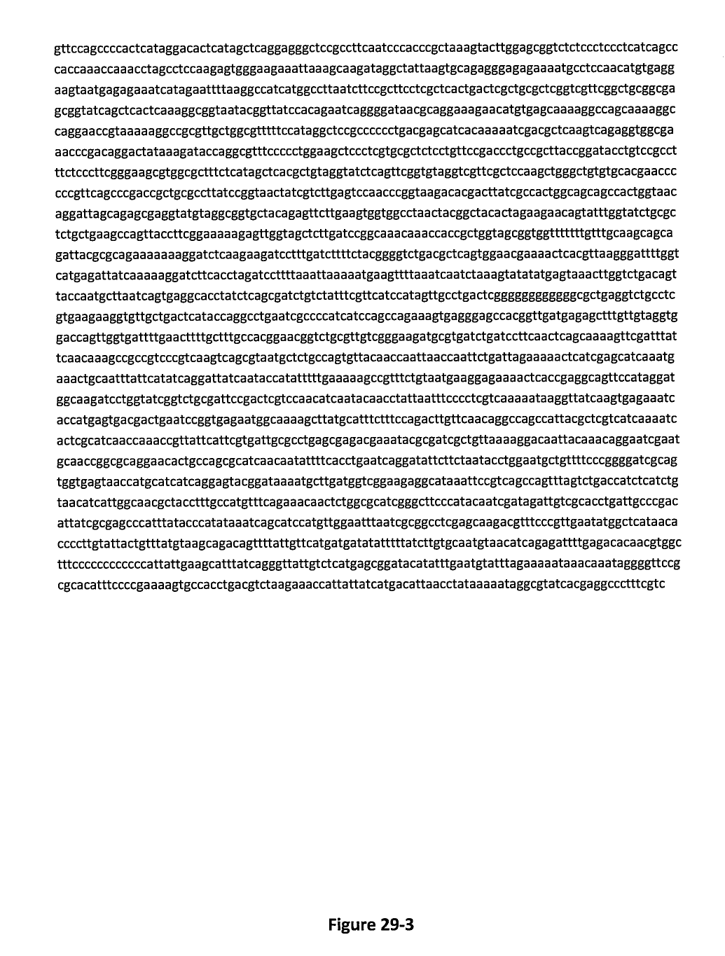

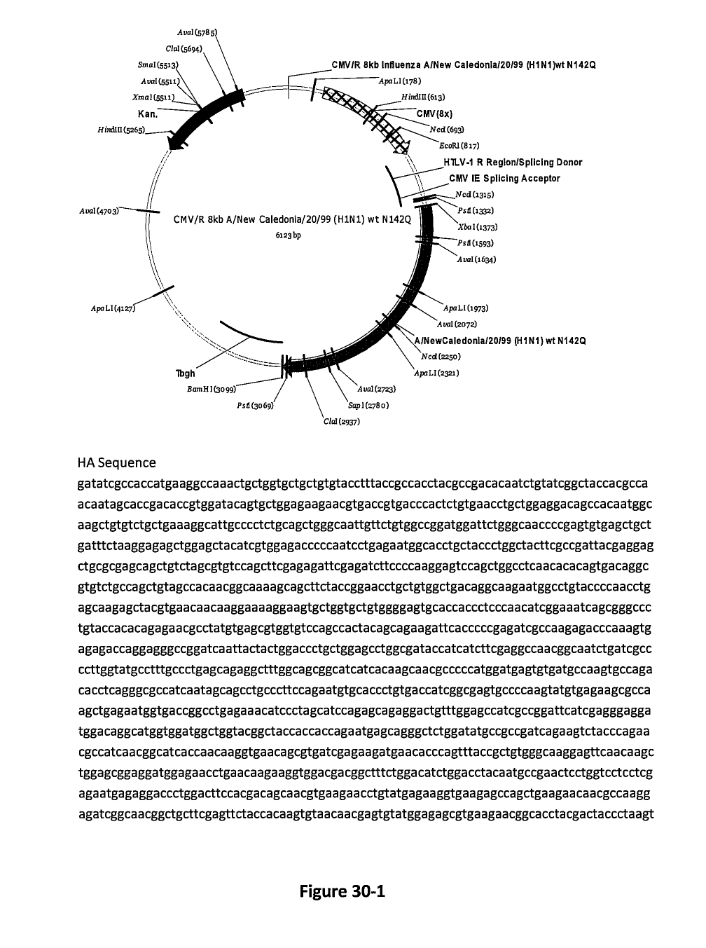

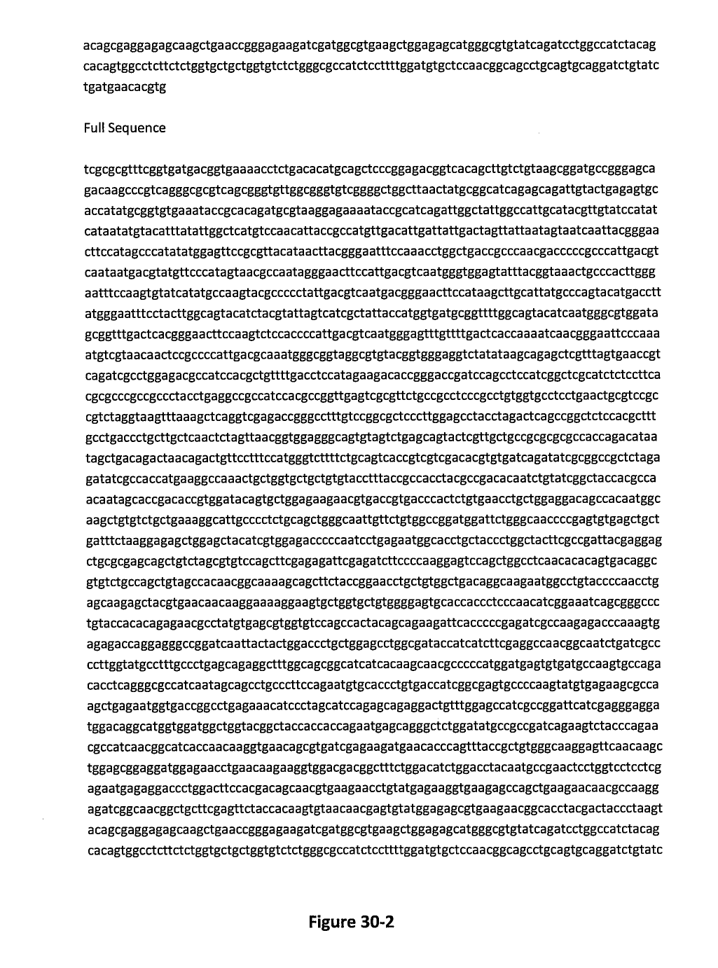

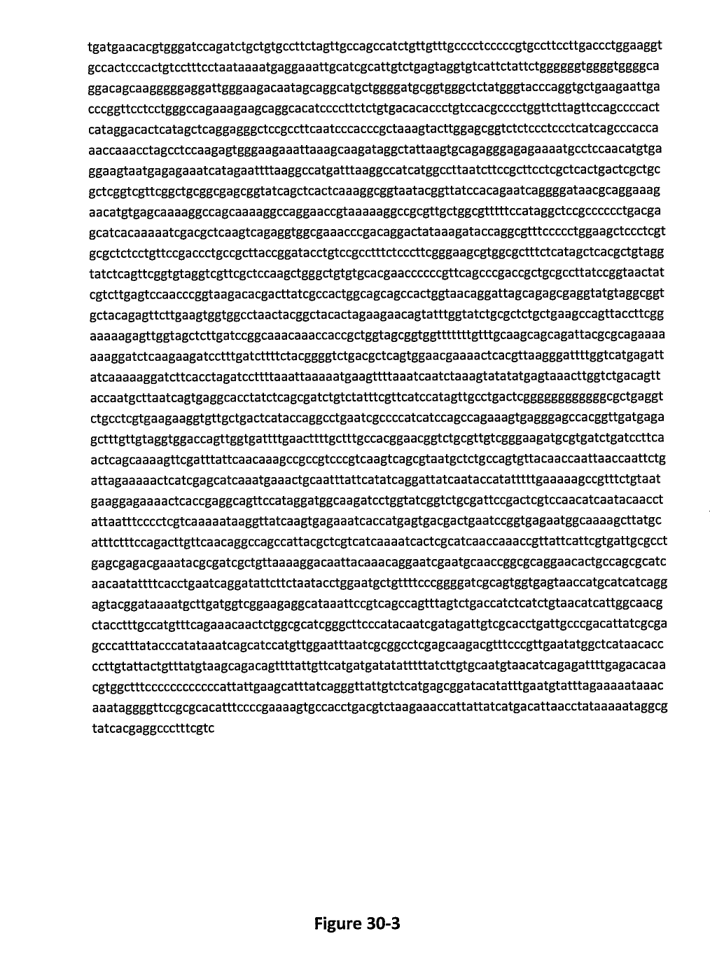

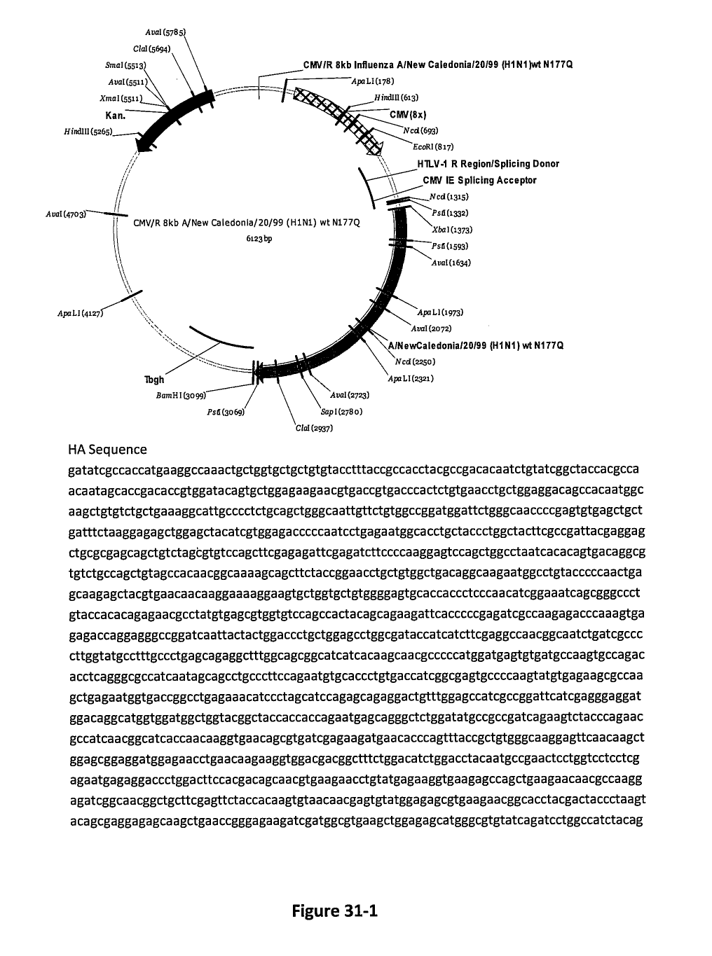

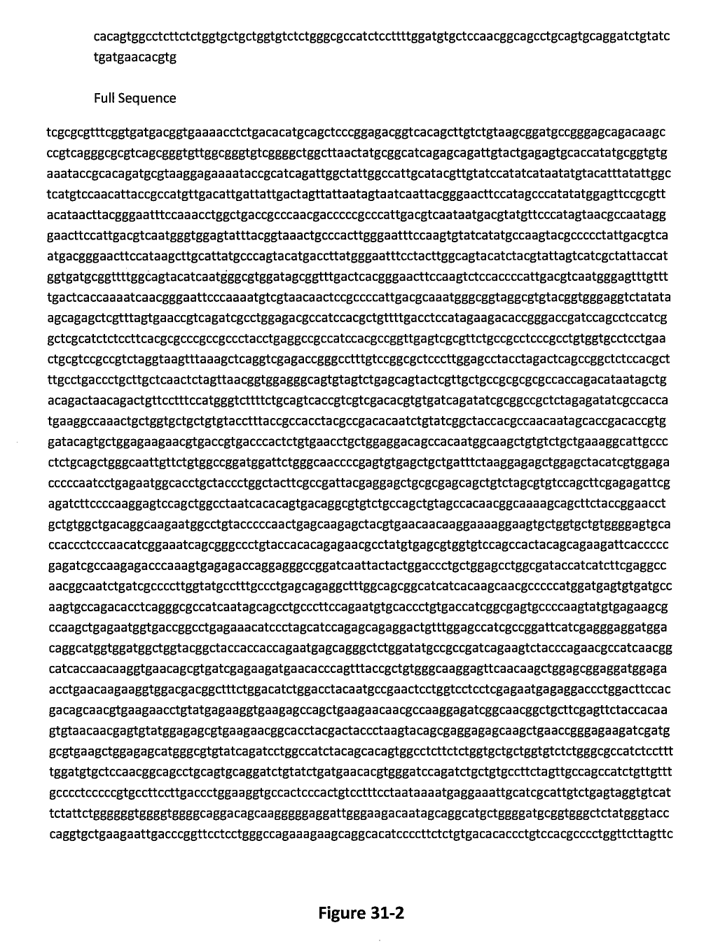

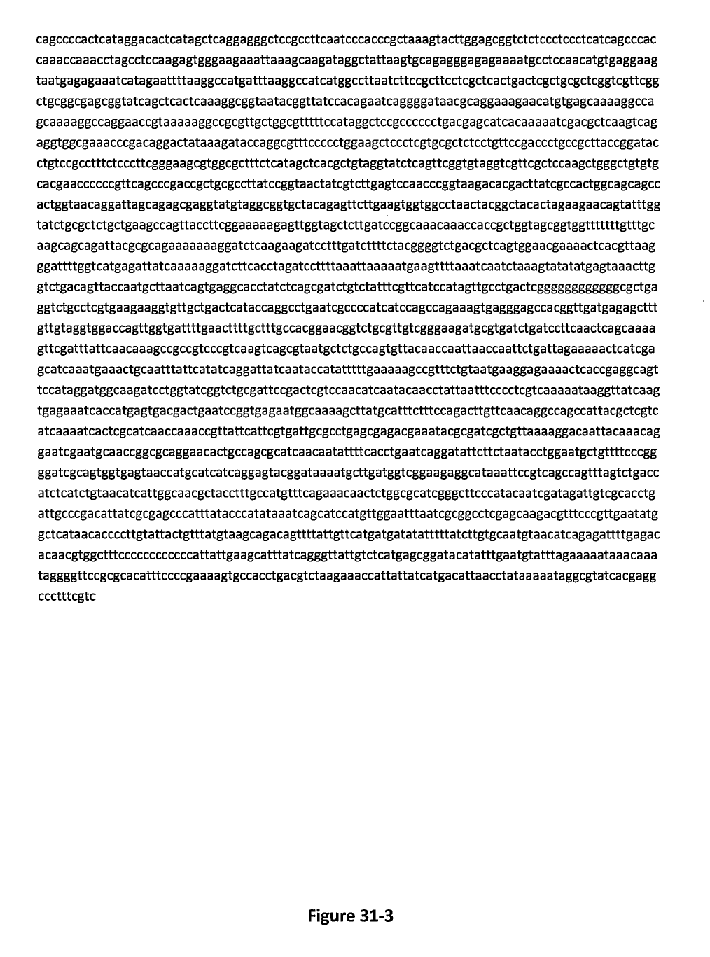

To examine whether there was cross-reactive neutralization between 1918 and 2009 H1N1 pandemic influenza viruses, mice were immunized with a nucleic acid construct encoding hemagglutinin (HA) from influenza A (H1N1) 2009 (CA 04/09) [VRC 9328; SEQ ID NO:63], A (H1N1) 1918 (SC) [VRC 7730; SEQ ID NO:1] or A (H1N1) 1999 (NC) [VRC 7722; FIG. 18], and the specificity of the neutralizing immune response assessed. The immunization protocol has been described by Kong, W.-P. et al. (Protective immunity to lethal challenge of the 1918 pandemic influenza virus by vaccination. Proc. Natl. Acad. Sci. USA 103, 15987-15991 (2006)). Briefly, 6-8 week old, female BALB/c mice (Jackson Laboratories) were immunized intramuscularly at weeks 0, 3, and 6 with 15 μg of the indicated nucleic acid construct in 100 μl of phosphate buffered saline (PBS) at pH 7.4. Blood was then collected 14 days after each immunization and the serum isolated and assessed using the pseudotyped lentiviral reporter assay described by Yang, Z.-Y. et al. (Immunization by avian H5 influenza hemagglutinin mutants with altered receptor binding specificity. Science 317, 825-828 (2007)). Briefly, HA NA-pseudotyped lentiviral vectors encoding luciferase were first titrated by serial dilution. Similar amounts of virus (p24˜6.25 ng/ml) were then incubated with indicated amounts of mouse antisera for 20 minutes at room temperature (RT) and added to 293A cells (10,000 cells per well; 50 μl/well, in triplicate). For 1918 SC-pseudotyped and 1999 NC-pseudotyped vectors, plates were washed and replaced with fresh media 2 hours later, and luciferase activity was measured after 24 hours. For 2009 CA-pseudotyped vectors, 293A cells were incubated with virus overnight and luciferase activity measured after 72 hours. Monoclonal antibody (mAb) C179 was used to standardize the input virus used in the neutralization assay. The results of this study are shown in FIG. 1a.

Remarkably, antisera from the H1N1 (1918 SC) immune mice neutralized heterologous A(H1N1) 2009 virus entry to a high titer, almost as high as the homologous strain (FIG. 1a, 1918, left vs. middle panel). Conversely, antisera from A (H1N1) 2009 immune mice neutralized both viruses to a high titer in contrast to non-immune sera or to antisera to a seasonal influenza virus, H1N1 1999 (New Caledonia) (FIG. 1a, 2009 vs. control and NC, left and middle panels). In contrast, the seasonal 1999 NC antisera showed strong homologous neutralization but failed to neutralize either the 2009 CA or the 1918 SC virus (FIG. 1a, 1999 NC, right vs. left and middle panels). These results were unexpected, given the longer chronologic separation of A (H1N1) 2009 (CA 04/09) from 1918 than 1999.

Similar cross-reactivity was observed using a hemagglutination inhibition (HI) assay previously described by Yang (Yang, Z.-Y. et al. Immunization by avian H5 influenza hemagglutinin mutants with altered receptor binding specificity. Science 317, 825-828 (2007)). The results of this experiment are shown in FIG. 1b. Vaccine sera directed to 1918 SC showed the highest HI titer to matched virus and recognized A (H1N1) 2009 but not seasonal 1999 NC (FIG. 1b, left panel). Similarly, A (H1N1) 2009 immune sera reacted with A (H1N1) 2009 and, to a lesser extent, 1918 SC but not 1999 NC (FIG. 1b, middle panel), while 1999 NC immune sera exhibited homologous HI reactivity only (FIG. 1b, right panel).

To understand the mechanism of cross-neutralization, competition studies were performed. Purified recombinant 1918 SC or A (H1N1) 2009 trimeric HA was used to block neutralization of 1918 SC, A (H1N1) 2009, or 1999 NC virus. The results of these studies are shown in FIG. 1c. While these trimeric HA proteins were able to inhibit neutralization by antisera to both pandemic viruses, they failed to inhibit the seasonal 1999 NC virus (FIG. 1c). Conversely, the 1999 NC trimeric HA inhibited autologous virus but did not block neutralization by either the 1918 SC or A (H1N1) 2009 (CA 04/09) antisera (FIG. 1c, far right panel). Recent studies have shown the presence of a highly conserved domain in the stem of the viral HA, a potential structure that might be involved in cross-neutralization of diverse viruses [Okuno, Y., Isegawa, Y., Sasao, F. & Ueda, S. A common neutralizing epitope conserved between the hemagglutinins of influenza A virus H1 and H2 strains. J. Virol. 67, 2552-2558 (1993); Ekiert, D. C. et al. Antibody recognition of a highly conserved influenza virus epitope. Science 324, 246-251 (2009); Sui, J. et al. Structural and functional bases for broad-spectrum neutralization of avian and human influenza A viruses. Nat. Struct. Mol. Biol. 16, 265-273 (2009)]; however, there is greater than 94% identity between the seasonal and pandemic viruses in this region. Because no inhibition was observed with the 1999 NC and this virus is sensitive to neutralization by anti-stem antibodies [Okuno, Y., Isegawa, Y., Sasao, F. & Ueda, S. A common neutralizing epitope conserved between the hemagglutinins of influenza A virus H1 and H2 strains. J. Virol. 67, 2552-2558 (1993); Smirnov, Y. A. et al. An epitope shared by the hemagglutinins of H1, H2, H5, and H6 subtypes of influenza A virus. Acta Virol. 43, 237-244 (1999)], these data suggested that the conserved stem region of the spike was not likely a target of neutralization. Instead, these cross-inhibition studies implicated the head region as the site of neutralization.

### Example 2

To determine whether the immune responses observed in Example 1 provided cross-protection in vivo, mice were immunized with inactivated virus vaccines derived from pandemic or seasonal influenza viruses and challenged with a highly lethal mouse-adapted A (H1N1) 2009 virus. Inactivated virus was prepared by concentrating virus from allantoic fluid, purifying the virus on a linear sucrose gradient, and then treating the purified virus at a concentration of 1 mg/ml with 0.025% formalin at 4C for 3 days. This treatment results in complete loss of infectivity of the virus. Groups of BALB/c mice (n=12) were anesthetized with Avertin (Sigma-Aldrich, St Louis, Mo.) and injected intramuscularly (i.m.) with 10 μg of formalin-inactivated vaccine. Mice received two inoculations at an interval of 3 weeks, and were challenged 6 weeks after the initial vaccination. For challenge, anesthetized mice received 50 μl of infectious A/California/04/2009 virus (10⁶ PFU) diluted in PBS and inoculated intranasally. Four days later, four mice from each group were euthanized and lungs (n=4) were collected and homogenized in 1 ml of cold PBS. Solid debris was pelleted by centrifugation and tissues were titrated for virus infectivity in a standard plaque assay. The eight remaining mice in each group were checked daily for disease signs and death for 21 days post-challenge. The results of this study are shown below in Tables 1A and 1B.

TABLE 1
Protective efficacy of H1N1 vaccines against pandemic
influenza A (H1N1) 2009 virus in mice.
A.

| | Weight | Mean virus | No. |
|---|---|---|---|
| | loss | titer in lung | protected/ |
| Vaccine group | (%) | (log₁₀PFU/ml) | total no |
| PBS | 18.5 | 6.8 | 0/8 |
| 1999 Pan (H3N2) | 18.8 | 6.1 | 0/8 |
| 1999 NC | 17.9 | 5.5 | 1/8 |
| 2007 Brisbane | 13 | 5.6 | 2/8 |
| 1918 SC | 3.7 | ≦0.9 | 8/8^{e} |
| 2009 (Mex/4108) | 1.7 | ≦0.9 | 8/8^{e} |

B.

| Vaccine group | HI antibody titer to homologous virus |
|---|---|
| PBS | <10 |
| 1999 Panama/2007/1999 (H3N2) | 76 |
| A/New Caldonia/20/1999 | 80 |
| A/Brisbane/59/2007 | 57 |
| A/SouthCarolina/1/1918 | 59 |
| A/Mexico/4108/2009 | 50 |

The results show that animals immunized with the 1918 SC or A (H1N1) 2009 inactivated virus vaccines were completely protected from lethality and showed >5 logs reduction in viral titers, in contrast to seasonal influenza vaccines or non-immune controls. Immunization with the 1918 SC pandemic strain vaccine therefore conferred protection against A (H1N1) 2009, documenting its ability to cross-protect in vivo. Conversely, immunization with A (H1N1) 2009 vaccine also protected mice from lethal 1918 SC challenge.

Immunization with a nucleic acid construct encoding A/California/04/2009 (H1N1) HA [VRC 9328; SEQ ID NO:63] also protected mice from challenge by 1918 SC or 2009 CA. For this study, groups of mice BALB/c mice (n=10) were anesthetized as described above, and injected intramuscularly (i.m.) with 15 μg of empty vector or a nucleic acid construct encoding the 2009 CA HA protein. Mice were inoculated at 0, 2 and 4 weeks, and were challenged 7 weeks after the initial innoculation. For challenge, anesthetized mice were intransally administered 50 μl of PBS of either 17,000 LD50 of mouse-adapted A/California/04/2009 virus or 100 LD50 A/South Carolina/1918 virus. Four days later, four mice from each group were euthanized and lungs were collected and homogenized in 1 ml of cold PBS. Solid debris was pelleted by centrifugation and tissues were titrated for virus infectivity in a standard plaque assay. The eight remaining mice in each group were checked daily for disease signs and death for 21 days post-challenge. The results of this study are shown below in Table 2.

TABLE 2
Protective efficacy of 2009 CA HA DNA vaccine
against 1918 SC and 2009 CA viruses in mice

| | | | Mean Virus | Number |
|---|---|---|---|---|
| | Chal- | Weight | Titer in Lung | protected/ |
| | lenge | Loss | (log₁₀ | total |
| Vaccine Group | Virus | (%) | PFU/ml) | number |
| Control DNA | 2009 CA | 18.0 ± 0.00 | 7.48 ± 0.00 | 0/5 |
| A/California/ | 2009 CA | 1.1 ± 2.19 | 5.44 ± 0.96 | 5/5 |
| 04/2009 | | | | |
| Control DNA | 1918 SC | 19.4 ± 5.34 | 6.46 ± 0.08 | 0/5 |
| A/California/ | 1918 SC | 5.9 ± 6.91 | 5.46 ± 0.19 | 5/5 |
| 04/2009 | | | | |

### Example 3

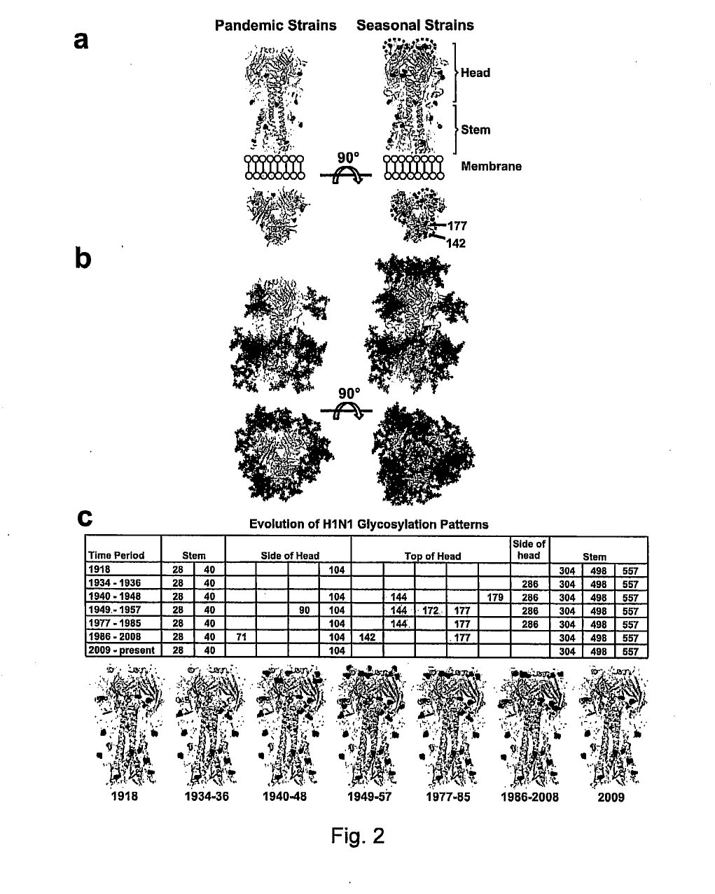

This Example describes studies aimed at further defining the molecular basis of cross-neutralization by examining the amino acid diversity and glycosylation site conservation among diverse HAs. The amino acid identity between the 1918 SC and A (H1N1) 2009 HA proteins within the globular head is approximately 79.8% (amino acids 64-286, 1918 numbering). This level of amino acid divergence was similar to the divergence among seasonal influenza viruses and would likely confer resistance to antibody neutralization; however, the top part of the RBD adjacent to the 2,6-sialic acid recognition sites includes a large region (amino acids 131-143, 170-182, 205-215 and 257-262, 1918 numbering) of over 6000 Å² per trimer that is 95% conserved between these pandemic strains. (Surface area was calculated using AREAIMOL in the CCP4 suite; Collaborative Computational Project, Number 4, The CCP4 Suite: Programs for protein crystallography. Acta Crystallogr. D. Biol. Crystallogr. 50, 760-763 (1994)). In contrast, there was a notable difference in conserved glycosylation sites on the RBD in pandemic and seasonal strains. The pandemic viruses from 1918 and 2009 lacked two glycosylation sites (142 and 177, 1918 numbering) on the head of the spike (FIGS. 2a and b, left panels). These sites are highly conserved among seasonal influenza viruses, exemplified by 1999 NC virus (FIGS. 2a and b, right panels). Modeling of these glycosylation sites reveals the extensive additional chemical structure conferred by carbohydrate modification that would serve to shield the receptor binding domain from antibody neutralization (FIG. 2b, right vs. left panel). The presence of these glycosylation sites was analyzed further using the NCBI Influenza Virus Resource (Bao, Y. et al. The influenza virus resource at the National Center for Biotechnology Information. J. Virol. 82, 596-601 (2008)). This analysis revealed the acquisition of two glycosylation sites on the top of the RBD by the early 1940s (FIG. 2c and Table 4). As can be seen in Table 3, from 1977 to 2008, the presence of at least one of the two glycosylation sites is observed in 97.8% of seasonal strains, and both glycosylation sites in 87.8%.

TABLE 3
The evolution of human H1N1 HA glycosylation patterns from 1918 to 2009

| Strain | Stem | | Side of head | | | | Top of head | | | | | Side of head | Stem | | |
|---|---|---|---|---|---|---|---|---|---|---|---|---|---|---|---|
| H1N1/South Carolina/1918 | 28 | 40 | | | | 104 | | | | | | | 304 | 498 | 557 |
| H1N1/WSN/1933a | 28 | | | 73 | | | 142 | | | | | 286 | | 498 | 557 |
| H1N1/WSN/1933b | 28 | | | 73 | | | 142 | | | | | 286 | | 498 | 557 |
| H1N1/WSN/TS61/1933 | 28 | | | 73 | | | 142 | | | | | 286 | | 498 | 557 |
| H1N1/Wilson-Smith/1933a | 28 | 40 | | | | | | | | | 179 | 286 | 304 | 498 | 557 |
| H1N1/Wilson-Smith/1933b | 28 | 40 | | 73 | | | 142 | | | | | 286 | | 498 | 557 |
| H1N1/PR/8 1934 | 28 | 40 | | | | | | 144 | | | | 286 | 304 | 498 | 557 |
| H1N1/Puerto Rico/8/1934a | 28 | 40 | | | | | | | | | | 286 | 304 | 498 | 557 |
| H1N1/Puerto Rico/8/1934b | 28 | 40 | | | | | | | | | | 286 | 304 | 498 | 557 |
| H1N1/Puerto Rico/8/1934c | 28 | 40 | | | | | | | | | | 286 | 304 | 498 | 557 |
| H1N1/Puerto Rico/8-SV14/1934 | 28 | 40 | | | | | | | | | | 286 | 304 | 498 | 557 |
| H1N1/Puerto Rico/8/Mt Sinai/1934 | 28 | 40 | | | | | | | | | | 286 | 304 | 498 | 557 |
| H1N1/Alaska/1935 | 28 | 40 | | | | | | | | | | 286 | 304 | 498 | 557 |
| H1N1/Philadelphia/1935 | 28 | 40 | | | | | | 144 | | | | 286 | 304 | 498 | 557 |
| H1N1/Melbourne/1935 | 28 | 40 | | | | | | 144 | | | | 286 | 304 | 498 | 557 |
| H1N1/Henry/1936 | 28 | 40 | | | | | | | | | | 286 | 304 | 498 | 557 |
| H1N1/Hickox/1940 | 28 | 40 | | | | 104 | | | | | 179 | 286 | 304 | 498 | 557 |
| H1N1/Bellamy/1942 | 28 | 40 | | | | 104 | | 144 | | | 179 | 286 | 304 | 498 | 557 |
| H1N1/Weiss/1943 | 28 | 40 | | | | 104 | | | | | | 286 | 304 | 498 | 557 |
| H1N1/Iowa/1943 | 28 | 40 | | | | 104 | | | | | 178 | 286 | 304 | 498 | 557 |
| H1N1/Marton/1943 | 28 | 40 | | | | 104 | | 144 | | | 179 | 286 | 304 | 498 | 557 |
| H1N1/Huston/1945 | 28 | 40 | | | | 104 | | 144 | | | 179 | 286 | 304 | 498 | 557 |
| H1N1/Cameron/1946 | 28 | 40 | | | | | | | | | 179 | 286 | 304 | 498 | 557 |
| H1N1/1947 FM/1/1947a | 28 | 40 | | | | | | 144 | | | | 286 | 304 | 498 | 557 |
| H1N1/1947 FM/1/1947b | 28 | 40 | | | | | | 144 | | | | 286 | 304 | 498 | 557 |
| H1N1/Albany/4835/1948 | 28 | 40 | | | | 104 | | 144 | | | 179 | 286 | 304 | 498 | 557 |
| H1N1/Roma/1949 | 28 | 40 | | | | 104 | | 144 | | 177 | | 286 | 304 | 498 | 557 |
| H1N1/Albany/4836/1950 | 28 | 40 | | | | 104 | | 144 | | | | 286 | 304 | 498 | 557 |
| H1N1/Fort Worth/1950 | 28 | 40 | | | 90 | | | | 172 | | | | 304 | 498 | 557 |
| H1N1/Albany/12/1951 | 28 | 40 | | | 90 | 104 | | 144 | 172 | 177 | | 286 | 304 | 498 | 557 |
| H1N1/Albany/14/1951 | 28 | 40 | | | 90 | 104 | | 144 | 172 | 177 | | 286 | 304 | 498 | 557 |
| H1N1/Albany/1618/1951 | 28 | 40 | | | 90 | 104 | | 144 | 172 | 177 | | 286 | 304 | 498 | 557 |
| H1N1/Leningrad/1954 | 28 | 40 | | | | 104 | | 144 | | 177 | | 286 | 304 | 498 | 557 |
| H1N1/Malaysia/1954 | 28 | 40 | | | 90 | | | 144 | | | | 286 | 304 | 498 | 557 |
| H1N1/Denver/1957 | 28 | 40 | | | | 104 | 142 | | 172 | 177 | | | 304 | 498 | 557 |
| H1N1/Tokyo/3/1967 | 28 | 40 | | | | | | | | | | 286 | 304 | 498 | 557 |
| H1N1/New Jersey/1976 | 28 | 40 | | | | 104 | | | | | | | 304 | 498 | 557 |
| H1N1/NewJersey/8/1976 | 28 | 40 | | | | 104 | | | | | | | 304 | 498 | 557 |
| H1N1/New Jersey/11/1976 | 28 | 40 | | | | 104 | | | | | | | 304 | 498 | 557 |
| H1N1/Tientsin/1977 | 28 | 40 | | | | 104 | | 144 | | 177 | | 286 | 304 | 498 | 557 |
| H1N1/USSR90/1977a | 28 | 40 | | | | 104 | | 144 | | 177 | | 286 | 304 | 498 | 557 |
| H1N1/USSR90/1977b | 28 | 40 | | | | 104 | | 144 | | 177 | | 286 | 304 | 498 | 557 |
| H1N1/USSR90/1977c | 28 | 40 | | | | 104 | | 144 | | 177 | | 286 | 304 | 498 | 557 |
| H1N1/USSR92/1977 | 28 | 40 | | | | 104 | | 144 | | 177 | | 286 | 304 | 498 | 557 |
| H1N1/Hong Kong/117/1977 | 28 | 40 | | | | 104 | | 144 | | 177 | | 286 | 304 | 498 | 557 |
| H1N1/Albany/20/1978 | 28 | 40 | | | | 104 | | 144 | | 177 | | 286 | 304 | 498 | 557 |
| H1N1/Arizona/14/1978 | 28 | 40 | | | | 104 | | 144 | | 177 | | 286 | 304 | 498 | 557 |
| H1N1/Brazil/11/1978 | 28 | 40 | | | | 104 | | 144 | | 177 | | 286 | 304 | 498 | 557 |
| H1N1/California/10/1978 | 28 | 40 | | | | 104 | | 144 | | 177 | | 286 | 304 | 498 | 557 |
| H1N1/California/45/1978 | 28 | 40 | | | | 104 | | 144 | | 177 | | 286 | 304 | 498 | 557 |
| H1N1/Lackland/03/1978 | 28 | 40 | | | | 104 | | 144 | | 177 | | 286 | 304 | 498 | 557 |
| H1N1/Lackland/07/1978 | 28 | 40 | | | | 104 | | | | 177 | | 286 | 304 | 498 | 557 |
| H1N1/Memphis/10/1978 | 28 | 40 | | | | 104 | | 144 | | 177 | | 286 | 304 | 498 | 557 |
| H1N1/Memphis/13/1978 | 28 | 40 | | | | 104 | | 144 | | 177 | | 286 | 304 | 498 | 557 |
| H1N1/USSR46/1979 | 28 | 40 | | | | 104 | | 144 | | 177 | | 286 | 304 | 498 | 557 |
| H1N1/India/6263/1980 | 28 | 40 | | | | 104 | | 144 | | 177 | | 286 | 304 | 498 | 557 |
| H1N1/Baylor/11515/1982 | 28 | 40 | | | | 104 | | | | 177 | | 286 | 304 | 498 | 557 |
| H1N1/Fiji/15899/1983 | 28 | 40 | | | | 104 | | 144 | | 177 | | 286 | 304 | 498 | 557 |
| H1N1/Memphis/1983 | 28 | 40 | | | | 104 | | 144 | | 177 | | 286 | 304 | 498 | 557 |
| H1N1/Tonga/14/1984 | 28 | 40 | | | | 104 | | 144 | | 177 | | 286 | 304 | 498 | 557 |
| H1N1/Mongolia/231/1985 | 28 | 40 | | | | 104 | | 144 | | 177 | | 286 | 304 | 498 | 557 |
| H1N1/Memphis/12/1986 | 28 | 40 | 71 | | | 104 | 142 | | | 177 | | 286 | 304 | 498 | 557 |
| H1N1/Singapore/6/1986 | 28 | 40 | 71 | | | 104 | 142 | | | 177 | | 286 | 304 | 498 | 557 |
| H1N1/Memphis/01/1987 | 28 | 40 | 71 | | | 104 | 142 | | | 177 | | 286 | 304 | 498 | 557 |
| H1N1/Mongolia/153/1988 | 28 | 40 | | | | | | | | | | 286 | 304 | 498 | 557 |
| H1N1/Suita/01/1989 | 28 | 40 | 71 | | | 104 | 142 | | | 177 | | 286 | 304 | 498 | 557 |
| H1N1/Texas/36/1991 | 28 | 40 | 71 | | | 104 | 142 | | | 177 | | 286 | 304 | 498 | 557 |
| H1N1/Mongolia/162/1991 | 28 | 40 | 71 | | | 104 | 142 | | | 177 | | 286 | 304 | 494 | 553 |
| H1N1/Mongolia/111/1991 | 28 | 40 | | | | | | | | | | 286 | 304 | 493 | 552 |
| H1N1/New York/604/1995 | 28 | 40 | 71 | | | 104 | 142 | | | 177 | | | 304 | 498 | 557 |
| H1N1/Switzerland/5389/1995 | 28 | 40 | 71 | | | 104 | 142 | | | 177 | | 286 | 304 | 498 | 557 |
| H1N1/Beijing/262/1995 | 28 | 40 | 71 | | | 104 | | | | 177 | | | 304 | 498 | 557 |
| H1N1/New York/626/1996 | 28 | 40 | 71 | | | 104 | 142 | | | 177 | | | 304 | 498 | 557 |
| H1N1/TW/3355/1997 | 28 | 40 | 71 | | | 104 | 142 | | | 177 | | 286 | 304 | 498 | 557 |
| H1N1/Hong Kong/1131/1998 | 28 | 40 | 71 | | | 104 | 142 | | | 177 | | | 304 | 498 | 557 |
| H1N1/South Wales/18/1999 | 28 | 40 | 71 | | | 104 | 142 | | | 177 | | | 304 | 498 | 557 |
| H1N1/TW/4845/1999 | 28 | | 71 | | | 104 | 142 | | | 177 | | 286 | | 498 | 557 |
| H1N1/New Caledonia/20/1999 | 28 | 40 | 71 | | | 104 | 142 | | | 177 | | | 304 | 498 | 557 |
| H1N1/Canterbury/05/2000 | 28 | 40 | 71 | | | 104 | 142 | | | 177 | | | 304 | 498 | 557 |
| H1N1/Auckland/579/2000 | 28 | 40 | 71 | | | 104 | 142 | | | 177 | | | 304 | 498 | 557 |
| H1N1/Memphis/01/2001 | 28 | 40 | 71 | | | 104 | 142 | | | 177 | | | 304 | 498 | 557 |
| H1N1/New York/494/2002 | 28 | 40 | 71 | | | 104 | 142 | | | 177 | | | 304 | 498 | 557 |
| H1N1/Memphis/05/2003 | 28 | 40 | 71 | | | 104 | 142 | | | 177 | | | 304 | 498 | 557 |
| H1N1/Canterbury/106/2004 | 28 | 40 | 71 | | | 104 | 142 | | | 177 | | | 304 | 498 | 557 |
| H1N1/Auckland/619/2005 | 28 | 40 | 71 | | | 104 | 142 | | | 177 | | | 304 | 498 | 557 |
| H1N1/Thailand/CU41/2006 | 28 | 40 | 71 | | | 104 | 142 | | | 177 | | | 304 | 498 | 557 |
| H1N1/Solomon Islands/3/2006 | 28 | 40 | 71 | | | 104 | 142 | | | 177 | | | 304 | 498 | 557 |
| H1N1/Arizona/13/2007 | 28 | 40 | 71 | | | 104 | 142 | | | 177 | | | 304 | 498 | 557 |
| H1N1/Brisbane/59/2007 | 28 | 40 | 71 | | | 104 | 142 | | | 177 | | | 304 | 498 | 557 |
| H1N1/California/39/2007 | 28 | 40 | 71 | | | 104 | 142 | | | 177 | | | 304 | 498 | 557 |
| H1N1/Colorado/39/2007 | 28 | 40 | 71 | | | 104 | 142 | | | | | | 304 | 498 | 557 |
| H1N1/Florida/13/2007 | 28 | 40 | 71 | | | 104 | 142 | | | 177 | | | 304 | 498 | 557 |
| H1N1/Hawaii/46/2007 | 28 | 40 | 71 | | | 104 | 142 | | | 177 | | | 304 | 498 | 557 |
| H1N1/Illinois/11/2007 | 28 | 40 | 71 | | | 104 | 142 | | | | | | 304 | 498 | 557 |
| H1N1/Nebraska/02/2007 | 28 | 40 | 71 | | | 104 | 142 | | | 177 | | | 304 | 498 | 557 |
| H1N1/New Jersey/16/2007 | 28 | 40 | 71 | | | 104 | | | | 177 | | | 304 | 498 | 557 |
| H1N1/New York/16/2007 | 28 | 40 | 71 | | | 104 | 142 | | | 177 | | | 304 | 498 | 557 |
| H1N1/Oregon/07/2007 | 28 | 40 | 71 | | | 104 | 142 | | | 177 | | | 304 | 498 | 557 |
| H1N1/Texas/70/2007 | 28 | 40 | 71 | | | 104 | 142 | | | 177 | | | 304 | 498 | 557 |
| H1N1/Alaska/03/2008 | 28 | 40 | 71 | | | 104 | 142 | | | 177 | | | 304 | 498 | 557 |
| H1N1/California/07/2008 | 28 | 40 | 71 | | | 104 | 142 | | | 177 | | | 304 | 498 | 557 |
| H1N1/Denmark/05/2008 | 28 | 40 | 71 | | | 104 | 142 | | | 177 | | | 304 | 498 | 557 |
| H1N1/Georgia/05/2008 | 28 | 40 | 71 | | | 104 | 142 | | | 177 | | | 304 | 498 | 557 |
| H1N1/Idaho/09/2008 | 28 | 40 | 71 | | | 104 | 142 | | | | | | 304 | 498 | 557 |
| H1N1/Japan/AF07/2008 | 28 | 40 | 71 | | | 104 | 142 | | | 177 | | | 304 | 498 | 557 |
| H1N1/Massachusetts/01/2008 | 28 | 40 | 71 | | | 104 | 142 | | | 177 | | | 304 | 498 | 557 |
| H1N1/Minnesota/24/2008 | 28 | 40 | 71 | | | 104 | | | | 177 | | | 304 | 498 | 557 |
| H1N1/Missouri/06/2008 | 28 | 40 | 71 | | | 104 | | | | 177 | | | 304 | 498 | 557 |
| H1N1/Norway/256/2008 | 28 | 40 | 71 | | | 104 | 142 | | | 177 | | | 304 | 498 | 557 |
| H1N1/Stockholm/06/2008 | 28 | 40 | 71 | | | 104 | 142 | | | 177 | | | 304 | 498 | 557 |
| H1N1/Taiwan/70013/2008 | 28 | 40 | 71 | | | 104 | 142 | | | 177 | | | 304 | 498 | 557 |
| H1N1/California/04/2009 | 28 | 40 | | | | 104 | | | | | | | 304 | 498 | 557 |
| H1N1/Ancona/05/2009 | 28 | 40 | | | | 104 | | | | | | | 304 | 498 | 557 |
| H1N1/Canada-MB/RV2013/2009 | 28 | 40 | | | | 104 | | | | | | | 304 | 498 | 557 |
| H1N1/Japan/1070/2009 | 28 | 40 | | | | 104 | | | | | | | 304 | 498 | 557 |
| H1N1/Mexico/InDRE4114/2009 | 28 | 40 | | | | 104 | | | | | | | 304 | 498 | 557 |
| H1N1/Nanjing/1/2009 | 28 | 40 | | | | 104 | | | | | | | 304 | 498 | 557 |
| H1N1/New York/19/2009 | 28 | 40 | | | | 104 | | | | | | | 304 | 498 | 557 |
| H1N1/Paris/2722/2009 | 28 | 40 | | | | 104 | | | | | | | 304 | 498 | 557 |
| H1N1/Perth/29/2009 | 28 | 40 | | | | 104 | | | | | | | 304 | 498 | 557 |
| H1N1/Sao Paulo/43812/2009 | 28 | 40 | | | | 104 | | | | | | | 304 | 498 | 557 |
| H1N1/Stockholm/31/2009 | 28 | 40 | | | | 104 | | | | | | | 304 | 498 | 557 |
| H1N1/Texas/05/2009 | 28 | 40 | | | | 104 | | | | | | | 304 | 498 | 557 |

A few exceptions which lack these RBD glycosylation sites include the swine-related flu strains detected in 1976 as well as limited outbreaks detected in 1967, 1988 and 1991 (Table 3). These findings suggest that glycosylation on the top of the RBD is absent from pandemic viruses but present on nearly all seasonal influenza viruses, and the glycosylation sites of the RBD are likely to play a role in evading the human immune response.

TABLE 4
Evolution of H1N1 glycosylation and sequence variation in various time periods

| | | | | | | | | | | | | | | | | RBD-A |
|---|---|---|---|---|---|---|---|---|---|---|---|---|---|---|---|---|
| | | | | | | | | | | | | | | | | Conservation |
| | Stem | | Side of Head | | | | | Top of Head | | | | Side of Head | Stem | | | (%) |
| 1918 | 28 | 40 | | | | 104 | | | | | | | 304 | 498 | 557 | 100 |
| 1933 | 28 | | | 73 | | | 142 | | | | 179 | 286 | 304 | 498 | 557 | 80.5 |
| 1934-1939 | 28 | 40 | | | | | | 144 | | | | 286 | 304 | 498 | 557 | 73.9 |
| 1940-1948 | 28 | 40 | | | | 104 | | 144 | | | 179 | 286 | 304 | 498 | 557 | 66.1 |
| 1949-1957 | 28 | 40 | | | 90 | 104 | | 144 | 172 | 177 | | 286 | 304 | 498 | 557 | 63.2 |
| 1977-1985 | 28 | 40 | | | | 104 | | 144 | | 177 | | 286 | 304 | 498 | 557 | 65 |
| 1986-2008 | 28 | 40 | 71 | | | 104 | 142 | | | 177 | | 286 | 304 | 498 | 557 | 67.1 |
| 2009-Present | 28 | 40 | | | | 104 | | | | | | | 304 | 498 | 557 | 97.2 |

### Example 4

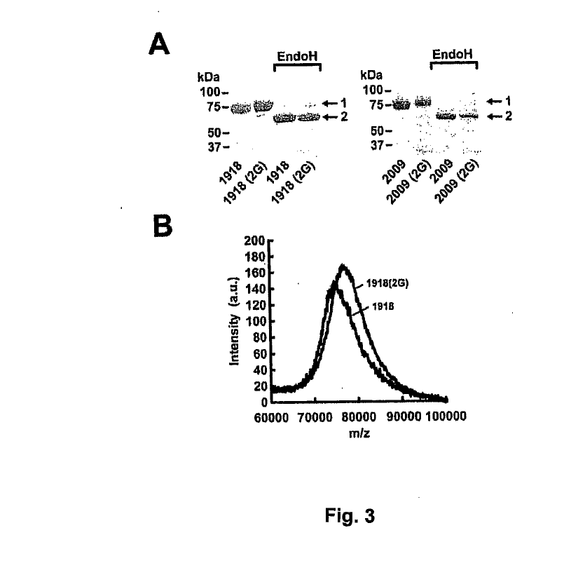

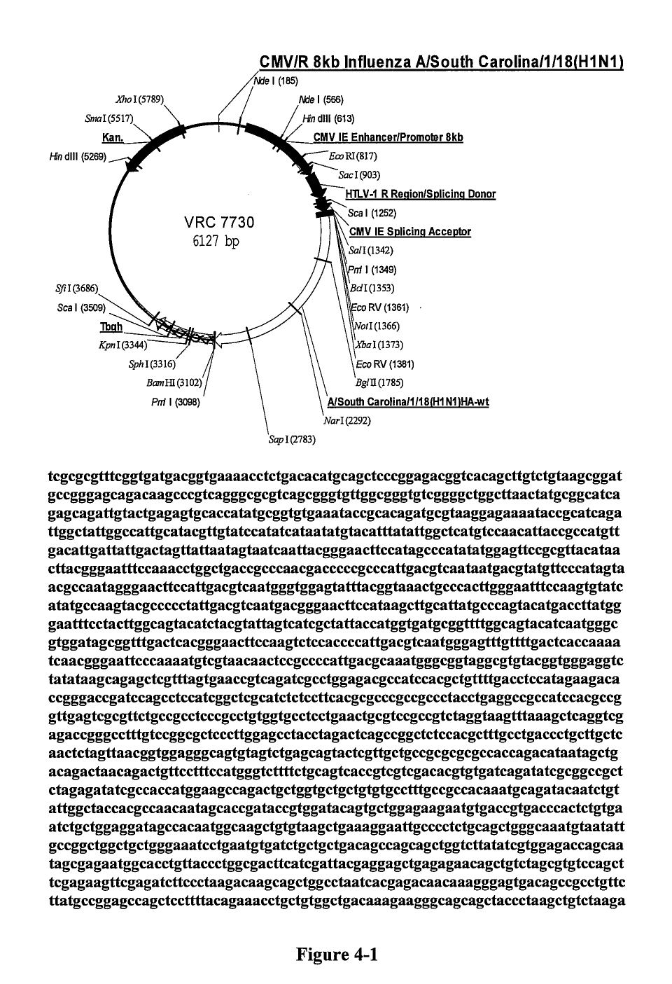

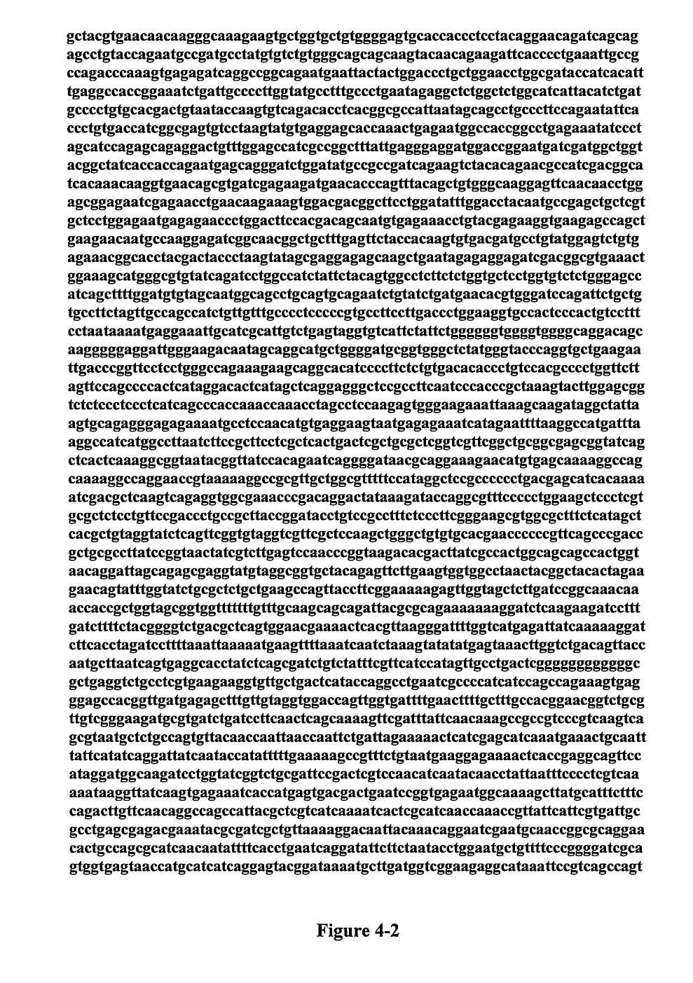

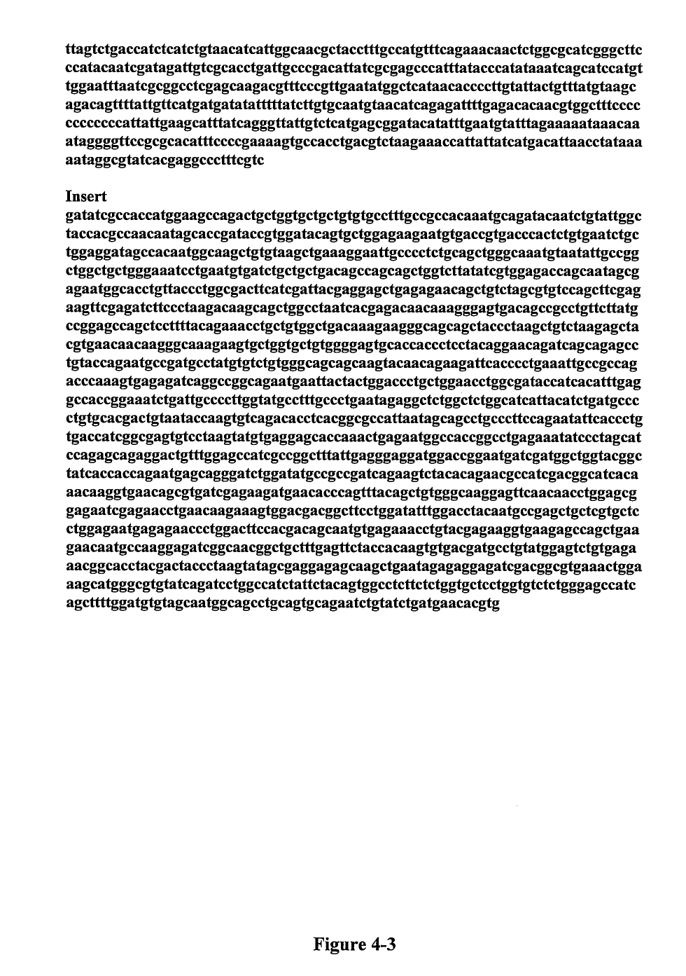

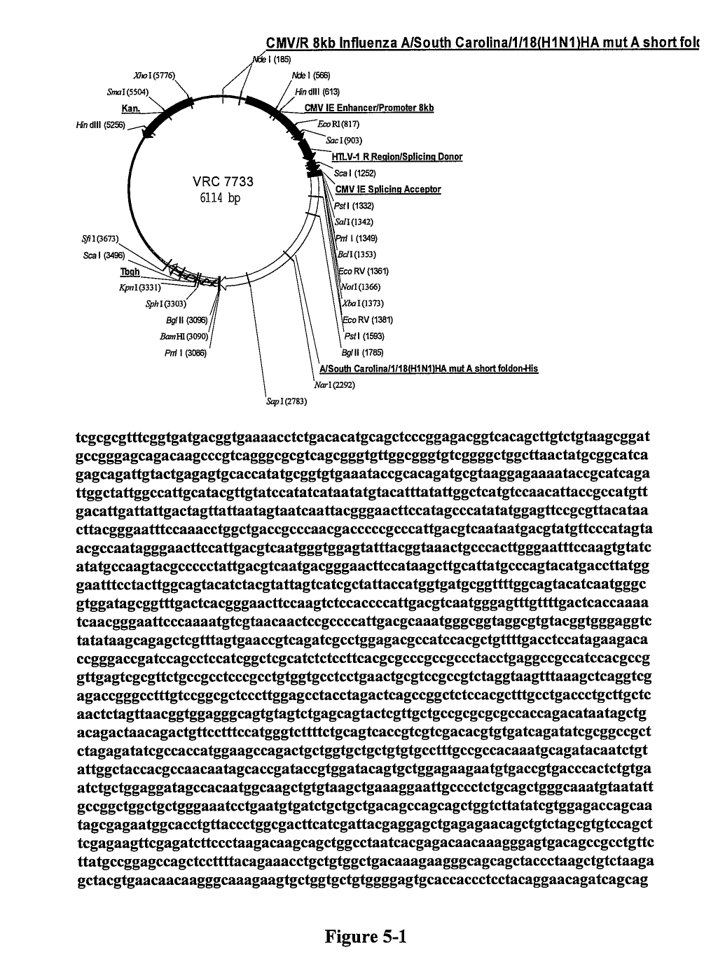

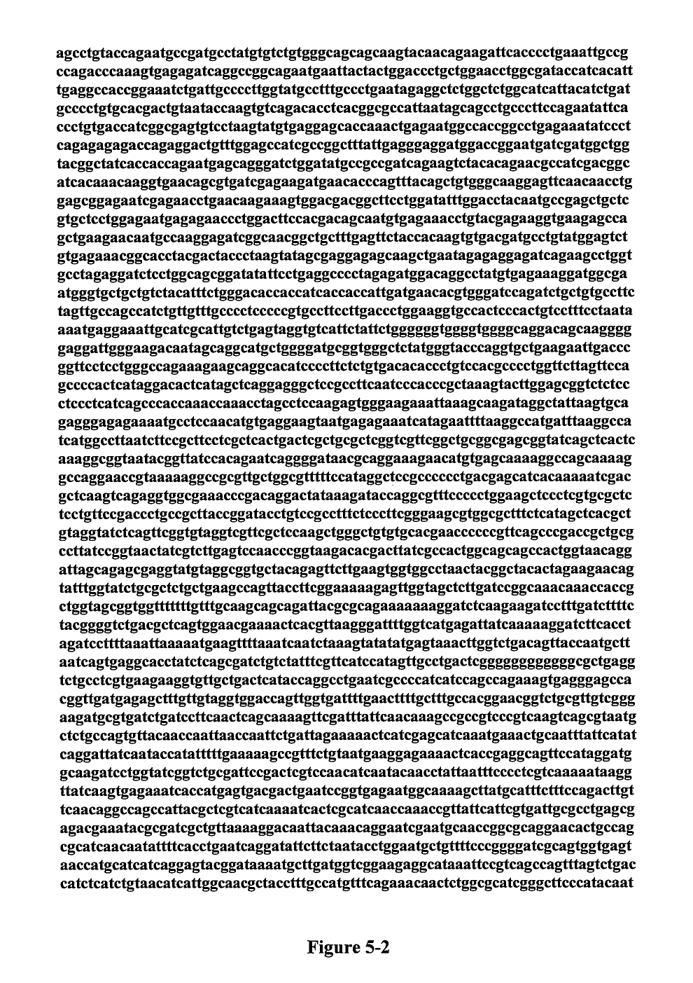

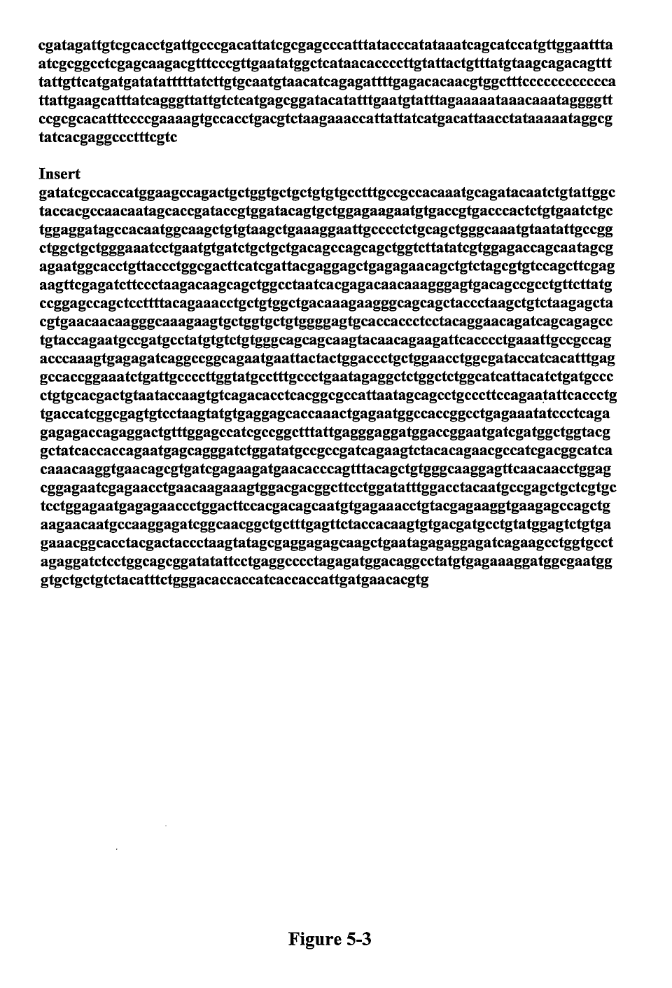

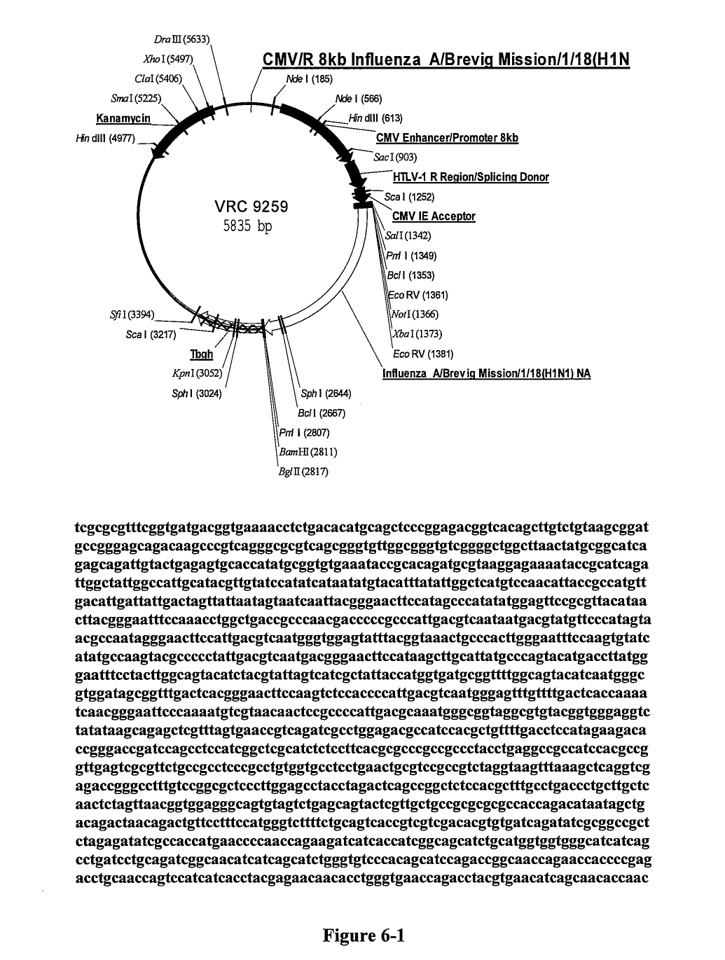

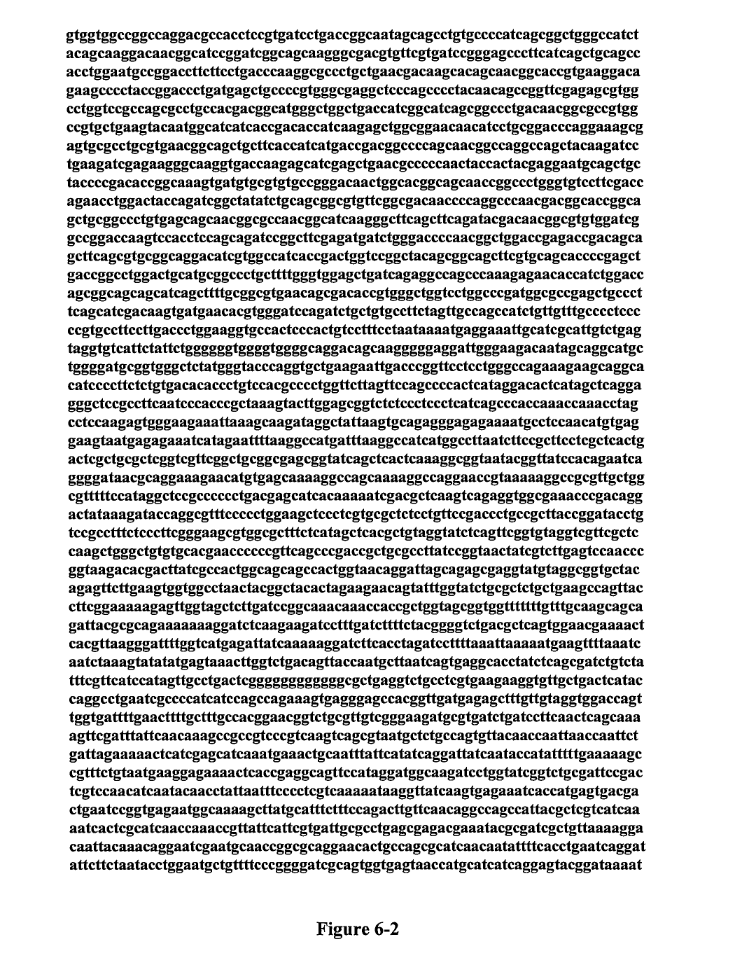

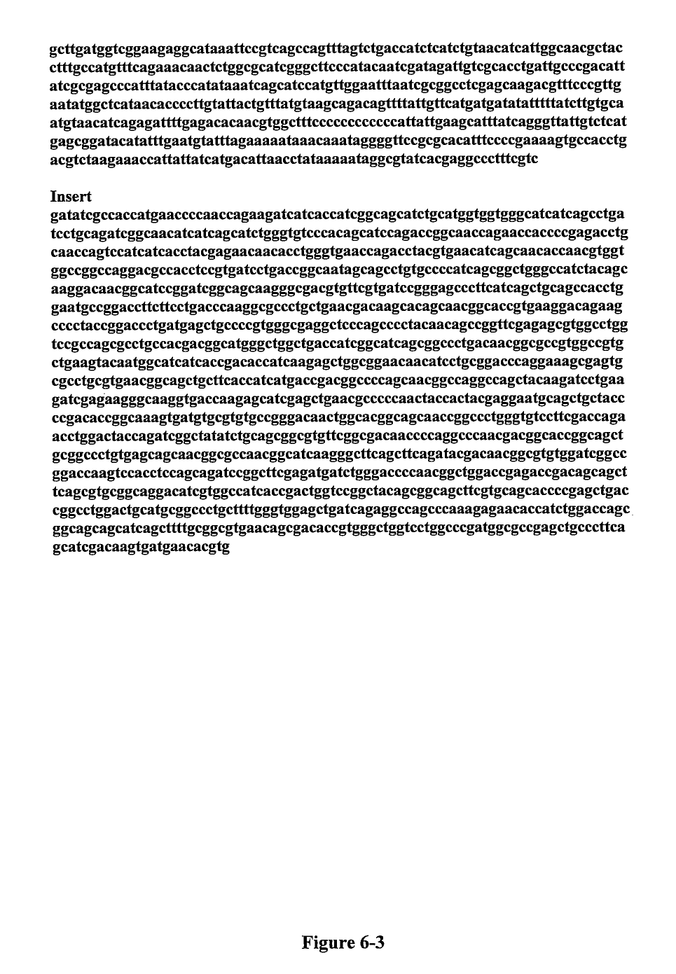

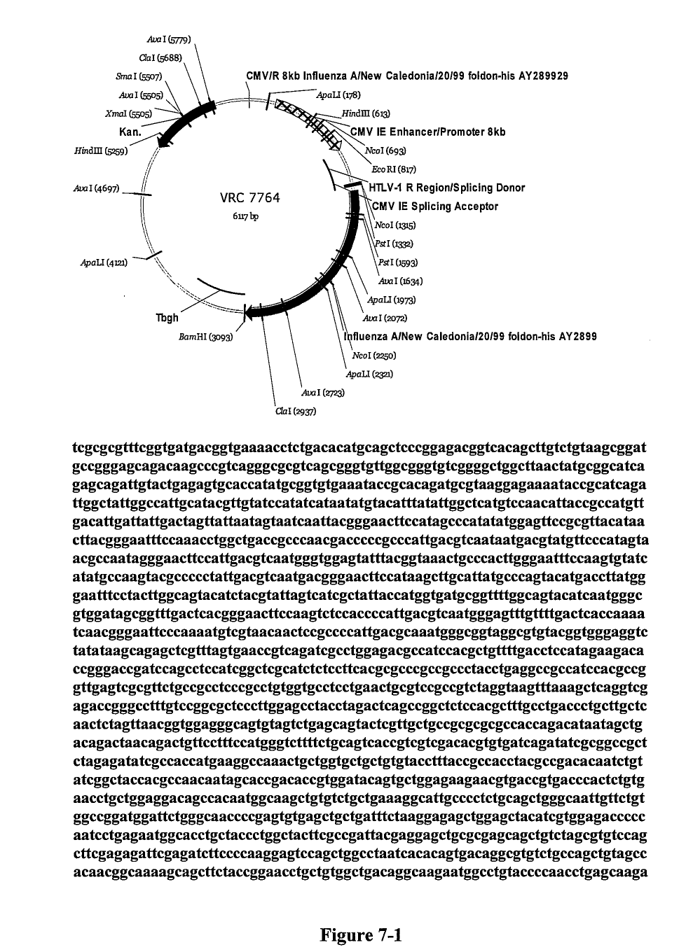

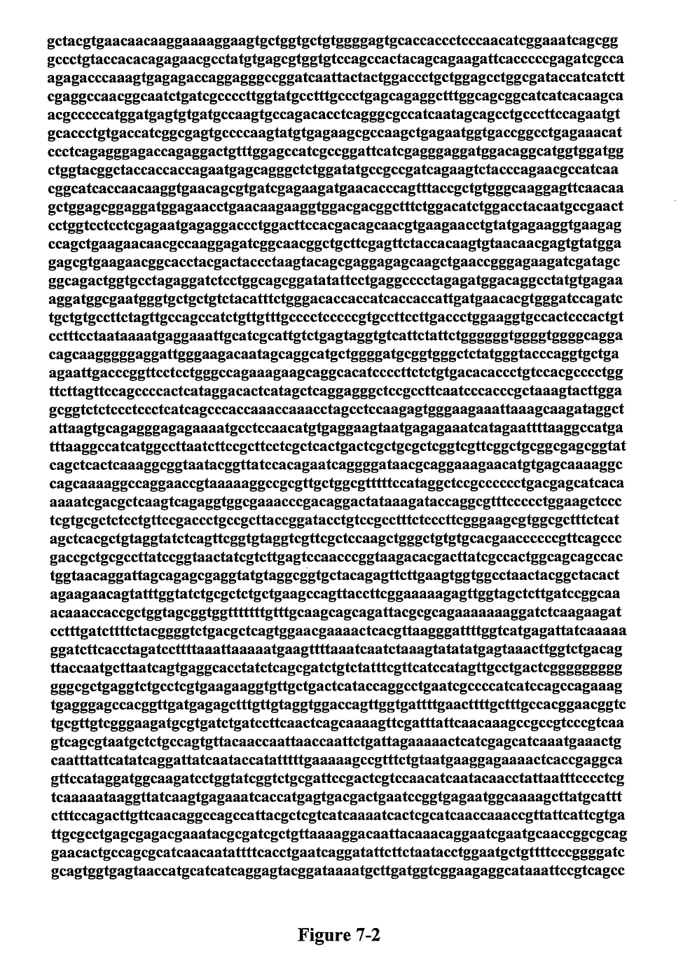

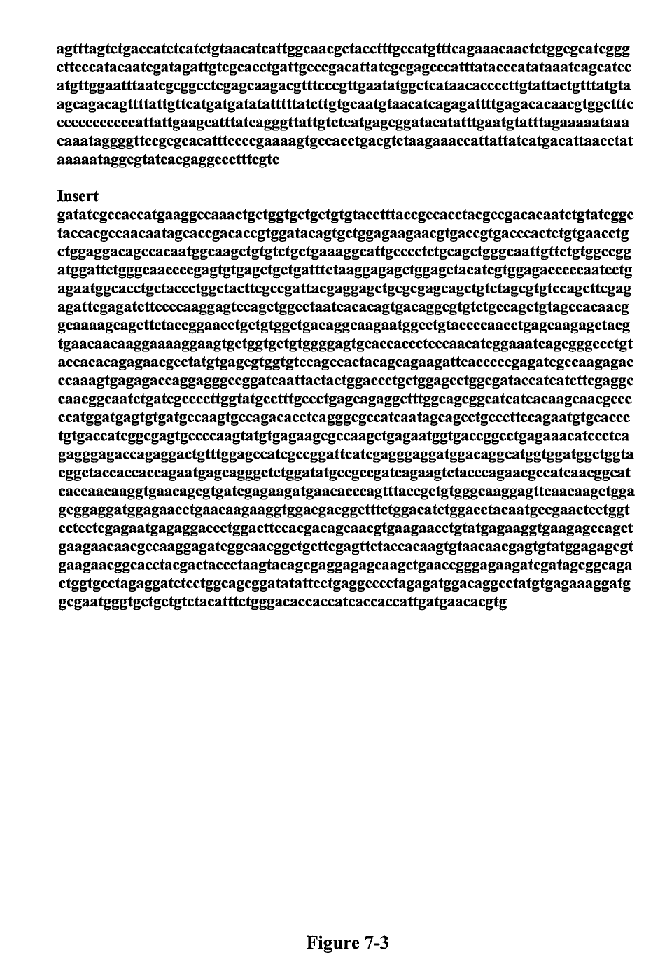

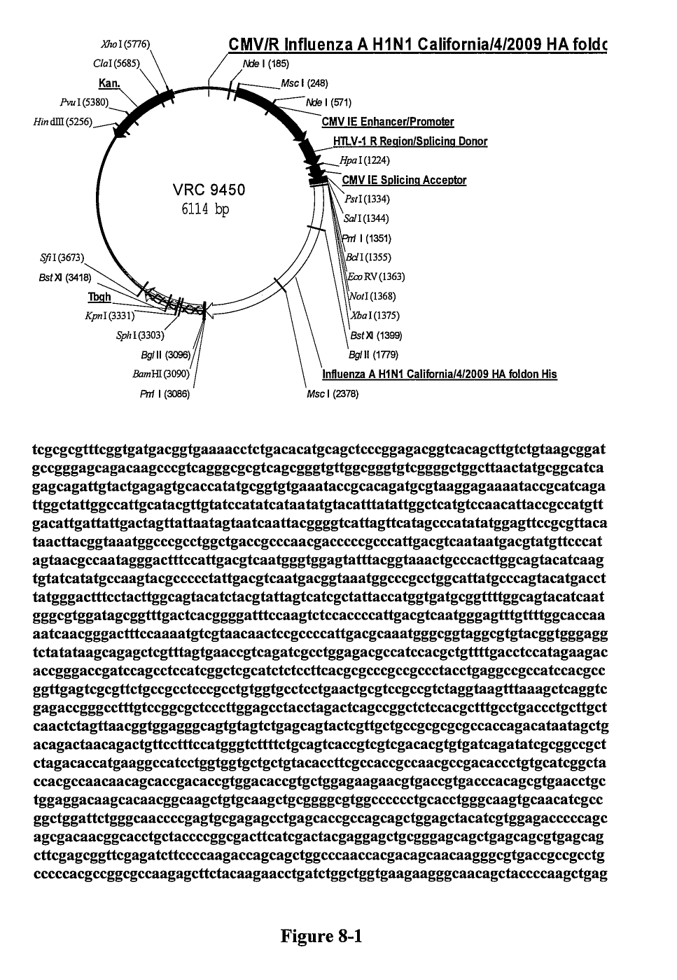

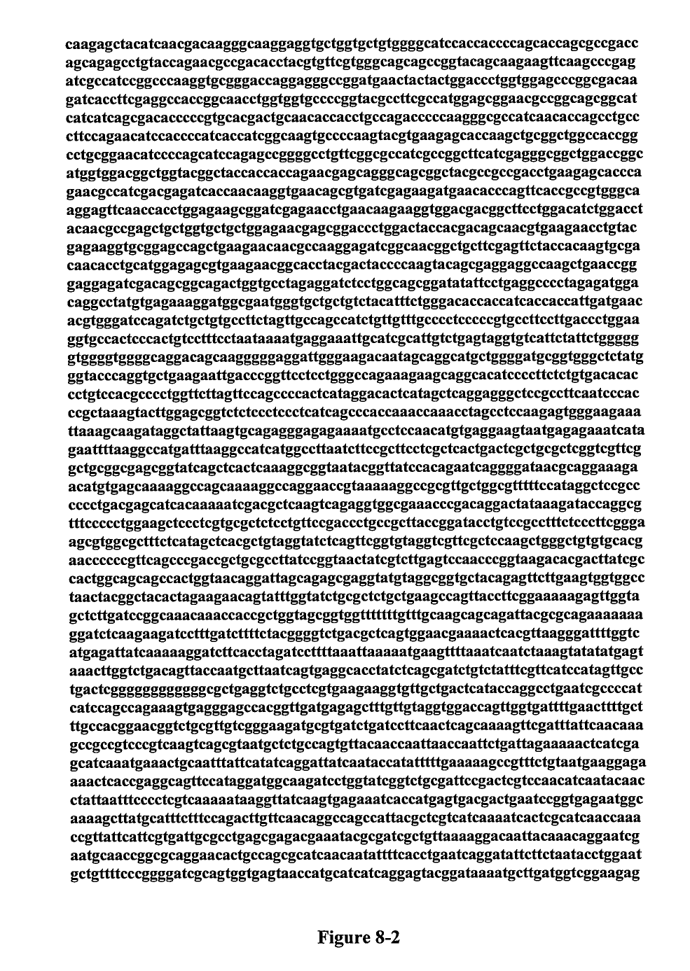

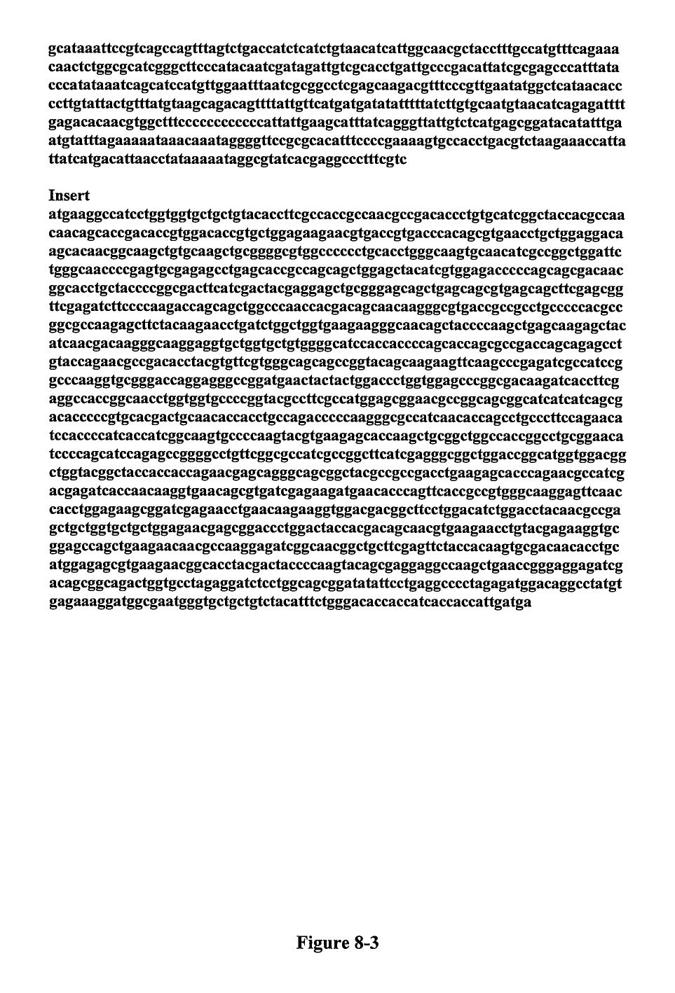

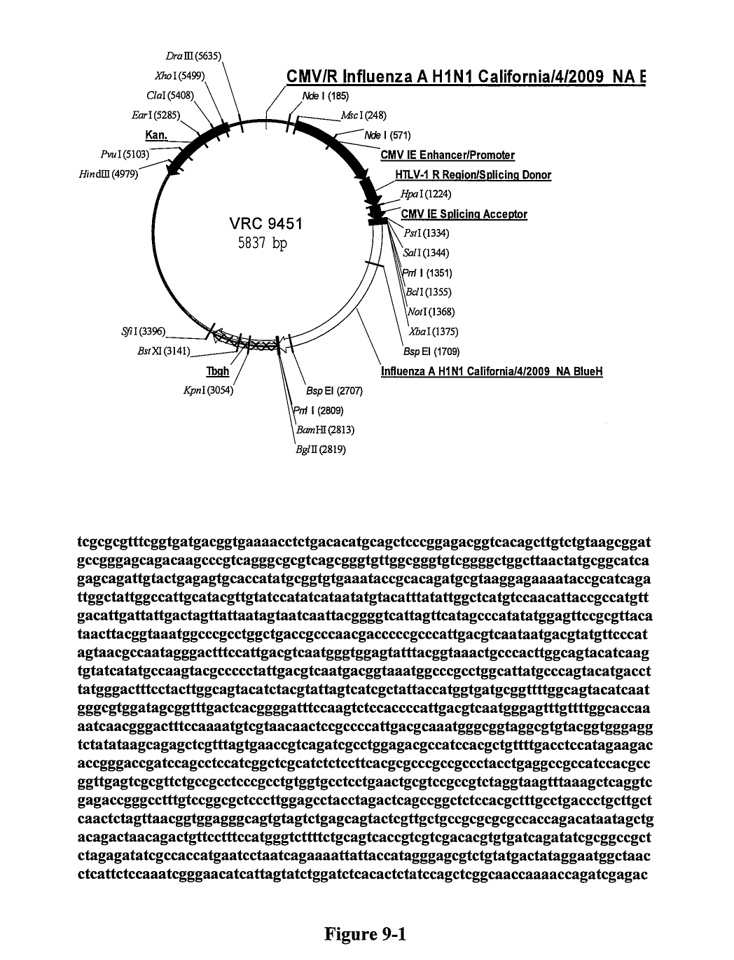

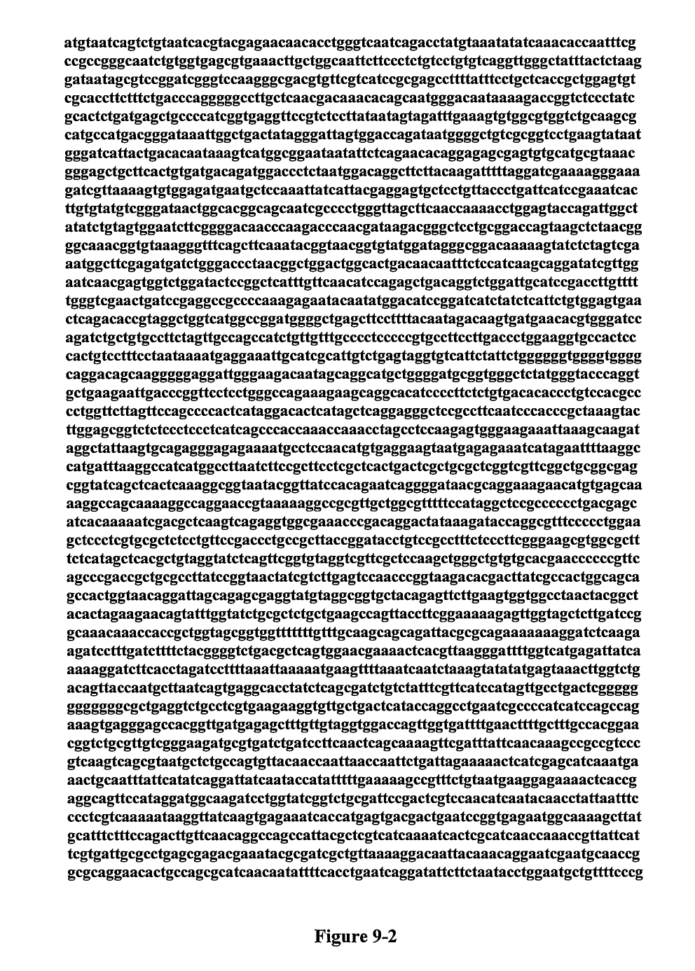

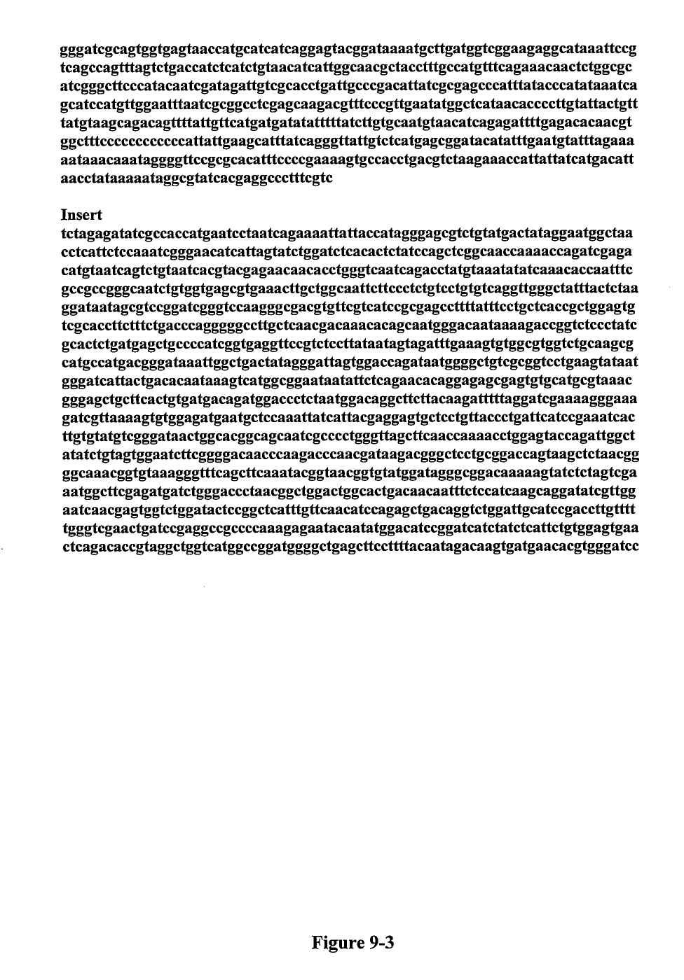

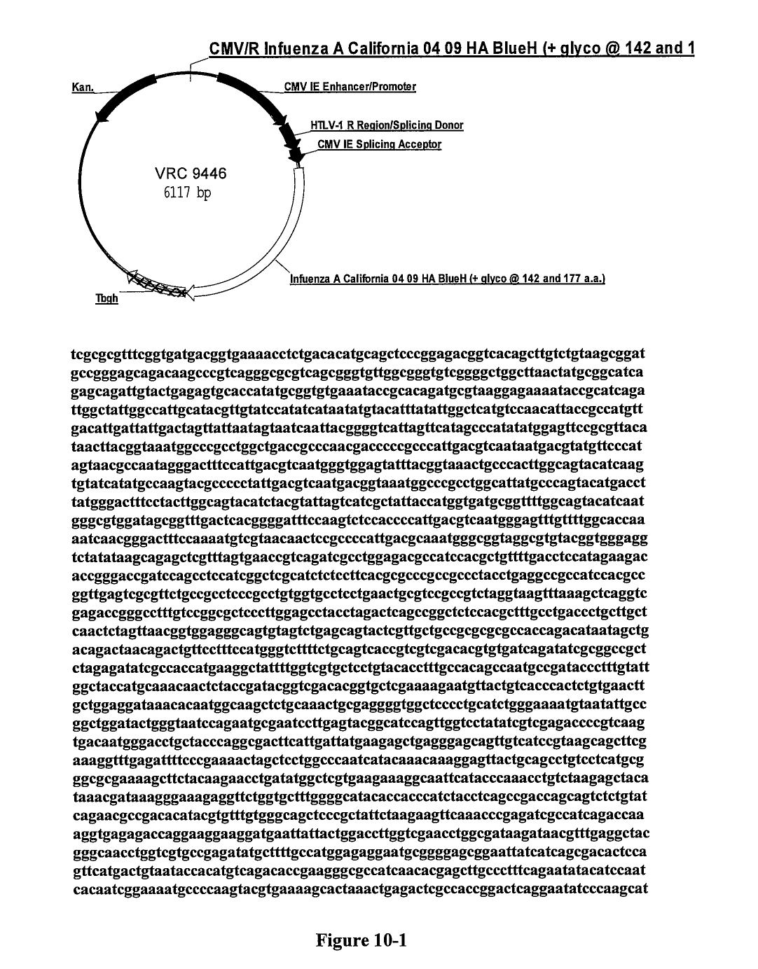

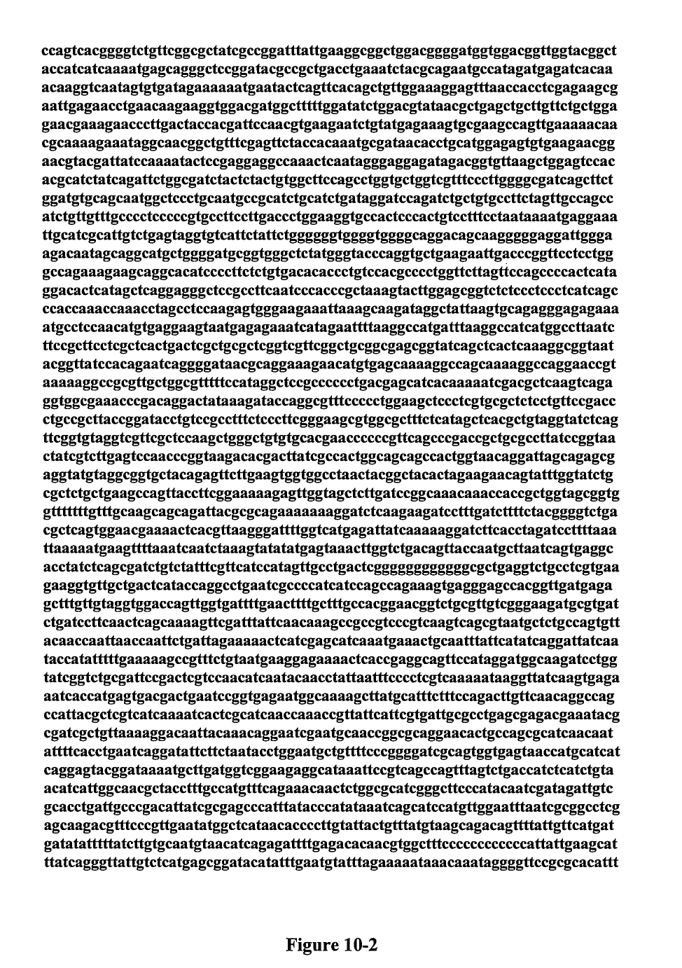

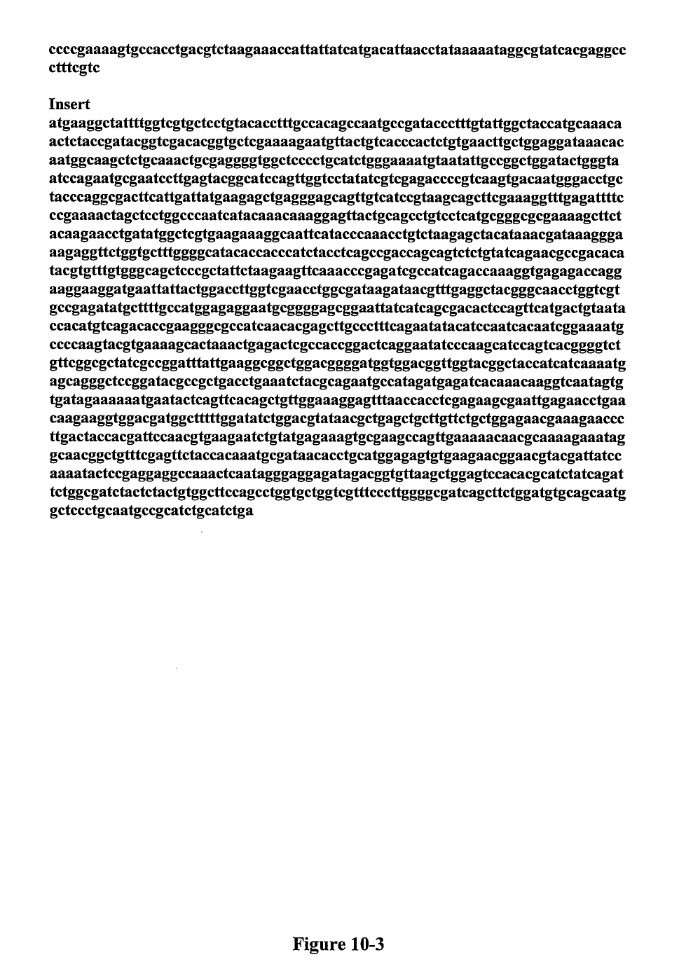

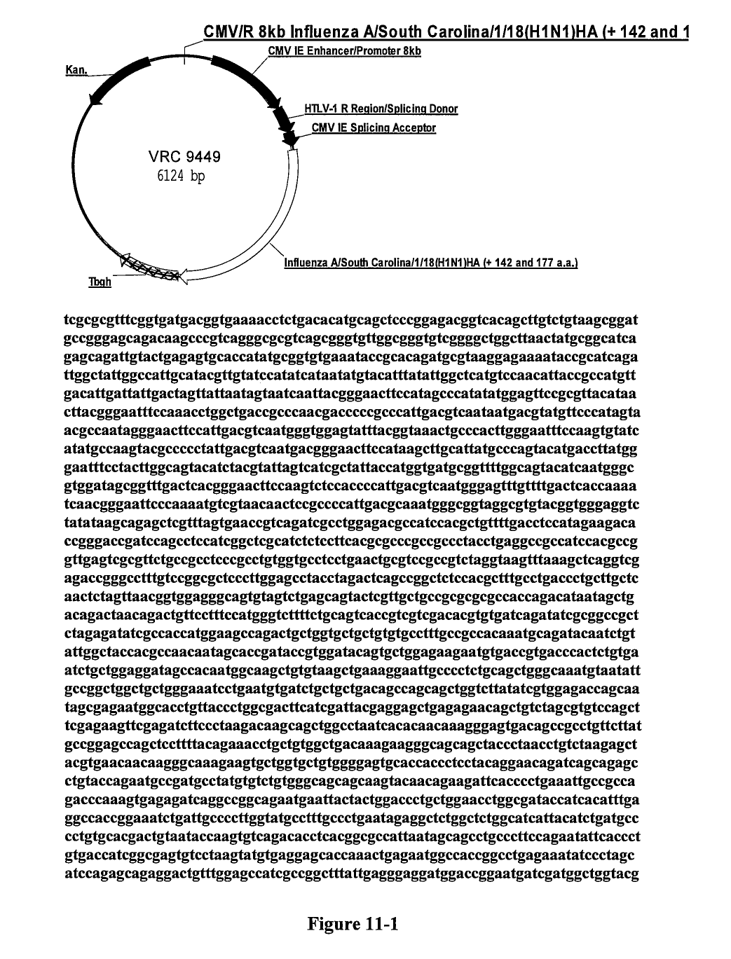

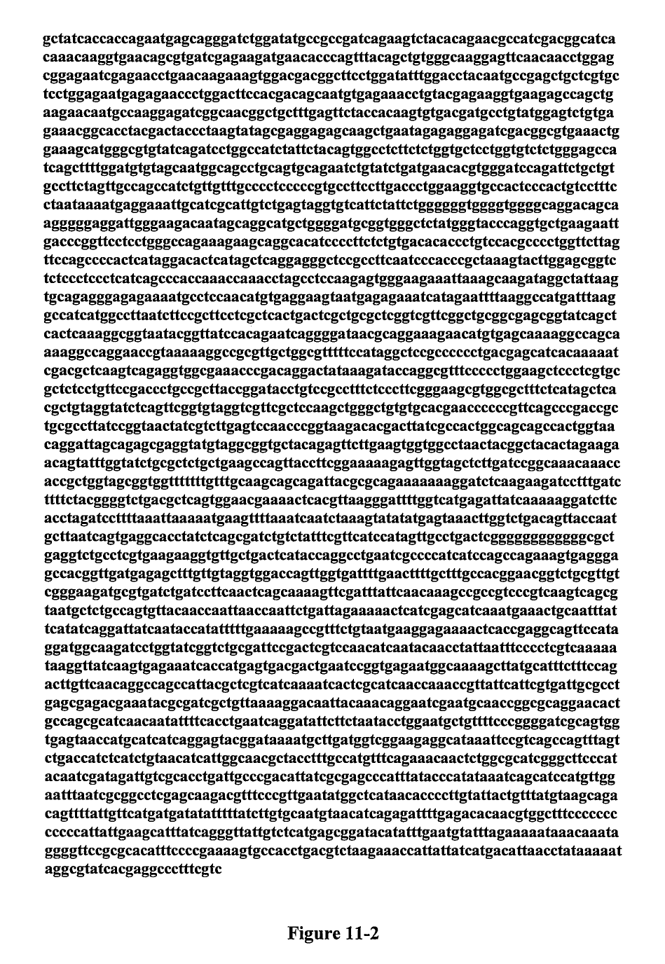

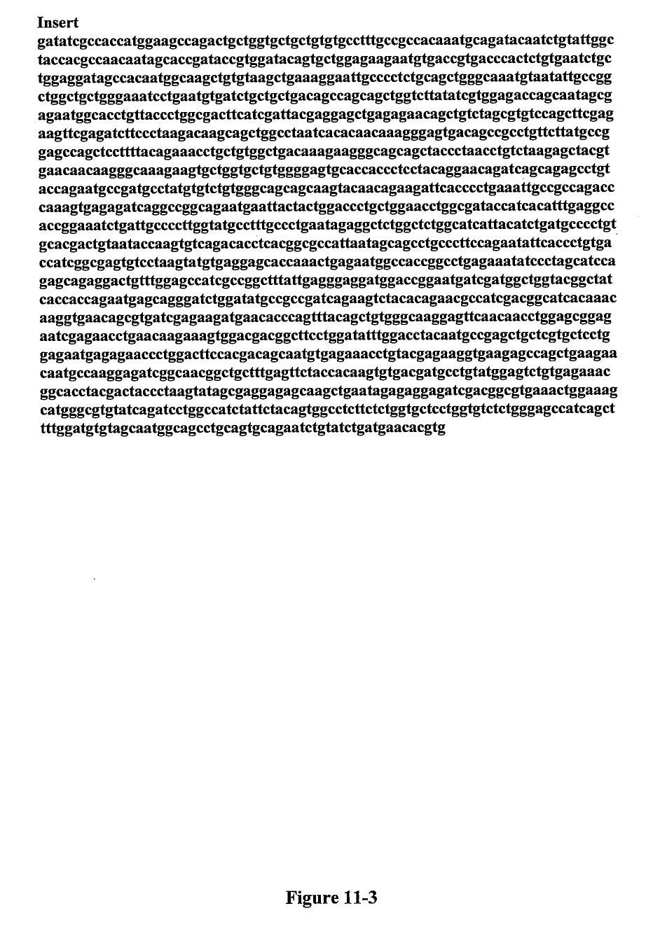

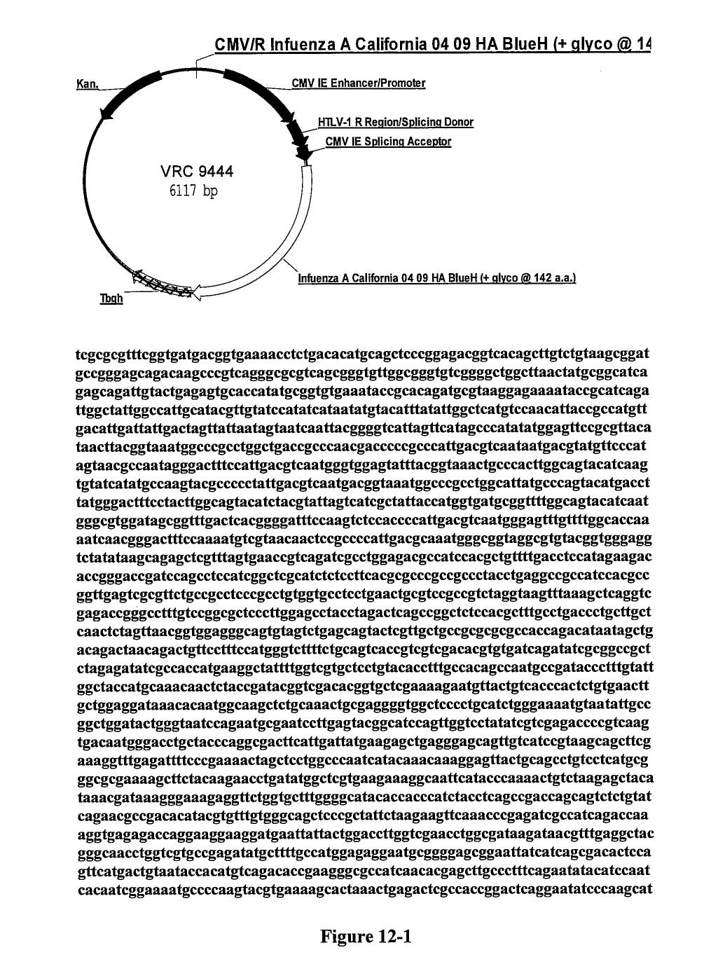

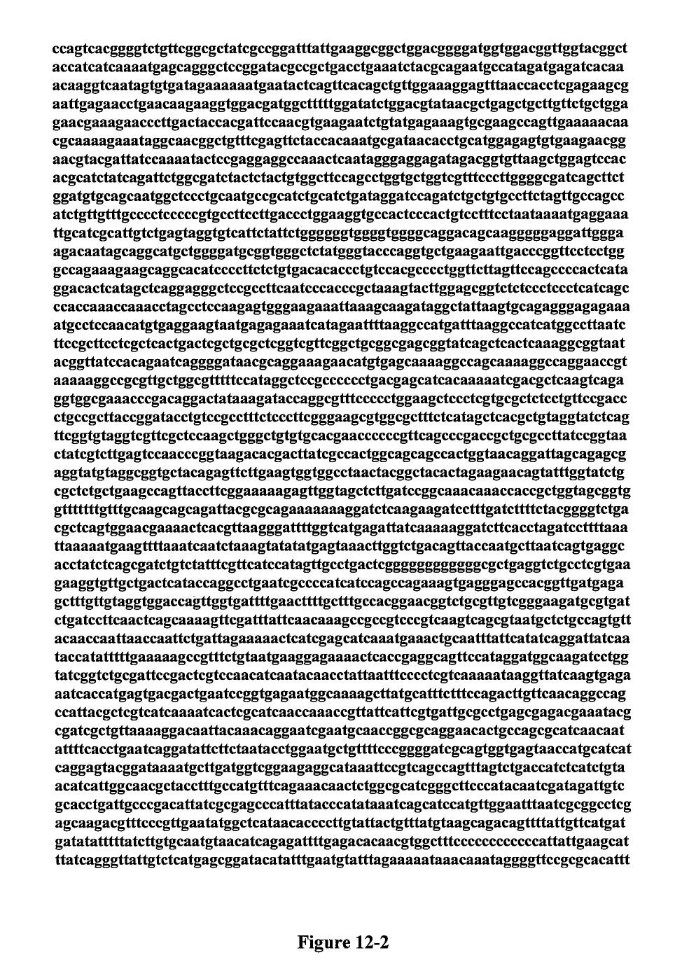

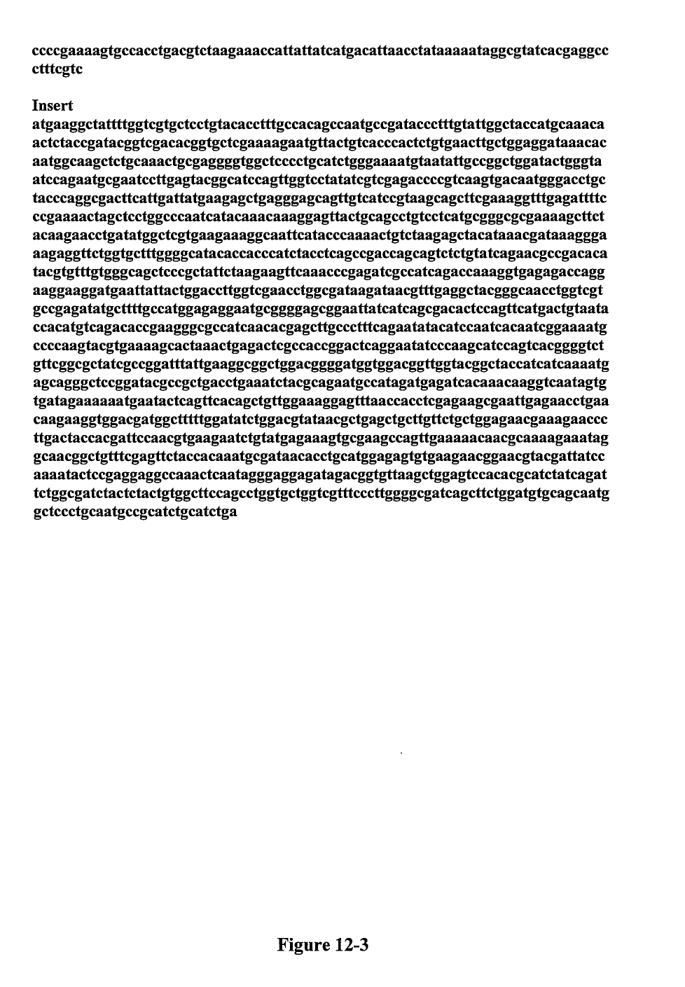

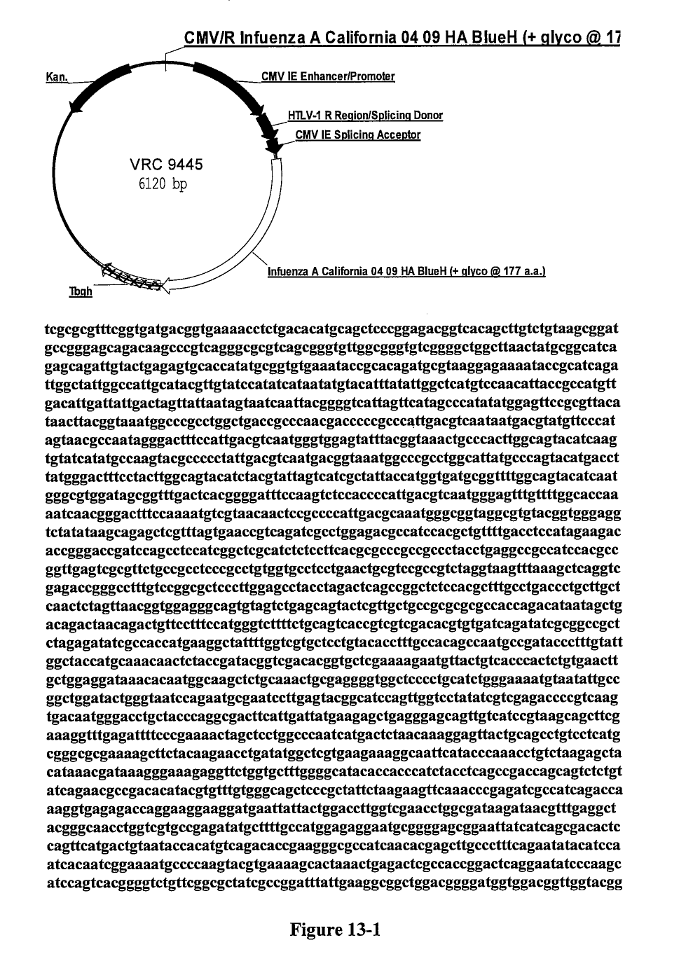

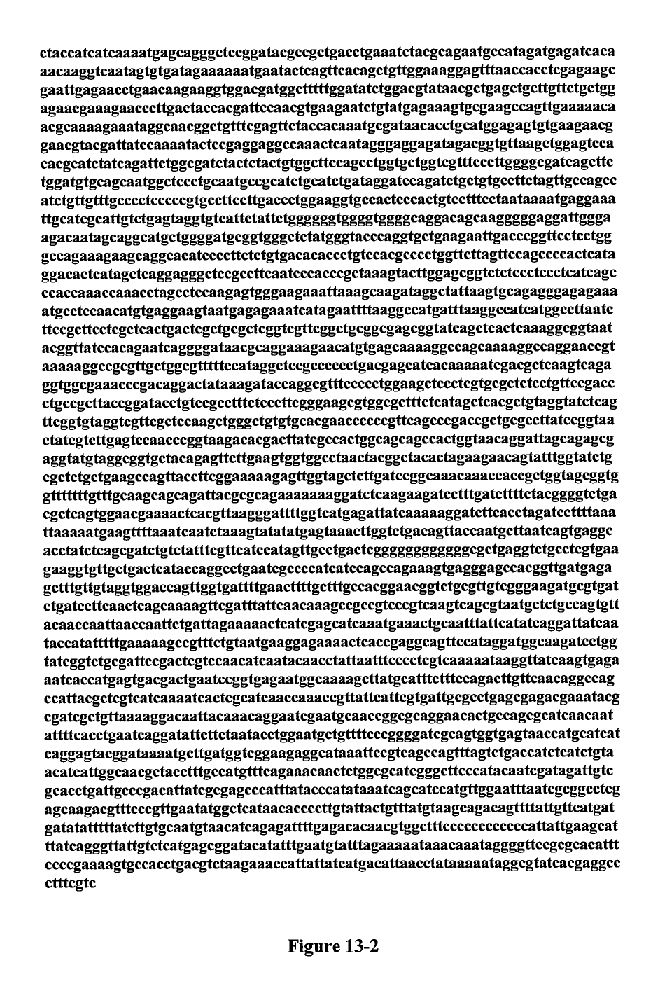

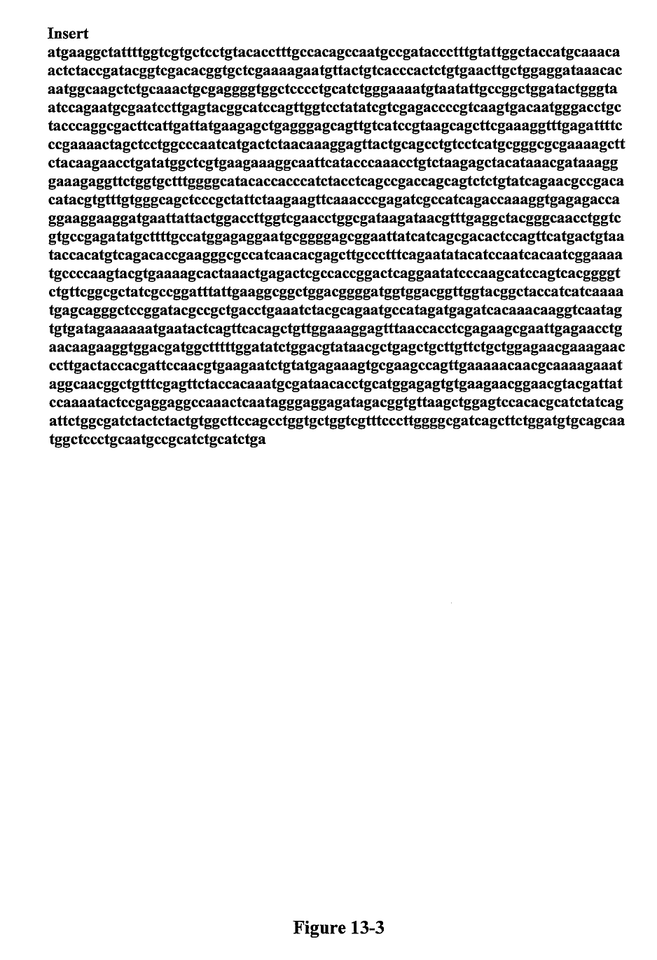

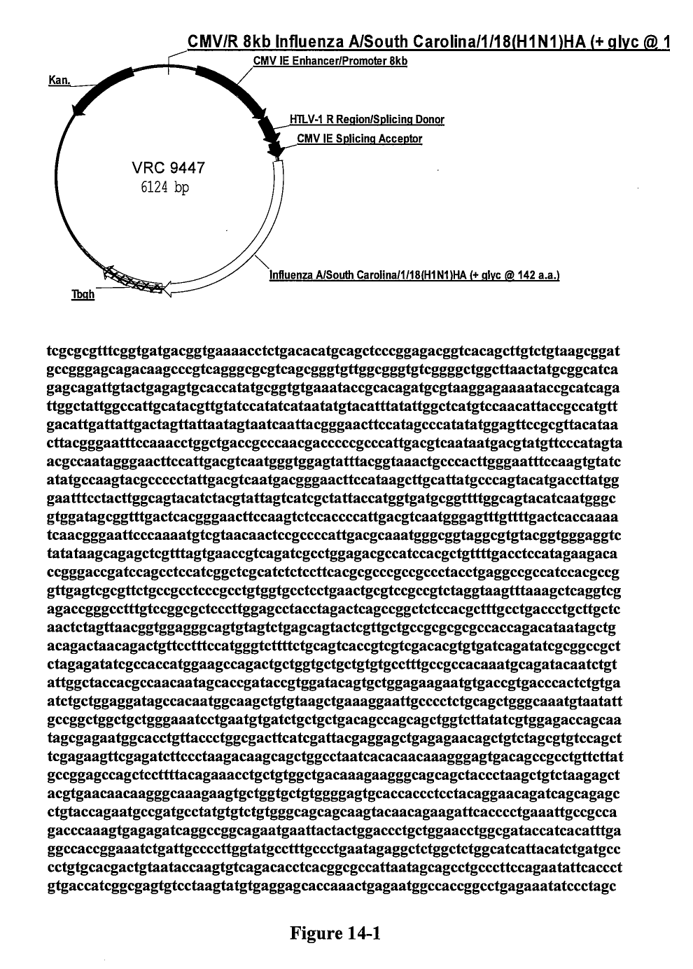

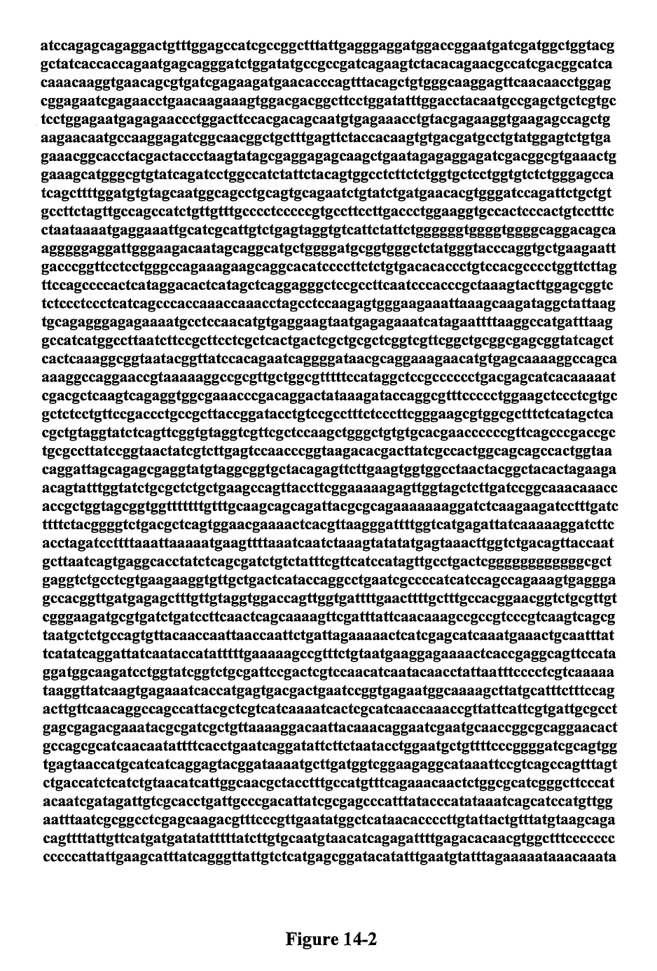

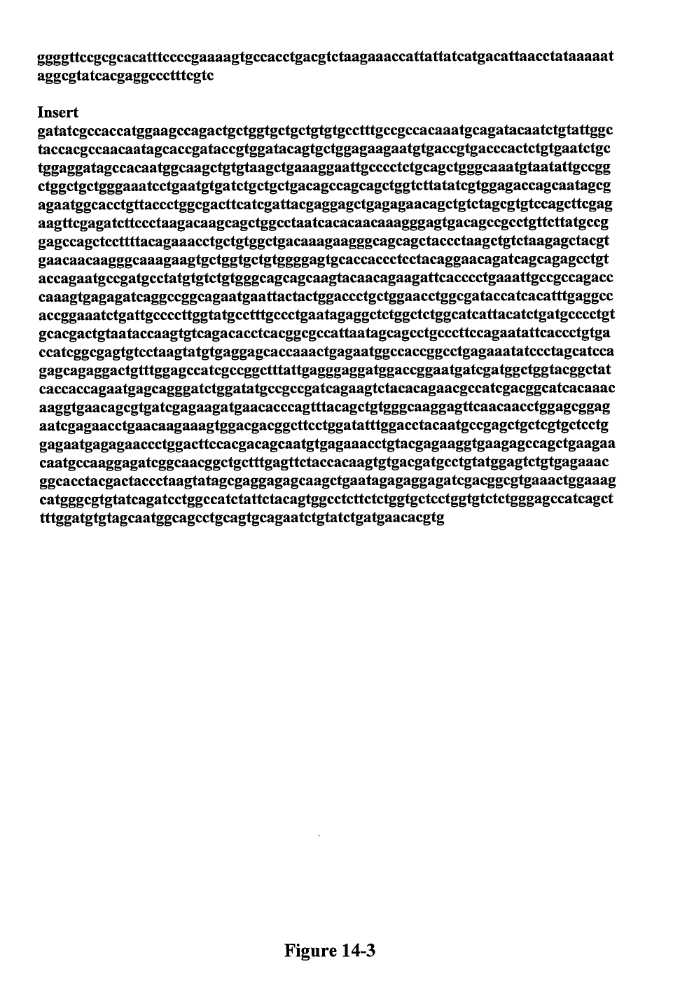

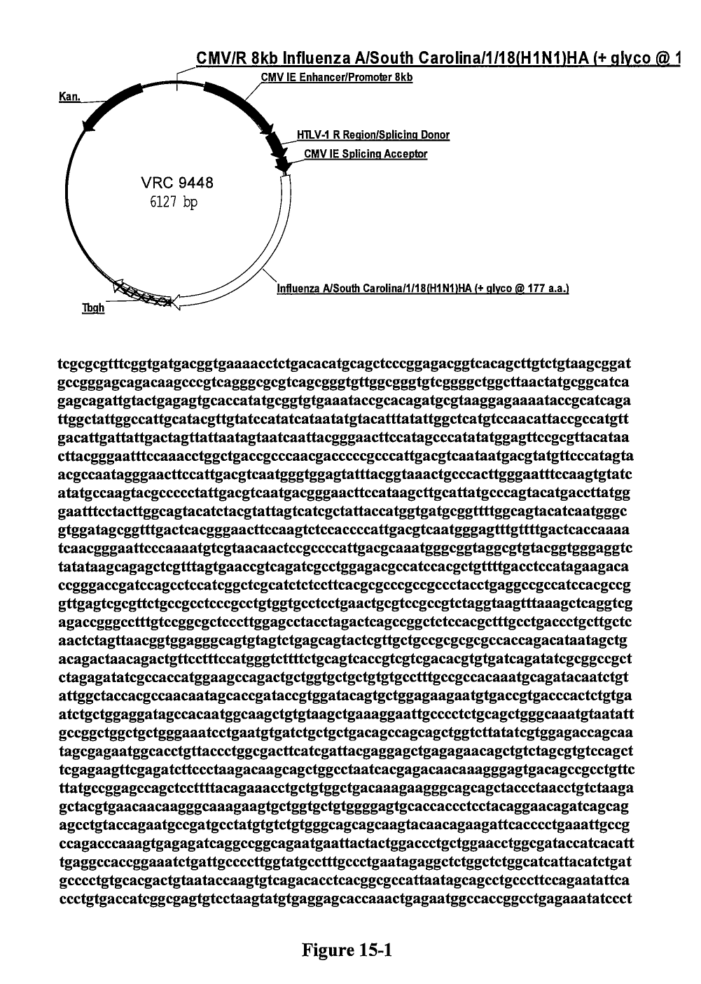

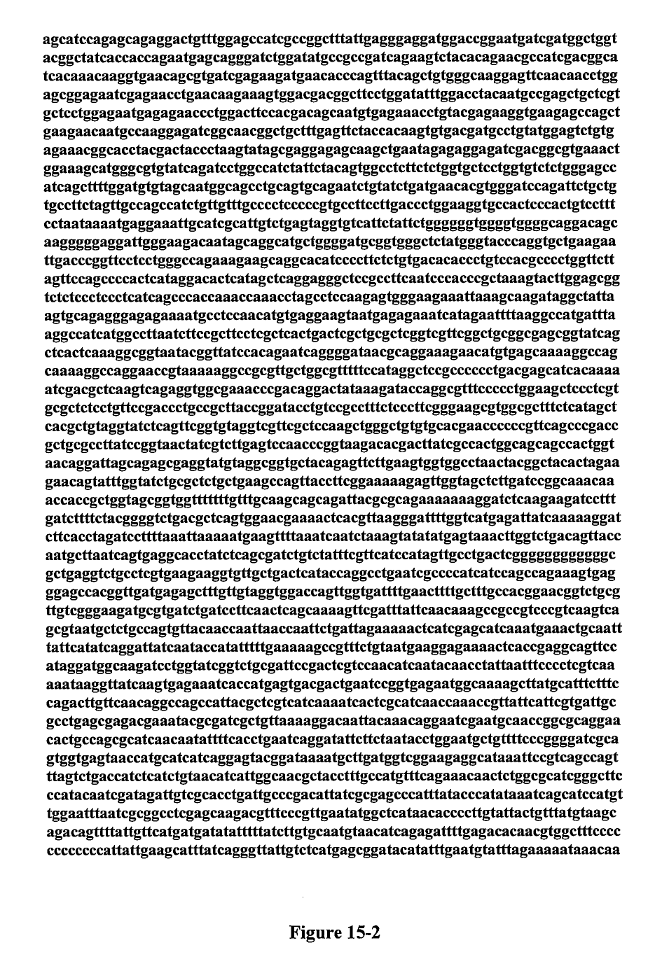

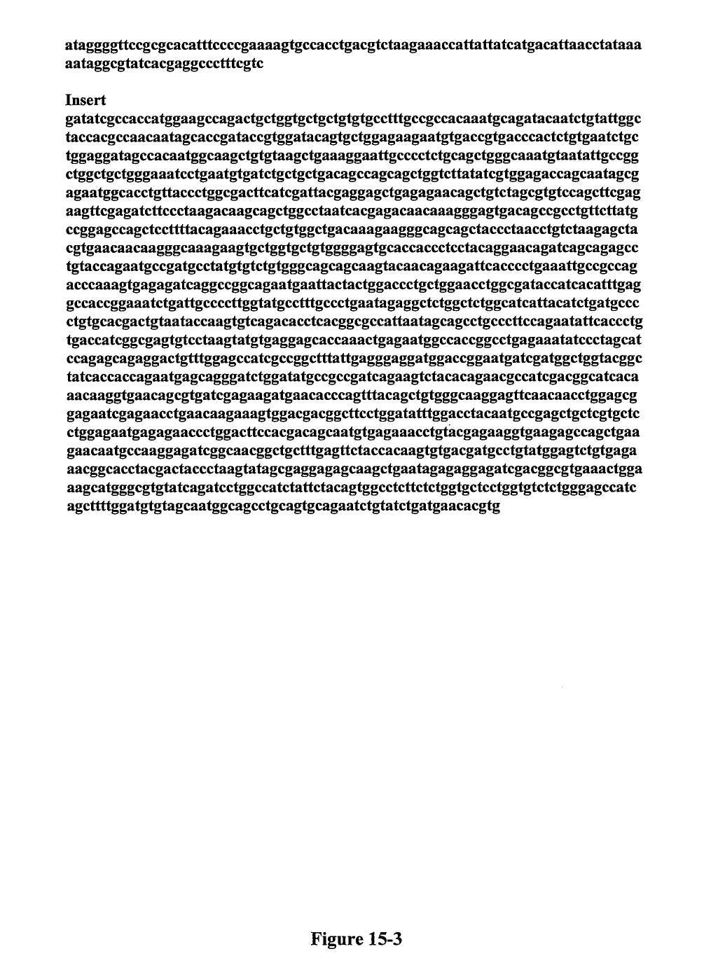

This Example demonstrates the role of glycans in protecting influenza virus against neutralization by antibodies. To confirm the role of the glycans described in the previous Examples, site-directed mutants were created that introduced glycosylation sites at amino acid positions 142 and 177 (1918 numbering) of 1918 SC and A (H1N1) 2009 (CA 04/09) hemagglutinin antigens. The resultant nucleic acid constructs were VRC 9449 (CMV/R 8 kb Influenza A/South Carolina/1/1918 (H1N1) HA [2G-142+177) and VRC 9446 (CMV/R Influenza A/California/04/2009 (H1N1) HA [2G-142+177]. The encoded HA proteins are also referred to as 1918 (2G) and 2009 (2G), respectively. Addition of the N-linked glycans to 1918 SC HA and 2009 CA HA proteins was confirmed by Endo H digestion and SDS-polyacrylamide gel electrophoresis (SDS-PAGE). Nucleic acid constructs encoding the ectodomain of wild-type (1918 and 2009) HA proteins, as well as nucleic acid constructs VRC 9449 and VRC 9446 (encoding 1918 (2G) and 2009 (2G), respectively, were expressed in 293F cells co-transfected with the relevant neuraminidase (NA), with or without the presence of swainsonine and kifunensine, to generate the physiologic trimer spike, as previously described (Wei, C. J. et al. Comparative efficacy of neutralizing antibodies elicited by recombinant hemagglutinin proteins from avian H₅N₁ influenza virus. J Virol. 82, 6200-6208 (2008)). Addition of the N-linked glycans to 1918 SC and 2009 CA HA proteins was confirmed by the increase in the size of the HA protein band, as observed by SDS-PAGE (FIG. 3A; band #1, compare 1918 to 1918 (2G), and 2009 to 2009 (2G)). HA proteins made in the presence of swainsonine and kifunensine were further treated with EndoH. Upon Endo H digestion (FIG. 3A; band #2), both wild-type HA and mutant HA proteins collapsed to the same size, indicating removal of complex carbohydrates and high mannose N-linked glycans. Addition of the N-linked glycans to 1918 SC HA and 2009 CA HA proteins was also confirmed by matrix-assisted laser desorption/ionization-mass spectrometry (MALDI-MS) (FIG. 3B).

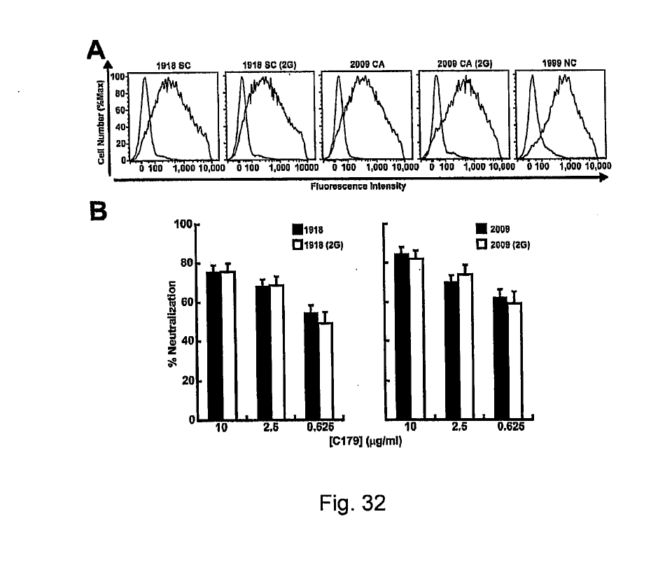

Comparable expression of the wild-type and mutant HA proteins was verified by transfecting nucleic acid constructs encoding the wild-type HA and mutant HA proteins into 293 cells along with the relevant NA using PROFECTION® Mammalian Transfection System (Promega, Madison, Wis.). Twenty four hours after transfection, cells were removed using PBS containing 2 mM EDTA. Cells were then washed 2× with cold PBS and transferred to a 96-well plate (0.5×10⁶ cells/well). Cells were incubated with C179 monoclonal antibody (at 5 ug/ml) [Okuno, Y., et al. A common neutralizing epitope conserved between the hemagglutinins of influenza A virus H1 and H2 strains. J Virol. 67, 2552-2558 (1993)] or purified naïve mouse IgG control for 30 minutes on ice, washed, and incubated with ALEXA FLUOR® 488 goat anti-mouse IgG (Invitrogen, Carlsbad, Calif.) (1:2000) for 30 minutes on ice. Cells were then washed 2 times with cold PBS and fixed with 0.5% paraformaldehyde, after which the samples were analyzed using an LSR cell analyzer (BD Biosciences, San Jose, Calif.) and Flow Jo software (Tree Star, Ashland, Oreg.). The results are shown in FIG. 32A. This analysis confirmed comparable expression of the wild-type HA and mutant HA proteins having two glycosylation sites (2G). In addition to the flow-cytometry analysis, the ability of the C179 antibody to neutralize pseudotyped viruses containing either wild-type or mutant HA was measured. The results of this analysis, which are shown in FIG. 32B, demonstrate that input wild-type and glycosylation mutant pseudotyped viruses were neutralized by C179 to similar degrees (compare 1918 to 1918 (2G) and 2009 to 2009 (2G)).

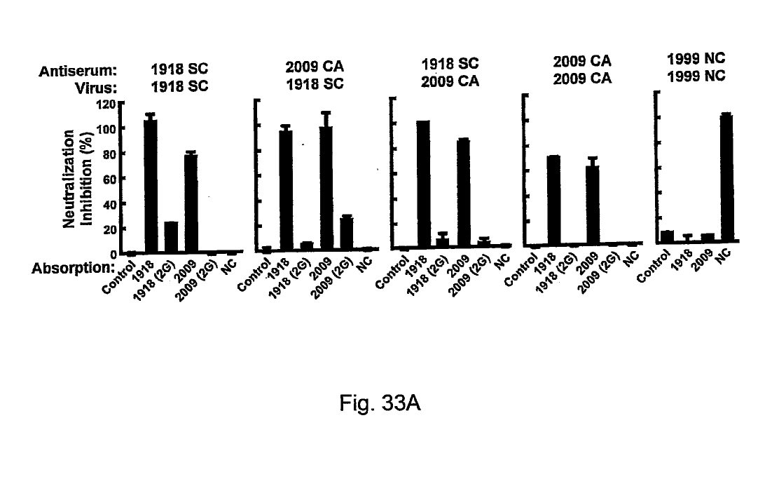

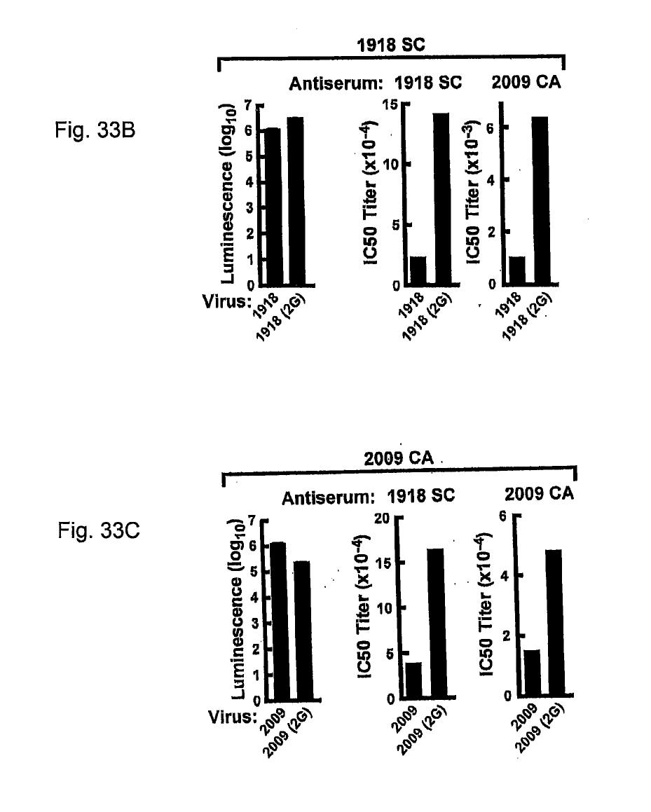

Wild-type and mutant HA proteins were tested for their ability to affect neutralization of pseudotyped 1918 SC, 2009 CA and 1999 NC viruses. Neutralization assays were performed as described in Example 1. To measure the ability of glycosylated HA protein to affect neutralization by antisera, mouse antisera was diluted in 250 μl of culture medium and incubated for 30 minutes with 8×10⁶ 293F cells that had been transfected with nucleic acid constructs encoding either the wild-type HA or mutant HA protein and the relevant NA. The pre-absorbed sera were then collected and used for the neutralization assay, the results of which are shown in FIG. 33A. Absorption of antiserum with cells expressing wild-type 1918 or A (H1N1) 2009 HA protein removed the neutralizing antibody activity against both pandemic viruses (FIG. 33A, 1918 and 2009). In contrast, when the RBD glycosylation sites were introduced into the HA trimer, or when seasonal 1999 NC was used, the mutant HA proteins failed to absorb these neutralizing antibodies (FIG. 33A; 1918 (2G), 2009 (2G), NC). The seasonal 1999 NC HA protein effectively absorbed homologous neutralizing antibodies (FIG. 33A, far right panel).

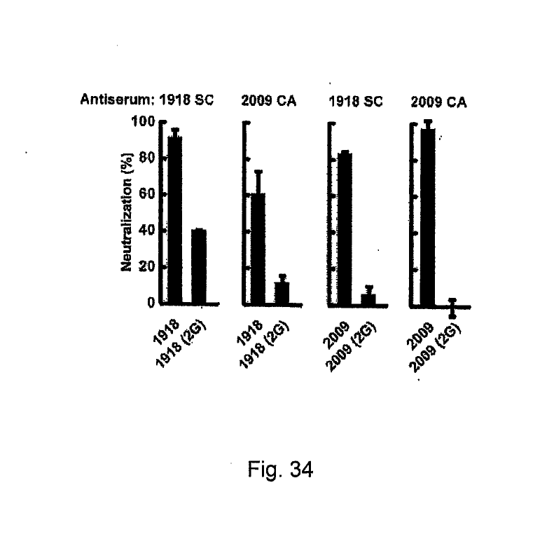

The ability of the wild-type and mutant HA proteins to mediate viral entry into the cell was also investigated. Both the wild type and mutant 1918 SC and A (H1N1) 2009 HA proteins were similar in their ability to mediate gene transfer using pseudotyped lentiviral reporters (FIGS. 33B and C, left panels), showing that glycosylation did not compromise trimer function. The neutralization sensitivity of the glycosylated mutant reporters was then assessed by incubation of the mutant pseudotyped reporter viruses with antisera to 1918 SC or 2009 CA. Incubation of the glycosylated mutant reporters with antisera to 1918 SC or A (H1N1) 2009 greatly increased the concentration of antibody needed to inhibit entry of virus by 50% (that is, a lower IC₅₀ (median inhibitory concentration) titer. Therefore, the 1918 (2G) and A (H1N1) 2009 (2G) mutants were markedly resistant to neutralization, compared to their wild type counterparts [FIGS. 33B and C, middle and right panels, 1918 vs. 1918 (2G); 2009 vs. 2009 (2G); see also FIG. 34]. The glycan at amino acid 142 was largely responsible for neutralization resistance [see Table 5; 1918 SC (1G-142)]. In contrast, 1999 NC with glycosylation at either position 142 or 177 was sensitive to homologous neutralization but resistant to neutralization by 1918 SC or 2009 CA antisera. [Table 5; 1999 NC (1G-142) and (1G-177)]. Because the RBD-A region has only 58% to 67% sequence identity between seasonal and pandemic strains, 1918 SC or 2009 CA antisera would likely not neutralize seasonal strains, even without glycosylation. Neither 1918 SC nor 2009 CA antiserum neutralized A/PR/8/1934 (1934 PR8), a strain with one glycosylation site on the head region of the HA protein (144, 1918 SC numbering) (Table 5). Deglycosylation of the 1934 PR8 HA protein at that position did not confer complete sensitivity to 1918- or 2009-neutralizing antibodies [Table 5, 1934 PR8 (N144Q)]

TABLE 5

| | Virus | | | | | | | | |
|---|---|---|---|---|---|---|---|---|---|
| | | 1918 | 1918 | 1918 | | 1934 | | 1999 | 1999 |
| | 1918 | SC | SC | SC | 1934 | PR8 | 1999 | NC | NC |
| Immune sera | SC | (IG-142) | (IG-177) | (2G) | PR8 | (N144Q) | NC | (IG-142) | (IG-177) |
| 1918 SC | 4520 | 1073 | 3790 | 710 | 0 | 0 | <50 | 0 | 0 |
| 1934 PR8 | 0 | ND^{a} | ND | ND | 1499 | 1031 | ND | ND | ND |
| 1999 NC | 0 | ND | ND | ND | ND | ND | 1698 | 711 | 1795 |
| 2009 CA | 994 | 150 | 754 | 158 | 0 | <50 | 0 | 0 | 0 |

### Example 5

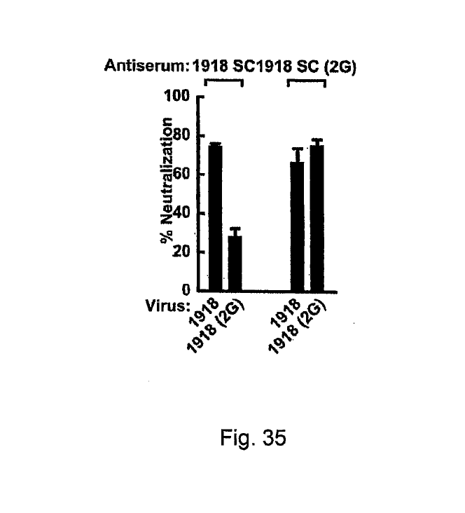

To explore the efficacy of glycan-modified and wild-type HA as vaccine immunogens, mice were immunized with nucleic acid constructs encoding 1918 (SC) HA [VRC 7730; SEQ ID NO:1] or 1918 SC (2G) HA [VRC 9449; SEQ ID NO:29] protein. The result of this study is shown in FIG. 35. Antisera from mice immunized with a nucleic acid construct encoding wild-type 1918 SC HA neutralized homologous virus but poorly inhibited the (2G) derivative. In contrast, 1918 SC (2G) immune sera neutralized both wild-type 1918 SC and mutant 1918 SC (2G) viruses. These data demonstrate that immunization with a nucleic acid construct encoding the RBD-A glycosylated HA (i.e., HA with a glycan-shielded RBD-A region) confers improved protection against a glycosylated variant that might evolve into a seasonal form of influenza. Immunization with such a nucleic acid construct also neutralizes the pandemic virus.

### Example 6

This Example describes in vivo testing of hemagglutinin antigens having one or two glycosylation sites in the RBD-A region.

CMV/R-based nucleic acid constructs comprising nucleic acid molecules encoding each of the following hemagglutinin antigens were produced as described herein.

| SEQ ID NO | Hemagglutinin antigen (HA) |
|---|---|
| SEQ ID NO: | A/Haishu/SWL110/2010/ADG21188 (H1N1) HA (which |
| 69 | includes an N-linked glycosylation site at AA 179) |
| SEQ ID NO: | A/Netherlands/1493b/2009/ADJ40554 (H1N1) HA (which |
| 73 | includes an N-linked glycosylation site at AA 136) |
| SEQ ID NO: | A/Orenburg/IIV-13/2010/ADF42661 (H1N1) HA (which |
| 77 | includes N-linked glycosylation sites at AA 136 and AA |
| | 179) |
| SEQ ID NO: | A/Orenburg/IIV-13/2010/AD199498 (H1N1) HA (which |
| 81 | includes an N-linked glycosylation site at AA 179) |
| SEQ ID NO: | A/Russia/178/2009/ADA79597 (H1N1) HA (which |
| 85 | includes an N-linked glycosylation site at AA 179) |
| SEQ ID NO: | A/Russia/180/2009/ADB81459 (H1N1) HA (which |
| 89 | includes an N-linked glycosylation site at AA 179) |
| SEQ ID NO: | A/Salekard/01/2009/ADA83044 (H1N1) HA (which |
| 93 | includes an N-linked glycosylation site at AA 179) |
| SEQ ID NO: | A/Tallinn/INS183/2010/ADG42553 (H1N1) HA (which |
| 97 | includes an N-linked glycosylation site at AA 179) |
| SEQ ID NO: | A/Beijing/SE2649/2009/ADD64214 (H1N1) HA (which |
| 101 | includes an N-linked glycosylation site at AA 179) |
| SEQ ID NO: | A/California/VRDL6/2010/ADI99550 (H1N1) HA (which |
| 105 | includes an N-linked glycosylation site at AA 179) |

Maps and nucleic acid sequences of these nucleic acid constructs are presented in the Figures; the nucleic acid sequences of these constructs are, respectively, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, and SEQ ID NO:103.

The encoded hemagglutinin antigens have RBD-A regions homologous to those of pandemic influenza A/California/04/2009 (H1N1) HA except that the listed HAs have N-linked glycosylation sites as indicated. In view of having such glycosylation sites, the viruses from which these HAs are derived are thought to be evolving influenza A subtype H1 viruses. That is, they apparently are evolving from a pandemic strain into a seasonal strain: without being bound by theory, it is believed that mutations to encode glycosylation sites in the RBD-A region are occurring in order to evade an immune response directed against pandemic virus. It is to be appreciated that other mutations encoding different amino acids can also be part of an evasion mechanism.

The nucleic acid constructs are administered to mice as described in Example 1. Antisera are isolated from the mice as described in Example 1 and tested for their abilities to neutralize various influenza virus using methods as described herein. Virus that can be tested include homologous virus, pandemic virus, such as A/California/04/2009 (H1N1), other apparently-evolving virus, and seasonal virus strains. These antisera can also be compared to antisera isolated from mice administered a glycosylated-shielded immunogen, such as VRC 9449, a nucleic acid construct encoding 1918 SC [2G-142+177] hemagglutinin antigen.

The following table is provided as a reference to the sequences disclosed in this application.

TABLE 6
Sequence Reference Table

| | HA Protein | Nucleic | |
|---|---|---|---|
| | GenBank | Acid | |
| SEQ ID NO | Accession No. | Construct | Name/Description |
| 1 | | VRC 7730 | CMV/R 8 kb Influenza A/South Carolina/1/1918 |
| | | | (H1N1) HA-wt |
| 2 | | | Coding sequence |
| 3 | AAD17229 | | Translation of SEQ ID NO: 2 |
| 4 | | | Inverse Complement of SEQ ID NO: 2 |
| 5 | | VRC 7733 | CMV/R 8 kb Influenza A/South Carolina/1/1918 |
| | | | (H1N1) HA mut A short foldon-his |
| 6 | | | Coding sequence |
| 7 | AAD17229 | | Translation of SEQ ID NO: 6 |
| 8 | | | Inverse Complement of SEQ ID NO: 6 |
| 9 | | VRC 9259 | CMV/R 8 kb Influenza A/Brevig Mission/1/1918 |
| | | | (H1N1) NA/h |
| 10 | | | Coding sequence |
| 11 | AAF77036 | | Translation of SEQ ID NO: 10 |
| 12 | | | Inverse Complement of SEQ ID NO: 10 |
| 13 | | VRC 7764 | CMV/R 8 kb Influenza A/New Caledonia/20/1999 |
| | | | (H1N1) HA foldon-his |
| 14 | | | Coding sequence |
| 15 | AAP34324 | | Translation of SEQ ID NO: 14 |
| 16 | | | Inverse Complement of SEQ ID NO: 14 |
| 17 | | VRC 9450 | CMV/R Influenza A/California/04/2009 (H1N1) HA |
| | | | foldon His |
| 18 | | | Coding sequence |
| 19 | | | Translation of SEQ ID NO: 18 |
| 20 | | | Inverse Complement of SEQ ID NO: 18 |
| 21 | | VRC 9451 | CMV/R Influenza A H1N1 California/4/2009 |
| | | | NA_BlueH |
| 22 | | | Coding sequence |
| 23 | | | Translation of SEQ ID NO: 22 |
| 24 | | | Inverse Complement of SEQ ID NO: 22 |
| 25 | | VRC 9446 | CMV/R Influenza A/California/04/2009 (H1N1) HA/h |
| | | | BlueH w/glycosylation at AA 142 and AA 177 |
| | | | (encodes 2009 CA [2G-142 + 177] HA) |
| 26 | | | Coding sequence |
| 27 | | | Translation of SEQ ID NO: 26 |
| 28 | | | Inverse Complement of SEQ ID NO: 26 |
| 29 | | VRC 9449 | CMV/R 8 kb Influenza A/South Carolina/1/1918 |
| | | | (H1N1) HA w/glycosylation at AA 142 and AA 177 |
| | | | (encodes 1918 SC [2G-142 + 177] HA) |
| 30 | | | Coding sequence |
| 31 | | | Translation of SEQ ID NO: 30 |
| 32 | | | Inverse Complement of SEQ ID NO: 30 |
| 33 | | VRC 9444 | CMV/R Influenza A/California/04/2009 (H1N1) HA/h |
| | | | BlueH w/glycosylation at AA 142 (encodes 2009 CA |
| | | | [1G-142] HA) |
| 34 | | | Coding sequence |
| 35 | | | Translation of SEQ ID NO: 34 |
| 36 | | | Inverse Complement of SEQ ID NO: 34 |
| 37 | | VRC 9445 | CMV/R Influenza A/California/04/2009 (H1N1) HA/h |
| | | | BlueH w/glycosylation at AA 177 (encodes 2009 CA |
| | | | [1G-177] HA) |
| 38 | | | Coding sequence |
| 39 | | | Translation of SEQ ID NO: 38 |
| 40 | | | Inverse Complement of SEQ ID NO: 38 |
| 41 | | VRC 9447 | CMV/R 8 kb Influenza A/South Carolina/1/1918 |
| | | | (H1N1) HA w/glycosylation at AA 142 (encodes 1918 |
| | | | SC [1G-142] HA) |
| 42 | | | Coding sequence |
| 43 | | | Translation of SEQ ID NO: 42 |
| 44 | | | Inverse Complement of SEQ ID NO: 42 |
| 45 | | VRC 9448 | CMV/R 8 kb Influenza A/South Carolina/1/1918 |
| | | | (H1N1) HA w/glycosylation at AA 177 (encodes 1918 |
| | | | SC [1G-177] HA) |
| 46 | | | Coding sequence |
| 47 | | | Translation of SEQ ID NO: 46 |
| 48 | | | Inverse Complement of SEQ ID NO: 46 |
| 49 | AAD17229 | | Influenza A/South Carolina/1/1918 (H1N1) HA |
| 50 | FJ966082* | | Influenza A/California/04/2009 (H1N1) HA nucleic |
| | *nucleic acid | | acid sequence |
| | sequence | | |
| 51 | ACT83741 | | Influenza A/Ancona/05/2009 (H1N1) HA |
| 52 | ACT68118 | | Influenza A/Canada/MB/RV2013/2009 (H1N1) HA |
| 53 | ACT79133 | | Influenza A/Japan/1070/2009 (H1N1) HA |
| 54 | ACQ89903 | | Influenza A/Mexico/InDRE4114/2009 (H1N1) HA |
| 55 | ACS45017 | | Influenza A/Nanjing/1/2009 (H1N1) HA |
| 56 | ACP44147 | | Influenza A/New York/19/2009 (H1N1) HA |
| 57 | ACS91398 | | Influenza A/Paris/2722/2009 (H1N1) HA |
| 58 | ACR78160 | | Influenza A/Perth/29/2009 (H1N1) HA |
| 59 | ACT82516 | | Influenza A/Sao Paulo/43812/2009 (H1N1) HA |
| 60 | ACT21932 | | Influenza A/Stockholm/31/2009 (H1N1) HA |
| 61 | ACP41934 | | Influenza A/Texas/05/2009 (H1N1) HA |
| 62 | ACP41105 | | Influenza A/California/04/2009 (H1N1) HA |
| 63 | | VRC 9328 | CMV/R Influenza A/California/04/2009 (H1N1) HA |
| | | | BlueH |
| 64 | | | Coding sequence |
| 65 | ACP41105 | | Translation of SEQ ID NO: 64 |
| 66 | | | Inverse Complement of SEQ ID NO: 64 |
| 67 | | | CMV/R Influenza |
| | | | A/Haishu/SWL110/2010/ADG21188 (H1N1) HA |
| | | | (which includes an N-linked glycosylation site at AA |
| | | | 179) |
| 68 | | | Coding sequence |
| 69 | ADG21188 | | Translation of SEQ ID NO: 68 |
| 70 | | | Inverse Complement of SEQ ID NO: 68 |
| 71 | | | CMV/R Influenza |
| | | | A/Netherlands/1493b/2009/ADJ40554 (H1N1) HA |
| | | | (which includes an N-linked glycosylation site at AA |
| | | | 136) |
| 72 | | | Coding sequence |
| 73 | ADJ40554 | | Translation of SEQ ID NO: 72 |
| 74 | | | Inverse Complement of SEQ ID NO: 72 |
| 75 | | | CMV/R Influenza A/Orenburg/IIV- |
| | | | 13/2010/ADF42661 (H1N1) HA (which includes N- |
| | | | linked glycosylation sites at AA 136 and AA 179) |
| 76 | | | Coding sequence |
| 77 | ADF42661 | | Translation of SEQ ID NO: 76 |
| 78 | | | Inverse Complement of SEQ ID NO: 76 |
| 79 | | | CMV/R Influenza A/Orenburg/IIV-13/2010/AD199498 |
| | | | (H1N1) HA (which includes an N-linked glycosylation |
| | | | site at AA 179) |
| 80 | | | Coding sequence |
| 81 | AD199498 | | Translation of SEQ ID NO: 80 |
| 82 | | | Inverse Complement of SEQ ID NO: 80 |
| 83 | | | CMV/R Influenza A/Russia/178/2009/ADA79597 |
| | | | (H1N1) HA (which includes an N-linked glycosylation |
| | | | site at AA 179) |
| 84 | | | Coding sequence |
| 85 | ADA79597 | | Translation of SEQ ID NO: 84 |
| 86 | | | Inverse Complement of SEQ ID NO: 84 |
| 87 | | | CMV/R Influenza A/Russia/180/2009/ADB81459 |
| | | | (H1N1) HA (which includes an N-linked glycosylation |
| | | | site at AA 179) |
| 88 | | | Coding sequence |
| 89 | ADB81459 | | Translation of SEQ ID NO: 88 |
| 90 | | | Inverse Complement of SEQ ID NO: 88 |
| 91 | | | CMV/R Influenza A/Salekard/01/2009/ADA83044 |
| | | | (H1N1) HA (which includes an N-linked glycosylation |
| | | | site at AA 179) |
| 92 | ADA83044 | | Coding sequence |
| 93 | | | Translation of SEQ ID NO: 92 |
| 94 | | | Inverse Complement of SEQ ID NO: 92 |
| 95 | | | CMV/R Influenza A/Tallinn/INS183/2010/ADG42553 |
| | | | (H1N1) HA (which includes an N-linked glycosylation |
| | | | site at AA 179) |
| 96 | | | Coding sequence |
| 97 | ADG42553 | | Translation of SEQ ID NO: 96 |
| 98 | | | Inverse Complement of SEQ ID NO: 96 |
| 99 | | | CMV/R Influenza A/Beijing/SE2649/2009/ADD64214 |
| | | | (H1N1) HA (which includes an N-linked glycosylation |
| | | | site at AA 179) |
| 100 | | | Coding sequence |
| 101 | ADD64214 | | Translation of SEQ ID NO: 100 |
| 102 | | | Inverse Complement of SEQ ID NO: 100 |
| 103 | | | CMV/R Influenza |
| | | | A/California/VRDL6/2010/ADI99550 (H1N1) HA |
| | | | (which includes an N-linked glycosylation site at AA |
| | | | 179) |
| 104 | | | Coding sequence |
| 105 | ADI99550 | | Translation of SEQ ID NO: 104 |
| 106 | | | Inverse Complement of SEQ ID NO: 104 |
| 107 | | | CMV/R 8 kb Influenza H1 (A/PR8/8/1934) (H1N1) |
| | | | HA/h N144Q |
| 108 | | | Coding sequence |
| 109 | | | Translation of SEQ ID NO: 108 |
| 110 | | | Inverse Complement of SEQ ID NO: 108 |
| 111 | | | CMV/R 8 kb Influenza A/New Caledonia/20/1999 |
| | | | (H1N1) HA wt N142Q |
| 112 | | | Coding sequence |
| 113 | | | Translation of SEQ ID NO: 112 |
| 114 | | | Inverse Complement of SEQ ID NO: 112 |
| 115 | | | CMV/R 8 kb Influenza A/New Caledonia/20/1999 |
| | | | (H1N1) HA wt N177Q |
| 116 | | | Coding sequence |
| 117 | | | Translation of SEQ ID NO: 116 |
| 118 | | | Inverse Complement of SEQ ID NO: 116 |
| 119 | | VRC 9162 | CMV/R Influenza A/New Caledonia/20/1999 (H1N1) |
| | | | NA |
| 120 | | | Coding sequence |
| 121 | | | Translation of SEQ ID NO: 120 |
| 122 | | | Complement of SEQ ID NO: 120 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

1. An immunogen comprising a nucleic acid construct comprising a nucleic acid molecule encoding a protein comprising an influenza A subtype H1 hemagglutinin glycan-shielded receptor binding domain A (RBD-A) region and at least one influenza A subtype H1 hemagglutinin antigenic site selected from the group consisting of an HA1 globular head antigenic site and an HA2 antigenic site, wherein said antigenic site is not within the RBD-A region,
wherein said antigenic site elicits the production of neutralizing antibodies against an antigenic site of a pandemic influenza A subtype H1 HA, and
wherein said glycan-shielded RBD-A region is homologous to the RBD-A region of said pandemic influenza A subtype H1 HA, with the exception that said glycan-shielded RBD-A region comprises at least one N-linked glycosylation site and said pandemic RBD-A region lacks any N-glycosylation sites.

2-3. (canceled)

4. The immunogen of claim 1, wherein said glycan-shielded RBD-A region is at least 80% identical to said pandemic RBD-A region.

5. (canceled)

6. The immunogen of claim 1, wherein the encoded protein is selected from the group consisting of:
(a) a protein in which the glycan-shielded RBD-A region of said protein comprises an RBD-A region of an HA having an amino acid sequence selected from the group consisting of SEQ ID NO:27, SEQ ID NO:31, SEQ ID NO:35, SEQ ID NO:39, SEQ ID NO:43, and SEQ ID NO:47;
(b) a protein comprising the HA1 region of an HA having an amino acid sequence selected from the group consisting of SEQ ID NO:27, SEQ ID NO:31, SEQ ID NO:35, SEQ ID NO:39, SEQ ID NO:43, and SEQ ID NO:47; and,
(c) a protein comprising an amino acid sequence selected from the group consisting of SEQ ID NO:27, SEQ ID NO:31, SEQ ID NO:35, SEQ ID NO:39, SEQ ID NO:43, and SEQ ID NO:47.

7-9. (canceled)

10. The immunogen of claim 1, wherein said glycan-shielded RBD-A region comprises at least one region selected from the group consisting of:
(a) amino acids 131-143 from SEQ ID NO:27 or SEQ ID NO:31;
(b) amino acids 170-182 from SEQ ID NO:27 or SEQ ID NO:31;
(c) amino acids 205-215 from SEQ ID NO:27 or SEQ ID NO:31;
(d) amino acids 257-262 from SEQ ID NO:27 or SEQ ID NO:31; and
(e) amino acids 131-146 from SEQ ID NO:27 or SEQ ID NO:31.

11-12. (canceled)

13. The immunogen of claim 1, wherein said nucleic acid construct comprises a nucleic acid sequence selected from the group consisting of SEQ ID NO:25, SEQ ID NO:29, SEQ ID NO:33, SEQ ID NO:37, SEQ ID NO:41, and SEQ ID NO:45.

14-18. (canceled)

19. A method to elicit a neutralizing antibody immune response against an influenza A subtype H1 virus in a subject comprising administering to said subject the immunogen of claim 1.

20-23. (canceled)

24. A method to protect a subject from influenza A subtype H1 infection comprising administering to said subject the immunogen of claim 1.

25. A protein comprising at least a portion of a hemagglutinin antigen having an amino acid sequence selected from the group consisting of SEQ ID NO:27, SEQ ID NO:31, SEQ ID NO:35, SEQ ID NO:39, SEQ ID NO:43, and SEQ ID NO:47.

26. The protein of claim 25, wherein said portion comprises at least one region selected from the group consisting of:
(a) amino acids 131-143 from SEQ ID NO:27 or SEQ ID NO:31;
(b) amino acids 170-182 from SEQ ID NO:27 or SEQ ID NO:31;
(c) amino acids 205-215 from SEQ ID NO:27 or SEQ ID NO:31;
(d) amino acids 257-262 from SEQ ID NO:27 or SEQ ID NO:31; and
(e) amino acids 131-146 from SEQ ID NO:27 or SEQ ID NO:31.

27. The protein of claim 25, wherein said protein comprises the receptor binding domain from a hemagglutinin antigen comprising SEQ ID NO:27, SEQ ID NO:31, SEQ ID NO:35, SEQ ID NO:39, SEQ ID NO:43, and SEQ ID NO:47.

28. The protein of claim 25, wherein said protein comprises the RBD-A region from a hemagglutinin antigen comprising SEQ ID NO:27 or SEQ ID NO:31.

29. The protein of claim 25, wherein said protein comprises SEQ ID NO:27, SEQ ID NO:31, SEQ ID NO:35, SEQ ID NO:39, SEQ ID NO:43, and SEQ ID NO:47.

30. (canceled)

31. A nucleic acid molecule encoding the protein of claim 25.

32. An immunogen comprising a nucleic acid construct comprising a nucleic acid molecule that encodes an immunogenic protein comprising at least one epitope of the receptor binding domain A (RBD-A) region of a pandemic influenza A subtype H1 hemagglutinin antigen, wherein said encoded RBD-A region is lacking any N-linked glycosylation site that is present in the RBD-A region of a non-pandemic influenza A subtype H1 hemagglutinin antigen, wherein said immunogenic protein elicits a neutralizing antibody immune response against a homologous pandemic influenza A subtype H1 virus strain and against a heterologous pandemic influenza A subtype H1 virus strain.

33. The immunogen of claim 32, wherein said pandemic influenza A subtype H1 hemagglutinin antigen is selected from the group consisting of a 1918 pandemic influenza A subtype H1 hemagglutinin antigen, a 1976 pandemic influenza A subtype H1 hemagglutinin antigen, and a 2009 pandemic influenza A subtype H1 hemagglutinin antigen.

34-35. (canceled)

36. The immunogen of claim 32, wherein said N-linked glycosylation site is selected from the group consisting of:
(a) an N-linked glycosylation site corresponding to amino acid position 142 of SEQ ID NO:3;
(b) an N-linked glycosylation site corresponding to amino acid position 144 of SEQ ID NO:3;
(c) an N-linked glycosylation site corresponding to amino acid position 172 of SEQ ID NO:3;
(d) an N-linked glycosylation site corresponding to amino acid position 177 of SEQ ID NO:3;
(e) an N-linked glycosylation site corresponding to amino acid position 179 of SEQ ID NO:3; and
(f) an N-linked glycosylation site corresponding to amino acid position 136 of SEQ ID NO:3.

37. The immunogen of claim 32, wherein the immunogenic protein is selected from the group consisting of:
(a) an immunogenic protein comprising at least one region selected from the group consisting of:
(1) amino acids 131-143 from SEQ ID NO:3 or SEQ ID NO:62;
(2) amino acids 170-182 from SEQ ID NO:3 or SEQ ID NO:62;
(3) amino acids 131-146 from SEQ ID NO:3 or SEQ ID NO:62; and
(4) amino acids 257-262 from SEQ ID NO:3 or SEQ ID NO:62; and,
(b) an immunogenic protein comprising:
(1) amino acids 131-143 from SEQ ID NO:3 or SEQ ID NO:62;
(2) amino acids 170-182 from SEQ ID NO:3 or SEQ ID NO:62;
(3) amino acids 205-215 from SEQ ID NO:3 or SEQ ID NO:62; and
(4) amino acids 257-262 from SEQ ID NO:3 or SEQ ID NO:62.

38. (canceled)

39. The immunogen of claim 32, wherein said immunogenic protein comprises at least one epitope from the RBD-A region of a hemagglutinin antigen comprising an amino acid sequence selected from the group consisting of SEQ ID NO:3, SEQ ID NO:7, SEQ ID NO:19, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, and SEQ ID NO:62.

40. (canceled)

41. The immunogen of claim 32 wherein said immunogenic protein comprises a hemagglutinin antigen comprising an amino acid sequence selected from the group consisting of SEQ ID NO:3, SEQ ID NO:7, SEQ ID NO:19, SEQ ID NO:49, and SEQ ID NO:62.

42-44. (canceled)

45. The immunogen of claim 32, wherein said nucleic acid construct comprises a nucleic acid sequence selected from the group consisting of SEQ ID NO:1, SEQ ID NO:5, SEQ ID NO:17, and SEQ ID NO:63.

46-50. (canceled)

51. A method to elicit a neutralizing antibody immune response against a pandemic influenza A subtype H1 virus comprising administering to a subject an immunogen comprising a nucleic acid molecule encoding a pandemic influenza A subtype H1 hemagglutinin antigen (HA), wherein said HA is heterologous to the virus against which an immune response is being elicited, and wherein said immunogen elicits said immune response.

52. The method of claim 51, wherein said hemagglutinin antigen is selected from the group consisting of influenza A/California/04/2009 HA and influenza A/South Carolina/1/1918 HA.

53. The method of claim 51, wherein said hemagglutinin antigen lacks any N-linked glycosylation site that is present in the receptor binding domain A (RBD-A) region of a hemagglutinin antigen from a non-pandemic influenza A virus.

54. The method of claim 53, wherein said N-linked glycosylation site is selected from the group consisting of:
(a) an N-linked glycosylation site corresponding to amino acid position 142 of SEQ ID NO:3;
(b) an N-linked glycosylation site corresponding to amino acid position 144 of SEQ ID NO:3;
(c) an N-linked glycosylation site corresponding to amino acid position 172 of SEQ ID NO:3;
(d) an N-linked glycosylation site corresponding to amino acid position 177 of SEQ ID NO:3; and,
(e) an N-linked glycosylation site corresponding to amino acid position 179 of SEQ ID NO:3; and
(f) an N-linked glycosylation site corresponding to amino acid position 136 of SEQ ID NO:3.

55. A method of claim 51, wherein said immunogen provides protection against a pandemic influenza A subtype H1 virus.

56. A method to elicit a neutralizing antibody immune response against an influenza A subtype H1 virus in a subject comprising administering to the subject the immunogen of claim 32.

57. A method to protect a subject against a pandemic influenza A subtype H1 virus comprising administering to said subject the immunogen of claim 32.

58. A method to reduce pandemic influenza A subtype H1 virus in an animal reservoir comprising administering to animals in said reservoir the immunogen of claim 32.

59. An immunogen comprising nucleic acid construct VRC 9328.

60-70. (canceled)

71. A method to detect the emergence of a non-pandemic influenza A subtype H1 virus from a pandemic population of influenza A subtype H1 virus, said method comprising:
(a) isolating a biological sample containing influenza A virus; and
(b) testing the hemagglutinin antigen of said virus for the presence of N-linked glycans at positions corresponding to amino acids 136, 142, 144, 172, 177 and 179 of SEQ ID NO:3;
wherein the presence of glycan at any of said positions indicates the emergence of a non-pandemic virus.
